(19) United States
(12) Patent Application Publication
(10) Pub. No.: US 2005/0026987 A1
(43) Pub. Date: Feb. 3, 2005

(54) CBI analogues of the duocarmycins and CC-1065

(75) Inventor: Dale L. Boger, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 10/846,027

(22) Filed: May 13, 2004

Related U.S. Application Data
(60) Provisional application No. 60/470,539, filed on May 13, 2003.

(51) Int. Cl. A61K 31/405; C 07D 4 3/02

(52) U.S. Cl. 514/410; 514/411; 548/420; 548/427

(57) ABSTRACT

An extensive series of CBI analogues of the duocarmycins and CC-1065 exploring substituent effects within the first indole DNA binding subunit is detailed. In general, substitution at the indole C5 position led to cytotoxic potency enhancements that can be ≧1000-fold providing simplified analogues containing a single DNA binding subunit that are more potent (IC₅₀=2-3 pM) than CBI-TMI, duocarmycin SA, or CC-1065. The increases in cytotoxicity correlate well with accompanying increases in the rate and efficiency of DNA alkylation. This effect is more pronounced with the CBI versus DSA or CPI based analogues. Moreover, this effect is largely insensitive to the electronic character of the C5 substituent but is sensitive to the size, rigid length, and shape (sp, sp², sp³ hybridization) of this substituent consistent with expectation that the impact is due simply to its presence.

### CROSS-REFERENCE TO RELATED APPLICATION

This is a non-provisional application of and claims priority from copending U.S. provisional application Ser. No. 60/470,539, filed May 13, 2003.

### GOVERNMENT RIGHTS

This invention was made, in part, with government support under a Grant from NIH, viz., Grant No. CA41986. The U.S. government may have certain rights in the invention.

### TECHNICAL FIELD

The invention relates to cytotoxic anti-cancer agents having DNA alkylating activity. More particularly, the invention relates to analogues of the duocarmycins and CC-1065 incorporating a CBI alkylation subunit (1,2,9,9a-tetrahydrocyclo-propa[c]benz[e]indol-4-one).

### BACKGROUND

CC-1065 (1), duocarmycin A (2) and duocarmycin SA (3) constitute the parent members of a class of potent antitumor antibiotics that derive their properties through a sequence-selective alkylation of duplex DNA, FIG. 1. Recent studies have established that the catalysis of the DNA alkylation reaction is derived from a DNA binding-induced conformational change in the agents which activate them for nucleophilic attack (Boger, D. L.; Garbaccio, R. M. Bioorg. Med. Chem. 1999, 5, 263-276; Boger, D. L.; Garbaccio, R. M. Acc. Chem. Res. 1999, 32, 1043-1052; Wolkenberg, S. W.; Boger, D. L. Chem. Rev. 2002, 102, 2477-2496; Boger, D. L.; et al. J. Am. Chem. Soc. 1997, 119, 4987-4998; Boger, D. L.; et al. J. Am. Chem. Soc. 1997, 119, 4977-4986; Ambroise, Y.; Boger, D. L. Bioorg. Med. Chem. Lett. 2002, 12, 303-306). This conformational change twists the amide linking the alkylation subunit and attached DNA binding domain which disrupts the cross-conjugated vinylogous amide stabilization of the alkylation subunit activating the cyclopropane for nucleophilic attack. This ground-state destabilization of the cyclopropane upon DNA binding is consistent with the proposal that the DNA alkylation sequence selectivity originates in the noncovalent binding selectivity of the agents (Boger, D. L.; Johnson, D. S. Angew. Chem., Int. Ed. Engl. 1996, 35, 1438-1474; For earlier reviews, see: Boger, D. L. Acc. Chem. Res. 1995, 28, 20-25; Boger, D. L.; Johnson, D. S. Proc. Natl. Acad. Sci., U.S.A. 1995, 92, 3642-3649; Boger, D. L. Chemtracts: Org. Chem. 1991, 4, 329-349; Synthesis: Boger, D. L.; et al. Chem. Rev. 1997, 97, 787-828).

Recent studies have indicated that the role of attached DNA binding domain goes beyond that of simply providing DNA binding affinity and selectivity, but that it contributes to and is largely responsible for the DNA alkylation catalysis (Boger, D. L.; Garbaccio, R. M. Bioorg. Med. Chem. 1999, 5, 263-276; Boger, D. L.; Garbaccio, R. M. Acc. Chem. Res. 1999, 32, 1043-1052; Wolkenberg, S. W.; Boger, D. L. Chem. Rev. 2002, 102, 2477-2496; Boger, D. L.; et al. J. Am. Chem. Soc. 1997, 119, 4987-4998; Boger, D. L.; et al. J. Am. Chem. Soc. 1997, 119, 4977-4986; Ambroise, Y.; Boger, D. L. Bioorg. Med. Chem. Lett. 2002, 12, 303-306; For an alternative source of catalysis, see: Ellis, D. A.; et al. J. Am. Chem. Soc. 2001, 123, 9299-9306; Boger, D. L.; et al. J. Am. Chem. Soc. 2000, 122, 6325-6326; Boger, D. L. Boyce, C. W. J. Org. Chem. 2000, 65, 4088-4100; For an alternative source of activation, see: Boger, D. L.; Garbaccio, R. M. J. Org. Chem. 1999, 64, 8350-8362; Boger, D. L.; et al. J. Org. Chem. 2001, 66, 6654-6661; Boger, D. L.; et al. Bioorg. Med. Chem. Lett. 2001, 11, 2021-2024). Minor groove bound substituents on both the first DNA binding subunit (Boger, D. L.; et al. J. Am. Chem. Soc. 1997, 119, 4987-4998; Boger, D. L.; et al. J. Am. Chem. Soc. 1997, 119, 4977-4986; Ambroise, Y.; Boger, D. L. Bioorg. Med. Chem. Lett. 2002, 12, 303-306; For an alternative source of catalysis, see: Ellis, D. A.; et al. J. Am. Chem. Soc. 2001, 123, 9299-9306; Boger, D. L.; et al. J. Am. Chem. Soc. 2000, 122, 6325-6326; Boger, D. L. Boyce, C. W. J. Org. Chem. 2000, 65, 4088-4100; For an alternative source of activation, see: Boger, D. L.; Garbaccio, R. M. J. Org. Chem. 1999, 64, 8350-8362; Boger, D. L.; et al., J. Org. Chem. 2001, 66, 6654-6661; Boger, D. L.; et al. Bioorg. Med. Chem. Lett. 2001, 11, 2021-2024; Boger, D. L.; et al. Bioorg. Med. Chem. Lett. 1996, 6, 2207-2212) and the alkylation subunit (Boger, D. L.; et al. J. Org. Chem. 1996, 61, 4894-4912; Boger, D. L.; et al. J. Org. Chem. 1996, 61, 1710-1729; Boger, D. L.; et al. J. Org. Chem. 2000, 65, 4101-4111; Boger, D. L.; et al. J. Org. Chem. 2001, 66, 2207-2216) have been shown to have a pronounced effect on the rate and efficiency of DNA alkylation and the resulting biological potency of the compounds. These effects proved to be independent of the electronic properties of the substituent and their inherent effects on reactivity, but could be attributed to their simple presence and the fact that they extend the rigid length of the agent. In doing so, they increase the extent of the DNA binding-induced conformational change, increase the degree of vinylogous amide disruption, and increase the rate of DNA alkylation. For example, the contribution of each of the three methoxy groups of 5,6,7-trimethoxyindole (TMI) was established using the DNA alkylation subunits DSA (Boger, D. L.; et al. J. Am. Chem. Soc. 1997, 119, 4977-4986) and CPI (Boger, D. L.; et al. J. Org. Chem. 2000, 65, 4101-4111). These studies demonstrated the predominant importance of the C5 methoxy substituent, which alone provided a fully active agent, with little or no contribution derived from the C6 and C7 methoxy groups. Moreover, the cytotoxic potency of these agents nicely correlated with their DNA alkylation rate and efficiency. The conclusion being that the agents bearing a C5 methoxy substituent were more effective and that this was due to the extended rigid length provided by the minor groove bound substituent. Subsequently, this was found to be consistent with the high resolution NMR structures of (+)-duocarmycin SA (Eis, P. S.; et al. J. Mol. Biol. 1997, 272, 237-252) and its derivative DSI (DSA-indole) (Schnell, J. R.; et al. J. Am. Chem. Soc. 1999, 121, 5645-5652), lacking the three methoxy groups, bound to DNA which confirmed that the presence of C5 methoxy group increased the twist in the DNA bound agent (Smith, J. A.; et al. J. Mol. Biol. 2000, 300, 1195-1204). Moreover, the C5 methoxy group of 3 is found deeply embedded in the minor groove with methyl group extending into, not away from, the minor groove floor, potentially benefiting from hydrophobic contacts (FIG. 2).

Despite these studies and reports of limited series of agents (Warpehoski, M. A.; et al. J. Med. Chem. 1988, 31, 590-603; Atwell, G. J.; et al. J. Med. Chem. 1999, 42, 3400-3411; Boger, D. L.; et al. Bioorg. Med. Chem. 1995, 3, 761-775), no systematic examination of the DNA binding subunit C5 substituent has been disclosed. Following observations made in an earlier study (Boger, D. L.; et al. Bioorg. Med. Chem. Lett. 2001, 11, 2021-2024), and utilizing the CBI alkylation subunit (1,2,9,9a-tetrahydrocyclo-propa[c]benz[e]indol-4-one) (Boger, D. L.; et al. J. Org. Chem. 2001, 66, 6654-6661; Boger, D. L.; et al. Bioorg. Med. Chem. Lett. 2001, 11, 2021-2024; Boger, D. L.; et al. Bioorg. Med. Chem. Lett. 1996, 6, 2207-2212; Boger, D. L.; et al. J. Org. Chem. 1996, 61, 4894-4912; Boger, D. L.; et al. J. Org. Chem. 1996, 61, 1710-1729; Boger, D. L.; et al. J. Org. Chem. 2000, 65, 4101-4111; Boger, D. L.; et al. J. Org. Chem. 2001, 66, 2207-2216; Eis, P. S.; et al. J. Mol. Biol. 1997, 272, 237-252; Schnell, J. R.; et al. J. Am. Chem. Soc. 1999, 121, 5645-5652; Smith, J. A.; et al. J. Mol. Biol. 2000, 300, 1195-1204; Warpehoski, M. A.; et al. J. Med. Chem. 1988, 31, 590-603; Atwell, G. J.; et al. J. Med. Chem. 1999, 42, 3400-3411; Boger, D. L.; et al. Bioorg. Med. Chem. 1995, 3, 761-775; Boger, D. L.; et al. J. Am. Chem. Soc. 1989, 111, 6461-6463; Boger, D. L.; et al. J. Org. Chem. 1990, 55, 5823-5832; Boger, D. L.; Ishizaki, T. Tetrahedron Lett. 1990, 31, 793-796; Boger, D. L.; et al. Bioorg. Med. Chem. Lett. 1991, 1, 115-120; Boger, D. L.; et al. J. Am. Chem. Soc. 1991, 113, 2779-2780), herein are described an extensive library of nearly 80 derivatives designed to establish the impact of substituents placed on the first DNA binding subunit. The CBI subunit was chosen because it represents the synthetically most accessible simplified alkylation subunit in the series (Boger, D. L.; et al. J. Org. Chem. 1992, 57, 2873-2876; Boger D. L.; McKie, J. A. J. Org. Chem. 1995, 60, 1271-1275; Boger D. L.; et al. Synlett 1997, 515-517; Boger D. L.; et al. Tetrahedron Lett. 1998, 39, 2227-2230), exhibits a potency and efficacy that surpasses that found in 1 and 2 while approaching that of 3 (Boger, D. L.; et al. J. Am. Chem. Soc. 1989, 111, 6461-6463; Boger, D. L.; et al. J. Org. Chem. 1990, 55, 5823-5832; Boger, D. L.; Ishizaki, T. Tetrahedron Lett. 1990, 31, 793-796; Boger, D. L.; et al. Bioorg. Med. Chem. Lett. 1991, 1, 115-120), exhibits stability and reaction regioselectivity characteristics that are near optimal (Boger, D. L.; et al. J. Am. Chem. Soc. 1991, 113, 2779-2780), and constitutes a parent ring system that has been extensively employed, extended, or modified. For an alternative source of catalysis, see: Ellis, D. A.; et al. J. Am. Chem. Soc. 2001, 123, 9299-9306; Boger, D. L.; et al. J. Am. Chem. Soc. 2000, 122, 6325-6326; Boger, D. L. Boyce, C. W. J. Org. Chem. 2000, 65, 4088-4100. For an alternative source of activation, see: Boger, D. L.; Garbaccio, R. M. J. Org. Chem. 1999, 64, 8350-8362; Boger, D. L.; et al. J. Org. Chem. 2001, 66, 6654-6661; Boger, D. L.; et al. J. Org. Chem. 1996, 61, 4894-4912; Boger, D. L.; et al. J. Org. Chem. 1996, 61, 1710-1729; Boger, D. L.; et al. J. Org. Chem. 2001, 66, 2207-2216; C3-substituted CBI analogues: Boger, D. L.; et al. J. Org. Chem. 2001, 66, 5163-5173; Iso-CBI: Boger, D. L.; et al. J. Org. Chem. 1997, 62, 8875-8891; C₂BI: Boger, D. L.; et al. J. Am. Chem. Soc. 1992, 114, 9318-9327; Boger, D. L.; et al. Bioorg. Med. Chem. 1993, 1, 27-38; CBQ: Boger, D. L.; Mesini, P. J. Am. Chem. Soc. 1995, 117, 11647-11655; Boger, D. L.; Mesini, P. J. Am. Chem. Soc. 1994, 116, 11335-11348; CNA: Boger, D. L.; Turnbull P. J. Org. Chem. 1997, 62, 5849-5863; CBIn: Boger, D. L.; Turnbull, P. J. Org. Chem. 1998, 63, 8004-8011; Boger, D. L.; et al. Synthesis 1999, 1505-1509; Boger, D. L.; et al. J. Am. Chem. Soc. 1998, 120, 11554-11557; Boger, D. L.; et al. J. Org. Chem. 1999, 64, 5241-5244; Boger, D. L.; Garbaccio, R. M. J. Org. Chem. 1999, 64, 5666-5669; Boger, D. L.; et al. Bioorg. Med. Chem. Lett. 1997, 7, 233-238; Chang, A. Y.; Dervan, P. B. J. Am. Chem. Soc. 2000, 122, 4856-4864; Kumar, R.; Lown, J. W. Org. Lett. 2002, 4, 1851-1854; Reddy, B. S. P.; et al. Curr. Med. Chem. 2001, 8, 475-508; Denny, W. A. Curr. Med. Chem. 2001, 8, 533-544; Jia, G. F.; Lown, J. W. Bioorg. Med. Chem. 2000, 8, 1607-1617; Jia, G. F.; et al. Synlett 2000, 603; Wang, Y. Q.; et al. J. Med. Chem. 2000, 43, 1541-1549; Jia, G. F.; et al. Heterocyclic Commun. 1999, 5, 497-502; Hay, M. P.; et al. Brit. J. Cancer. 1999, Suppli. 2, P65; Hay, M. P.; et al. Bioorg. Med. Chem. Lett. 1999, 9, 2237-2242; Gieseg, M. A.; et al. Anti-Cancer Drug Design 1999, 14, 77-84; Jia, G. F.; et al. Heterocyclic Commun. 1998, 4, 557-560; Jia, G. F.; et al. Chem. Commun. 1999, 119-120; Atwell, G. J.; et al. J. Org. Chem. 1998, 63, 9414-9420; Atwell, G. J.; et al. Bioorg. Med. Chem. Lett. 1997, 7, 1493-1496; Chari, R. V. J.; et al. Cancer Res. 1995, 55, 4079-4084; Wang, Y.; et al. U.S. patent 1998, 5843937).

### SUMMARY

One aspect of the invention is directed to a compound having a formula selected from the group consisting of the following structures:

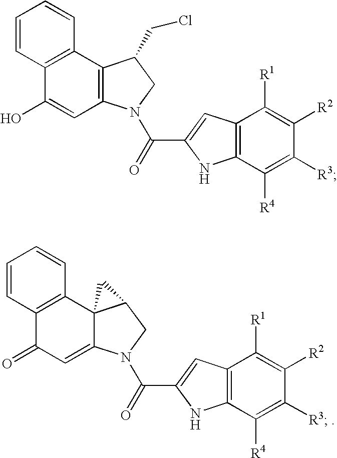

In the above structures, R¹ is —H or forms a five membered ring with R²; R² is selected from the group consisting of —COOR^{a}, —CONR^{b}R^{c}, S(O)NR^{d}, —OCF₃, —NO₂, —CHO, —CH═CH₂, —C(CH₃)═CH₂, and —CH₂OCH₃ or forms a five membered ring with either R¹ or R³; R³ is selected from the group consisting of —H, —OCF₃, —CHO, —NO₂, —CH═CH₂, —C(CH₃)═CH₂, and —CH₂OCH₃, or forms a five membered ring with R²; and R⁴ is selected from the group consisting of —H, —OCF₃, —CHO, —NO_{2,} —CH═CH₂, —C(CH₃)═CH₂, and —CH₂OCH₃; R^{a}, R^{b}, R^{c, and R}^{d} are independently —C₍₁₋₆₎ alkyl radicals; n is 1 or 2; and the five membered ring being of a type selected from the group consisting of five membered rings having 5 ring carbons having a ketone substitution, five membered rings having 4 ring carbons and 1 ring oxygen, and five membered rings having 3 ring carbons and 2 nonadjacent ring oxygens, each of the five membered rings having 1 ring unsaturation. However, the following provisos apply:
1.) at least one of R¹, R², R³, and R⁴ is not —H; and
2.) if R² forms a five membered ring, then R² forms a five membered ring with only one of R¹ or R³.

Another aspect of the invention is directed to a compound having a formula selected from the group represented by the following structures: 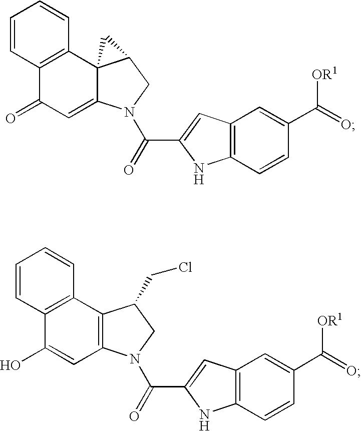
In the above structures, R¹ is a —(C₁-C₆)alkyl radical. Preferred embodiments include compounds represented by the following structures:

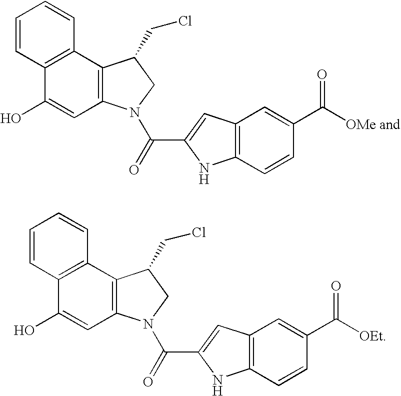

Another aspect of the invention is directed to a compound having a formula selected from the group represented by the following structures: 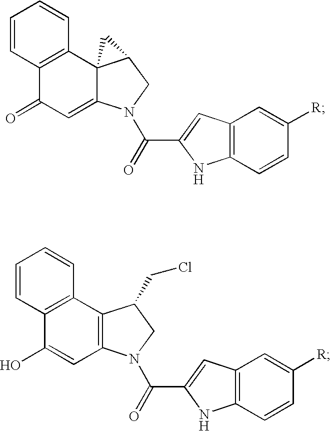
In the above structures, R is —CONR¹R²; and R¹ and R² are each independently —(C₁-C₆)alkyl radicals. Preferred embodiments include compounds represented by the following structures:

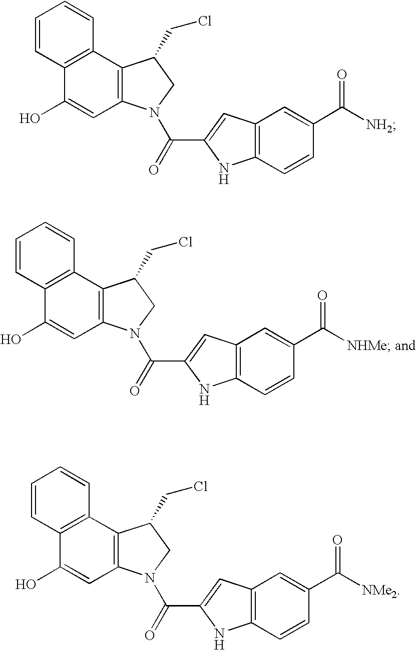

Another aspect of the invention is directed to a compound having a formula selected from the group represented by the following structures: 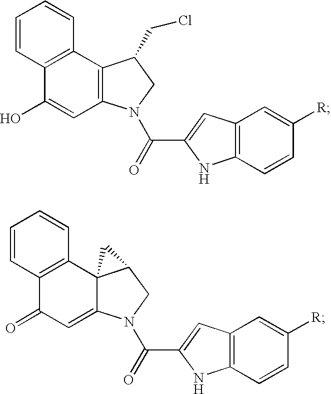
In the above structures, R is —S(O)ₙR¹; n is 1 or 2; and R¹ is a —(C₁-C₆) radical. Preferred embodiments include compounds represented by the following structures:

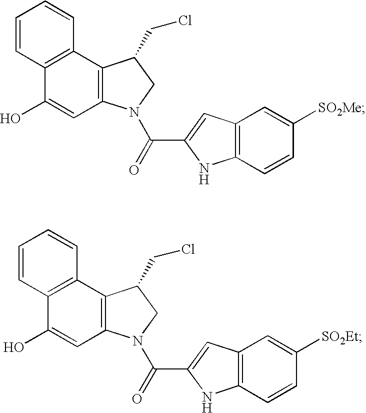

Another aspect of the invention is directed to a compound having a formula selected from the group represented by the following structures: 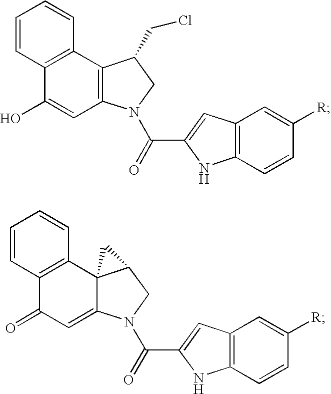
In the above structures, R is —SR¹; and R¹ is a —(C₁-C₆)alkyl radical. Preferred embodiments include compounds represented by the following structures:

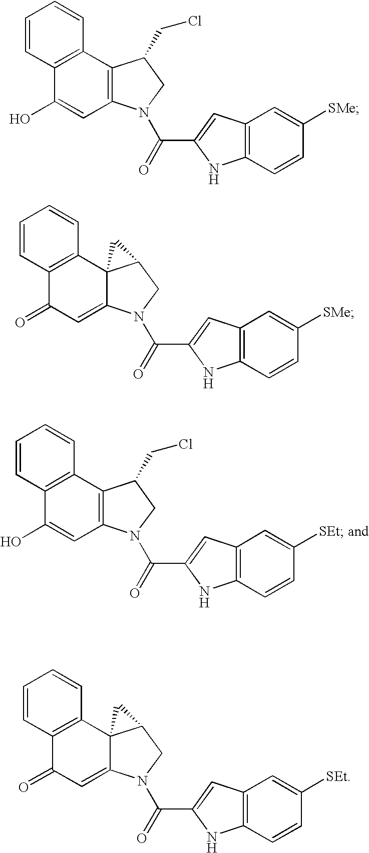

Another aspect of the invention is directed to a compound having a formula selected from the group represented by the following structures: 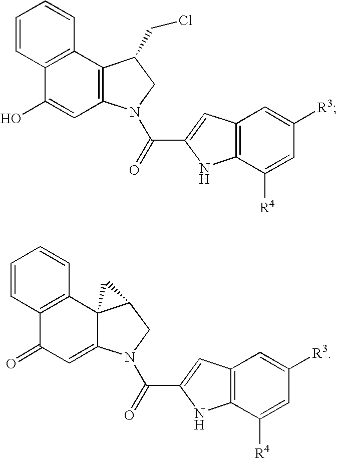
In the above structures, R³ and R⁴ are each independently selected from a group consisting of —OCF₃ and —H; with the provisos that, at least one of R³ and R⁴ is —OCF₃, and at least one of R³ and R⁴ is —H. Preferred embodiments include compounds represented by the following structures:

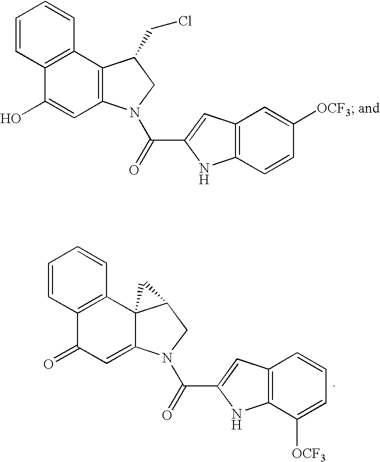

Additional preferred species of the invention are directed to the following compounds:

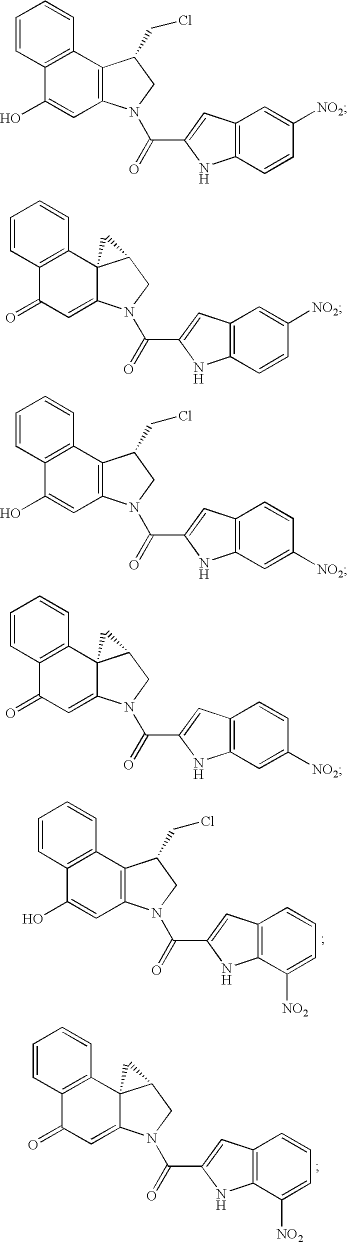

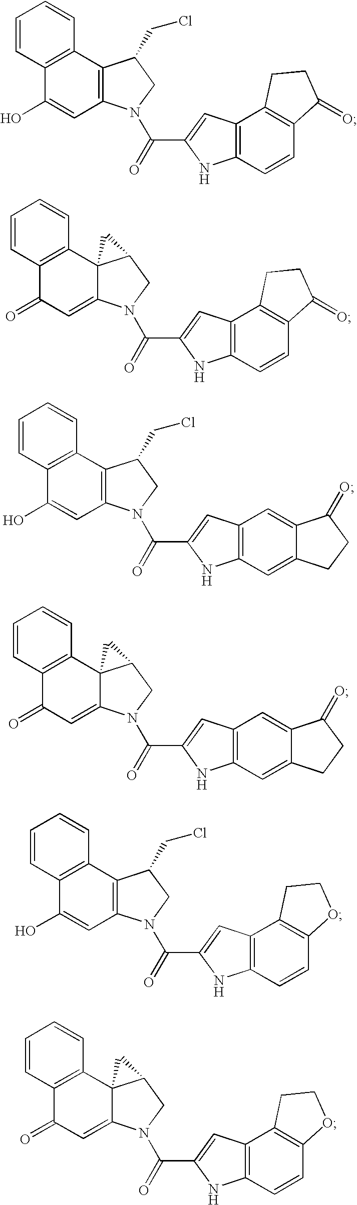

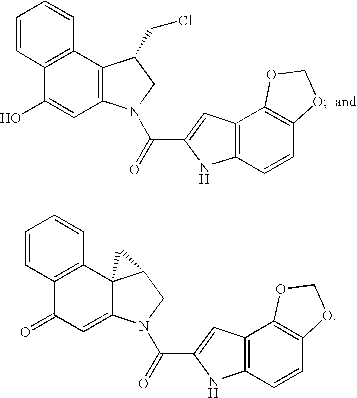

Another aspect of the invention is directed to a compound having a formula selected from the group represented by the following structures: 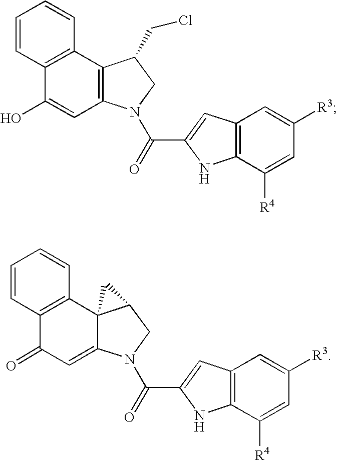
In the above structures, R³ and R⁴ are independently selected from the group consisting of —CHO and —H, with the provisos that at least one of R³ and R⁴ is —CHO, and at least one of R³ and R⁴ is —H. Preferred embodiments include compounds represented by the following structures:

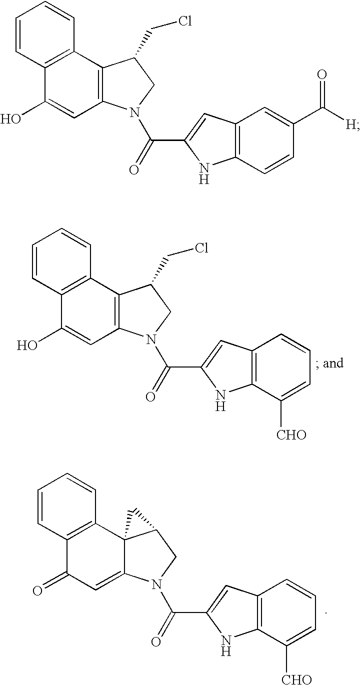

Additional preferred species of the invention are directed to the following compounds:

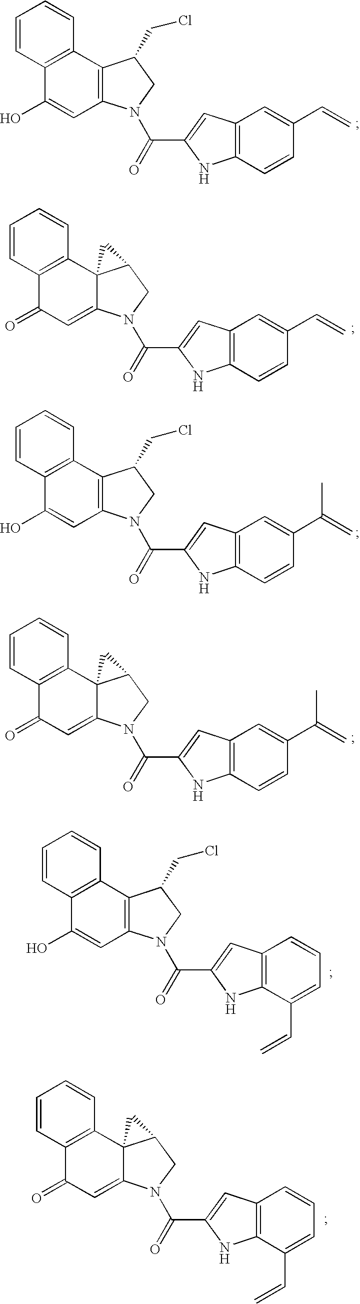

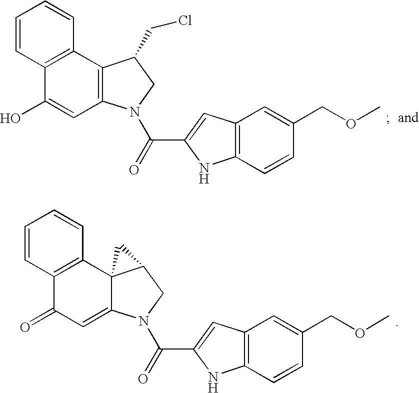

### BRIEF DESCRIPTION OF DRAWINGS

FIG. 1 shows the structures of CC-1065 (1), duocarmycin A (2), and duocarmycin SA (3).

FIG. 2 shows the front and groove view of the ¹H NMR derived solution structure of (+)-duocarmycin SA bound to a high affinity alkylation site within d(GACTAATTGAC)-d(GTCAATTAGTC) highlighting the minor groove embedded indole C5 methoxy group (Eis, P. S.; et al. J. Mol. Biol. 1997, 272, 237-252).

FIG. 3 is a scheme that shows the preparation of the compounds 6B-78B.

FIG. 4 is a table comparing the IC₅₀'s of the natural compounds to some analogs.

FIG. 5 is a table comparing the various compounds used in a systematic approach to finding the effects of the individual methoxy groups on the indole.

FIG. 6 is a table of compounds with a wider variety of C5 indole substituents.

FIG. 7 is a table showing a series of amine and amide derivatives of 5-aminoindole.

FIG. 8 is a table showing a series of three nitro-substituted indole derivatives and their relative potencies.

FIG. 9 is a table with carbonyl-containing substituents on the indole ring.

FIG. 10 is a drawing showing the location of the antibiotic in the minor groove after alkylation.

FIG. 11 is a table with two sulfone substituted indole derivatives.

FIG. 12 is a table comparing the potencies of the tricyclic indole derivatives.

FIG. 13 is a table showing the potency of the analogs which have a linear or angularly fused benzene ring on the indole.

FIG. 14 is a table with the relative alkylation efficiency and the relative rate of alkylation of selected derivatives.

FIG. 15 is a gel that shows the w794 DNA alkylation after 24 h and at 23° C. with four of the analogs at the 3′-ACTGATTAA-5′.

FIG. 16 is a synthetic scheme for the synthesis of indole carboxylic acids 104, 105 and 108.

FIG. 17 is a scheme for the synthesis of indole carboxylic acids 112, 113, and 114.

FIG. 18 is a scheme for the 5- and 7-trifluoromethoxyindole-2-carboxylic acids 118, and 119, respectively.

FIG. 19 is a scheme for the synthesis of thio and sulfonyl-substituted indole 2-carboxylic acids 125a, 125b, 126a, 126b and 127.

FIG. 20 is a scheme for the synthesis of methyl methoxy-substituted indole 2-carboxylic acids 132 and 133.

FIG. 21 is a scheme for the synthesis of 5-azidoindole-2-carboxylic acid 136.

FIG. 22 is a scheme for the synthesis of 5-cyanoindole-2-carboxylic acid 140.

FIG. 23 is a scheme for the synthesis of 7-cyanoindole-2-carboxylic acid 144.

FIG. 24 is a scheme for the synthesis of 2-carboxylic acid indoles 147, 150 and 151.

FIG. 25 is a scheme for the synthesis of 5- and 7-isopropenylindole-2-carboxylic acids 153 and 155.

FIG. 26 is a scheme for the synthesis of 5-Ethynylindole-2-carboxylic acid 157.

FIG. 27 is a scheme for the synthesis of 5-(1-Propynyl)-indole-2-carboxylic acid 159.

FIG. 28 is a scheme for the synthesis of 4-Phenylindole-2-carboxylic acid 163.

FIG. 29 is a scheme for the synthesis of 5-Dimethylaminoindole-2-carboxylic acid 165.

FIG. 30 is a scheme for the synthesis of 5-acetylaminoindole-2-carboxylic acid 172, 5-propionylaminoindole-2-carboxylic acid 173, and 5-butyrylaminoindole-2-carboxylic acid 174.

FIG. 31 is a scheme for the synthesis of 5-(N-acetyl-N-methylamino)indole-2-carboxylic acid 177 and 5-(N-acetyl-N-ethylamino)indole-2-carboxylic acid 179.

FIG. 32 is a scheme for the synthesis of 5-formylindole-2-carboxylic acid 181 and 7-formylindole-2-carboxylic acid 180.

FIG. 33 is a scheme for the synthesis of 5-acetylindole-2-carboxylic acid 183, 7-acetylindole-2-carboxylic acid 184, 5-propionylindole-2-carboxylic acid 187, and 5-butyrylindole-2-carboxylic acid 188.

FIG. 34 is a scheme for the synthesis of 7-methoxycarbonylindole-2-carboxylic acid 190 and 5-methoxycarbonylindole-2-carboxylic acid 192. The syntheses of 5-ethoxycarbonylindole-2-carboxylic acid 193, 5-methylcarbamoylindole-2-carboxylic acid 194, and 5-dimethylcarbamoylindole-2-carboxylic acid 195.

FIG. 35 is a scheme for the synthesis of 5-carbamoylindole-2-carboxylic acid 196.

FIG. 36 is a scheme for the synthesis of 1,2-Dihydropyrrolo[3,2-e]benzofuran-7-carboxylic acid (199).

FIG. 37 is a scheme for the synthesis of pyrrolo[3,2-e]benzofuran-7-carboxylic acid 202.

FIG. 38 is a scheme for the synthesis of 6-oxo-cyclopenten[e]indole-2-carboxylic acid 207.

FIG. 39 is a scheme for the synthesis of 6-oxo-cyclopenten[f]indole-2-carboxylic acid 212.

FIG. 40 is a scheme for the synthesis of [1,3]dioxolo[e]indole-2-carboxylic acid 215.

FIG. 41 is a scheme for the synthesis of [1,3]Dioxolo[f]indole-2-carboxylic acid 218.

FIG. 42 is a scheme for the synthesis of cyclopenten[e]indole-2-carboxylic acid 223.

FIG. 43 is a scheme for the synthesis of benz[e]indole-2-carboxylic acid 226.

FIG. 44 is a scheme for the synthesis of benz[f]indole-2-carboxylic acid 232.

FIG. 45 is a scheme showing the procedure for attaching the alkylation unit to the indol-2-carboxylic acids and for completing the spirocyclization to give the completed CBI alkylation subunit.

### DETAILED DESCRIPTION

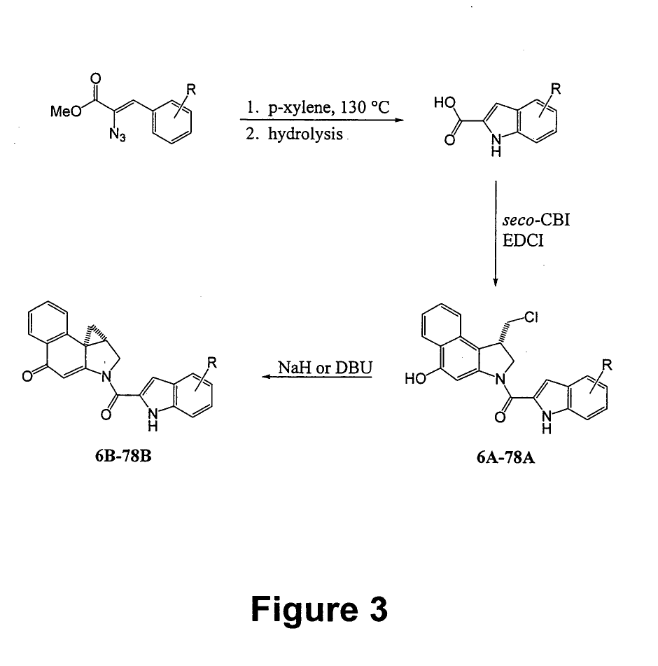

The compounds were prepared as shown in FIG. 3. The non-commercially available indole-2-carboxylic acids were obtained from the corresponding azido cinnamates derived from condensation of substituted benzaldehydes with methyl α-azidoacetate. The azidocinnamates were subjected to the Hemetsberger reaction (Hemetsberger, H.; et al. Montash. Chem. 1969, 100, 1599-1603) followed by saponification of the esters to provide the substituted indole-2-carboxylic acids. Acid-catalyzed deprotection of seco-N-BOC-CBI (natural or unnatural enantiomer) followed by coupling of the resulting hydrochloride salt with the indole-2-carboxylic acids (3 equiv EDCl, DMF, 25° C., 14 h) in the absence of added base provided the seco agents (Boger, D. L.; et al. J. Am. Chem. Soc. 1989, 111, 6461-6463; Boger, D. L.; et al. J. Org. Chem. 1990, 55, 5823-5832; Boger, D. L.; Ishizaki, T. Tetrahedron Lett. 1990, 31, 793-796; Boger, D. L.; et al. Bioorg. Med. Chem. Lett. 1991, 1, 115-120). Spirocyclization was effected by treatment with DBU (Boger, D. L.; et al. J. Am. Chem. Soc. 1998, 120, 11554-11557; Boger, D. L.; et al. J. Org. Chem. 1999, 64, 5241-5244) or NaH (Boger, D. L.; et al. J. Am. Chem. Soc. 1989, 111, 6461-6463; Boger, D. L.; et al. J. Org. Chem. 1990, 55, 5823-5832; Boger, D. L.; Ishizaki, T. Tetrahedron Lett. 1990, 31, 793-796; Boger, D. L.; et al. Bioorg. Med. Chem. Lett. 1991, 1, 115-120) providing 6B-78B. In the case of 77-78, both enantiomers of the compounds were examined, whereas in the case of 6-76 only the more potent natural enantiomers were examined.

Results:

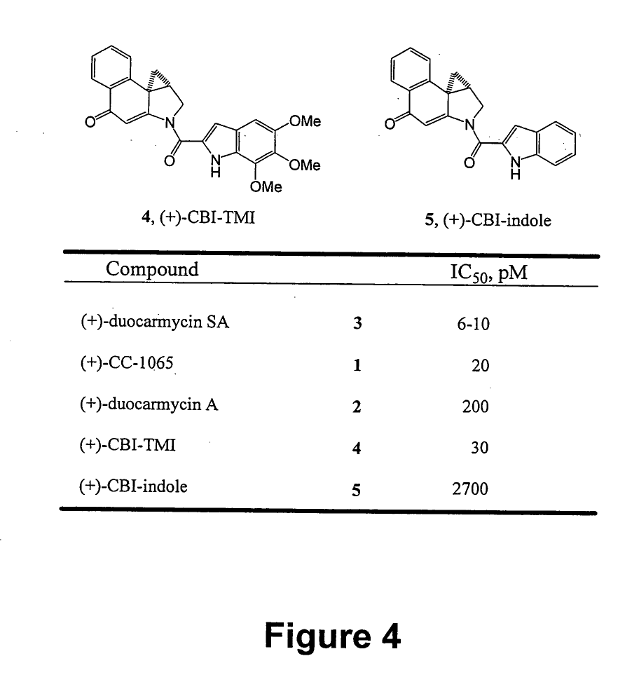

The comparison compounds for examining the effects of the indole substituents of the CBI analogues of duocarmycin SA (3) are CBI-TMI (4) (Boger D. L.; Yun, W. J. Am. Chem. Soc. 1994, 116, 7996-8006) and CBI-indole (5, FIG. 4). The former contains the 5,6,7-trimethoxy substituents of duocarmycin SA (3) while the latter incorporates the parent unsubstituted indole. (+)-CBI-TMI (4) was found to be nearly 100-fold more potent than (+)-CBI-indole (5) with the 5,6,7-trimethoxyindole substitution increasing the L1210 cytotoxic potency 90 times. This effect of the 5,6,7-trimethoxy substitution is analogous, but more pronounced, than the 6-10 fold effect observed with duocarmycin SA (Boger, D. L.; et al. J. Am. Chem. Soc. 1997, 119, 4977-4986) and CPI-TMI (Boger, D. L.; et al. J. Org. Chem. 2000, 65, 4101-4111).

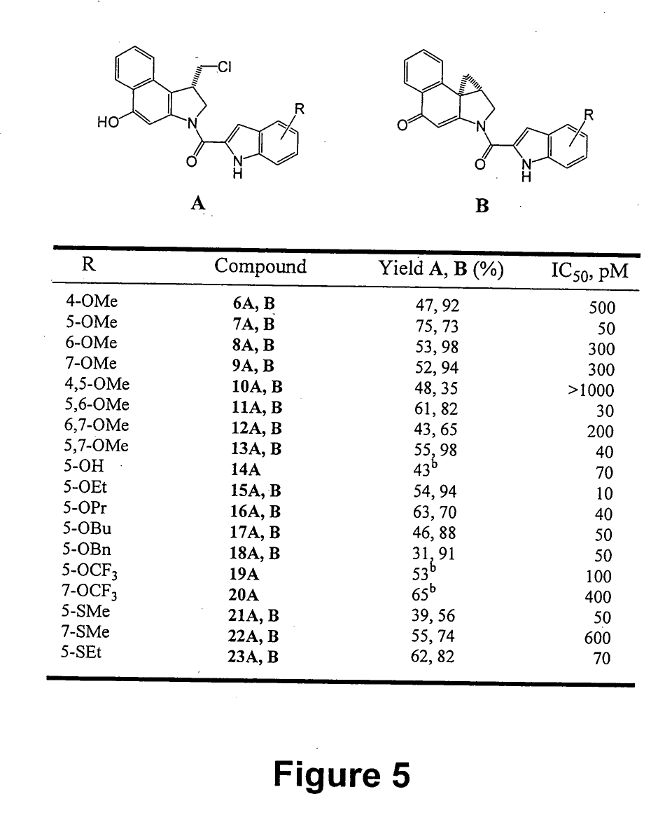

The comparisons in FIG. 5 detail the systematic approach taken to establish the effects of the individual methoxy groups found on the indole. Although the CBI-based agents proved to be more sensitive to the removal of the TMI subunit methoxy groups than the DSA- or CPI-based agents, analogous trends were observed. When the cytotoxic potency of CBI-TMI (4, IC₅₀=30 pM) (Warpehoski, M. A.; et al. Chem. Res. Toxicol. 1988, 1, 315-333; Boger D. L.; Yun, W. J. Am. Chem. Soc. 1994, 116, 7996-8006) was compared with the methoxy series 6 (C4 OMe), 7 (C5 OMe), 8 (C6 OMe), 9 (C7 OMe), decreases of 17×, 1.6×, 10×, and 10×, respectively, were observed. Thus, maintenance of the C5 methoxy group with removal of the C6 and C7 methoxy groups with analogue 7 maintained the cytotoxic potency, whereas its removal in 8 and 9 led to a ≧10× reduction in activity. Similar, but less pronounced, reductions in potency with the removal of the TMI methoxy substituents were observed with duocarmycin SA (6.5-10×) and CPI (7×). In the case of duocarmycin SA, the C7 methoxy substituent did not provide a contribution to cytotoxic potency and that of the C6 methoxy group was modest, whereas with CBI (7 vs. 8 and 9) both the C7 and C6 methoxy group were found to improve potency significantly, but not nearly of the magnitude observed with the C5 methoxy group. Most surprising of the effects was the relatively potent behavior of 6. Although it was 16-17 fold less active than CBI-TMI, it was 4-fold more active than CBI-indole despite the methoxy substitution at C4. A similar effect is seen with the dimethoxy derivatives 11 (C5,6 OMe), 12 (C6,7 OMe), and 13 (C5,7 OMe) where the potency of 11 (C5,6 OMe) and 13 (C5,7 OMe) were not distinguishable from that of 7 (C5 OMe) being only 1.0-1.3 fold from that of CBI-TMI, whereas 12 (C6,7 OMe) exhibited a more significant 6.6-fold reduction being essentially equivalent to the C6 or C7 monomethoxy derivatives 8 and 9. Clearly, the C5 position is the most important site potentiating the cytotoxic activity (C5 OMe>C6 OMe>C7 OMe>C4 OMe>H), and additional methoxy substitutions act in a more modest, but predictably additive manner. Interestingly, the C4,5 dimethoxy derivative 10 was much less active.

The effect of additional ether and thioether substituted indoles is also summarized in FIG. 5. Alterations in the C5 methoxy group providing longer, flexible, and more hydrophobic alkyl ethers (15-18) had little effect on the cytotoxic potency although a significant and optimal additional 5-fold increase was observed with the C5 ethoxy derivative (15, IC₅₀=10 pM), and a reduction in potency was observed with the corresponding C5 free phenol 14 versus C5 methyl ether. Notably, the trifluoromethyl ether series with substitutions at the C5 and C7 positions revealed a possible deleterious effect derived from fluorine substitution on the substituents that lie embedded in the minor groove. A substantial decrease of 2-fold in cytotoxic activity was seen with the C5 OCF₃ group relative to 7 (C5 OMe), whereas the C7 OCF₃ substitution proved essentially equivalent with 9 (C7 OMe). A thioether series provided similar observations. The derivative bearing a C5 thiomethyl group had the same cytotoxic activity as the corresponding C5 OCH₃ derivative (IC₅₀=50 pM). Likewise, the C7 thiomethyl group was an order of magnitude less potent and comparable in activity to 9 (C7 OMe). Interestingly, the C5 thioethyl derivative did not exhibt the increased potency found with 15.

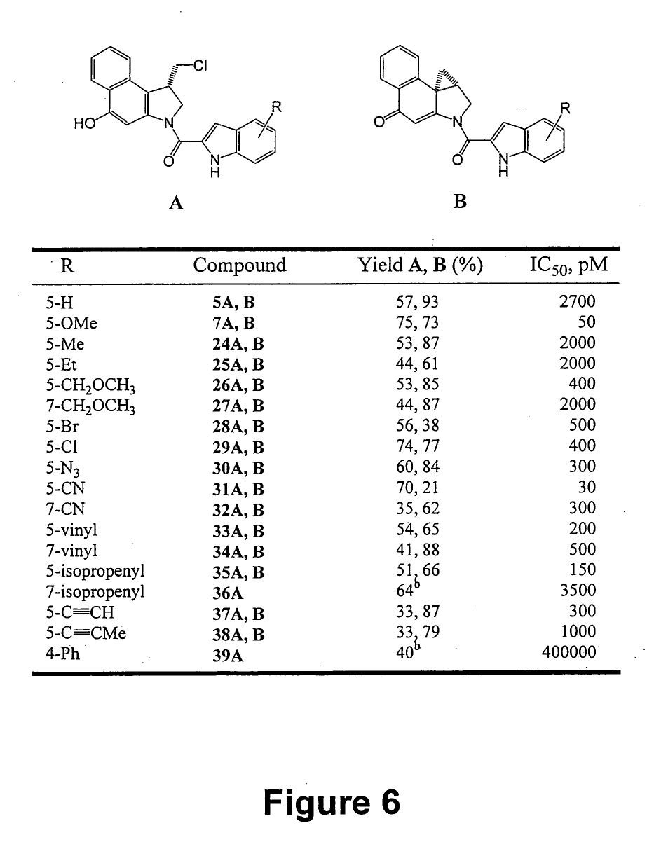

As a consequence of these observations, a wide range of C5 indole substituents were examined. Many of these are summarized in FIG. 6 and those that provided the most potent derivatives or that were examined as a comparison series are summarized in subsequent tables. Although no single generalization is easily discerned from the comparisons summarized in FIG. 6, some important trends emerge that are discussed in more detail with the subsequent series. Addition of a single heavy atom substituent typically resulted in a modest 1.3-40× increase in potency which diminishes as the size of the substituent increased (potency: OH═NH₂>Cl>Br>Me), unsaturated C5 substituents proved more potent than the corresponding saturated counterparts (potency: C≡CH═CH═CH₂>Et) consistent with restrictions on the optimal size of the C5 substiuent, extension of the rigid length of the unsaturated C5 substiuents smoothly follows the trend 0<1<2>3 heavy atoms, and branching at the site of C5 attachment with a small hydrophobic group may increase potency (MeC═CH₂>HC═CH₂). In each case, a C5 substituent was more potent than the corresponding C7 substituent, and the impact of the C5 substituent on potency appears to be related simply to its presence and shape characteristics while being relatively independent of its electronic properties (e.g., OMe=CN). Most noteworthy in this series is the potency of the simple nitrile 31 (IC₅₀=30 pM) which is equivalent with CBI-TMI and slightly more potent than 7 (C5 OMe, IC₅₀=50 pM).

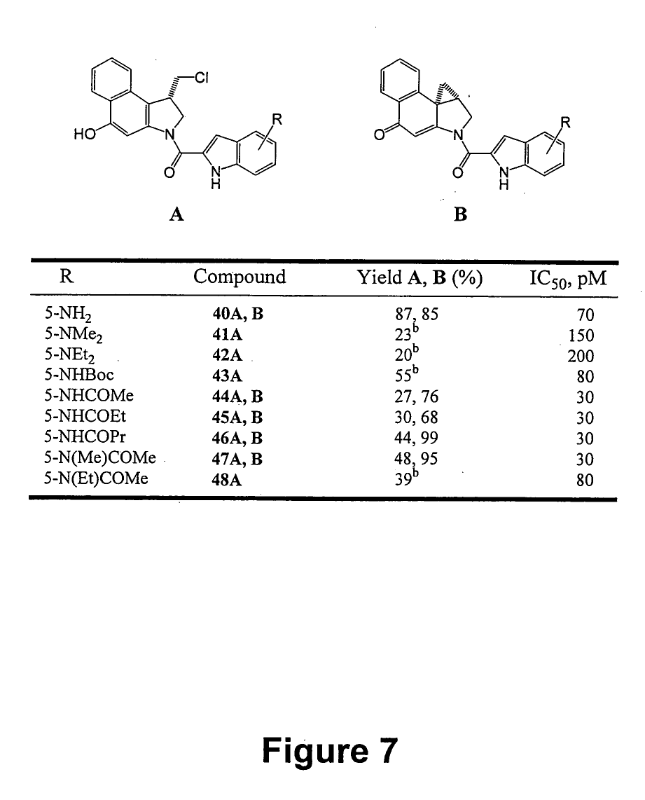

An important series examined early in the studies was the amine and amide derivatives of 5-aminoindole, FIG. 7. Not only does this represent the linking functionality and substitution found in the potent derivatives containing more than one DNA binding subunit (e.g., CBI indole₂ and 1), but Lown has reported significant effects of such amides within a select set of (+)-CPI-N-methylpyrrole agents constituting hybrid stuructures of CPI linked with the DNA binding subunits of distamycin (Wang, Y.; et al. Anti-Cancer Drug Design 1996, 11, 15-34). Notably the amide derivatives 44-47 proved to be potent cytotoxic agents (IC₅₀=30 pM) being slightly more potent than 7 (C5 OMe, IC₅₀=50 pM) and equivalent with (+)-CBI-TMI (IC₅₀=30 pM) even though they contain a single C5 substituent. This activity was invariant within the range of the amides examined (44=45=46), and was unaffected by N-methylation (44=47) indicating H-bonding plays no role in their activity. Notably, this series did not exhibit the trends detailed by Lown for the CPI-pyrrole conjugates (NHCOPr>NHCOEt>NHCOMe). Most likely, this may be attributed to the intrinsically poorer properties of the CPI-pyrrole conjugates (Boger, D. L.; et al. J. Org. Chem. 2001, 66, 6654-6661; Wang, Y.; et al. Anti-Cancer Drug Design 1996, 11, 15-34) reported by Lown and the greater influence such amide substituents may have.

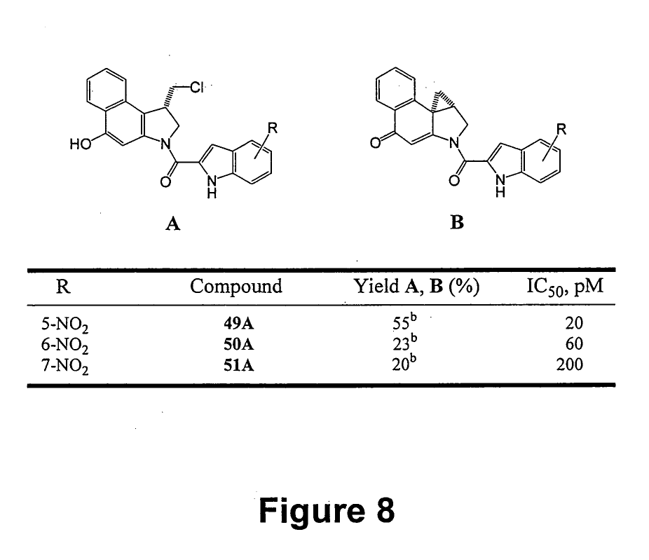

An interesting and perhaps unprecedented series that was examined exploring the 5, 6, and 7-nitroindoles is summarized in FIG. 8. Consistent with the trends exhibited with the methoxy substitution pattern (C5>C6>C7; FIG. 5) (Boger, D. L.; et al. J. Am. Chem. Soc. 1997, 119, 4977-4986; Boger, D. L.; et al. J. Org. Chem. 2000, 65, 4101-4111), the smooth trend of 49>50>51 was observed. The distinction being that each was more potent than the corresponding methoxy derivative with 49 (C5 NO₂, IC₅₀=20 pM) not only surpassing the potency of 7 (C5 OMe, IC₅₀=50 pM) but also surpassing the potency of even (+)-CBI-TMI (4, IC₅₀=30 pM). Nonetheless, the relatively small distinction between the strongly electron-withdrawing NO₂ versus electron-donating OMe substituent (C5=2.5×, C6=5×, C7=1.5×) suggest electronic effects (Boger, D. L.; Yun, W. J. Am. Chem. Soc. 1994, 116, 5523-5524) modulated through the indole ring may attenuate activity but only to minor extent.

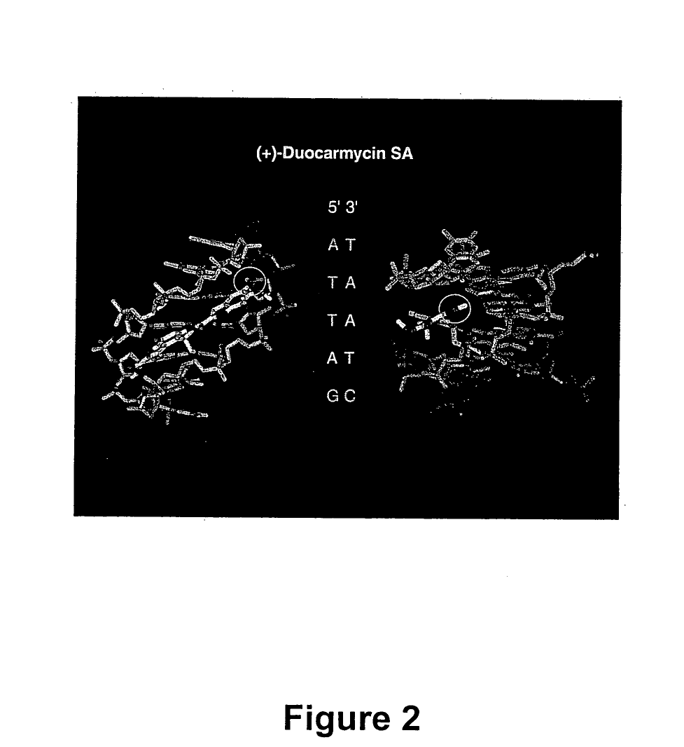

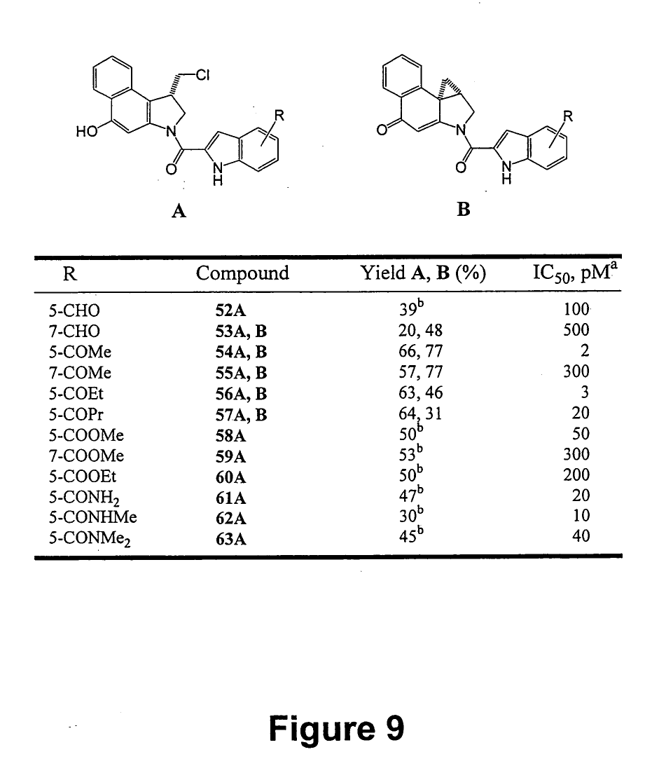

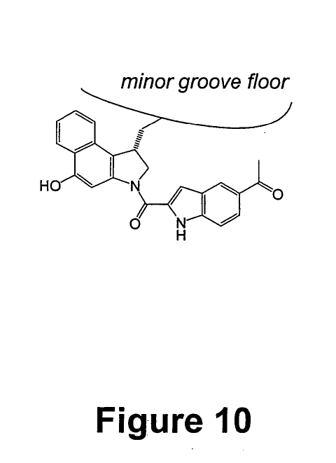

One of the most interesting series that was explored in detail and that provided exceptionally potent derivatives was the C5 indole derivatives bearing a carbonyl group directly attached to the indole, FIG. 9. Most significant of the observations was the behavior of the C5 acyl derivatives 54 and 56. A 1000-fold increase in potency over (+)-CBI-indole (5) was observed with 54 and 56 representing an additional ≧10-fold increase in potency beyond most C5 substituted derivatives described above. Analogues 54 and 56 are exceptionally potent cytotoxic compounds (IC₅₀=2-3 pM) exceeding the activity of CBI-TMI (4, 30 pM), CC-1065 (1, 20 pM), and duocarmycin SA (3, 6-10 pM). Interestingly, the analogue 57 containing a propyl chain reverted to the potency characteristic of the C5 substituted analogues (IC₅₀=20 pM), and did not show the further enhancement observed with 54 and 56. As discussed later, this may reflect the adoption of a DNA bound conformation for 54 and 56 analogous to the angular fusion of a 5-membered ring (see FIG. 10) with the methyl or ethyl group of 54 and 56 deeply embedded in the minor groove. This bound conformation would not be accessible to 57 because of the extended chain length and its behavior reverts back to that of an extended, flexible C5 substituent. The analogous, but less pronounced, enhancement observed with 54 and 56 may reflect a similar behavior. Notably, the methyl group of the C5 methoxy substituent of duocarmycin SA has been shown to extend into and be deeply embedded in the minor groove (Eis, P. S.; et al. J. Mol. Biol. 1997, 272, 237-252; Schnell, J. R.; et al. J. Am. Chem. Soc. 1999, 121, 5645-5652; Smith, J. A.; et al. J. Mol. Biol. 2000, 300, 1195-1204) consistent with such a bound conformation (FIG. 2).

Consistent with this special role for the methyl and ethyl groups of 54, 56, 58, and 60, the C5 formyl analogue 52, which would not benefit from such an interaction, proved to be 50-fold less potent. Although the corresponding ester derivatives 58 and 60 were significantly less potent with 58>60, the former, but not the latter, may benefit from an analogous minor groove embedded methoxy versus precluded ethoxy group with the weaker potency reflecting the less hydrophobic character of the interacting group. Finally, the corresponding carboxamide C5 derivatives 61-63, especially 61 and 62, were potent derivatives and it is tempting to suggest the enhanced potency of 62 benefits from a similar minor groove interaction. Notably, 62 was only 5-fold less potent than 54 and 3-fold more potent than (+)-CBI-TMI also placing it among the more potent derivatives examined.

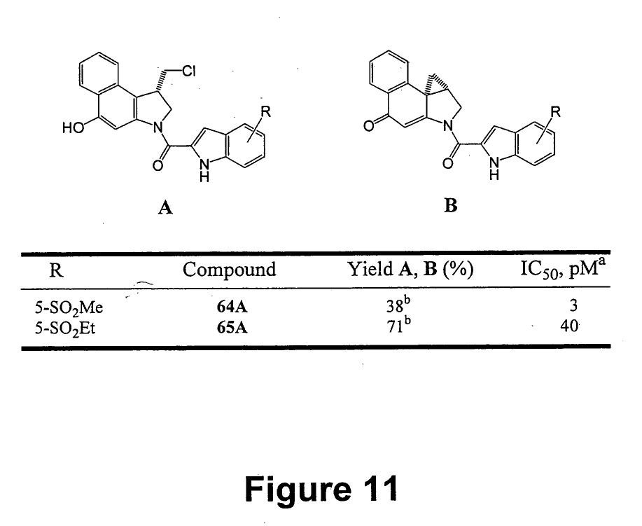

In an interesting extension of these observations, the sulfone derivatives 64 and 65 were examined, FIG. 11. Like 54, the C5 methanesulfonyl derivative 64 proved exceptionally potent (IC₅₀=3 pM) being 15-20 fold more active than the corresponding thiomethyl derivative 21 (IC₅₀=50 pM) or methoxy derivative 7 (IC₅₀=50 pM) and exceeding the activity of even 1-5. Unlike 56, the corresponding ethanesulfonyl derivative, while being a potent cytotoxic agent (IC₅₀=40 pM), did not exhibit this exceptional activity, and was only slightly more active than the corresponding thioethyl derivative 23 (IC₅₀=100 pM). This may reflect the larger size of S with 64, not 65, exhibiting shape characteristics closer to the potent ethyl ketone 54, and uniquely benefiting from the embedded minor groove interactions within the sulfone series.

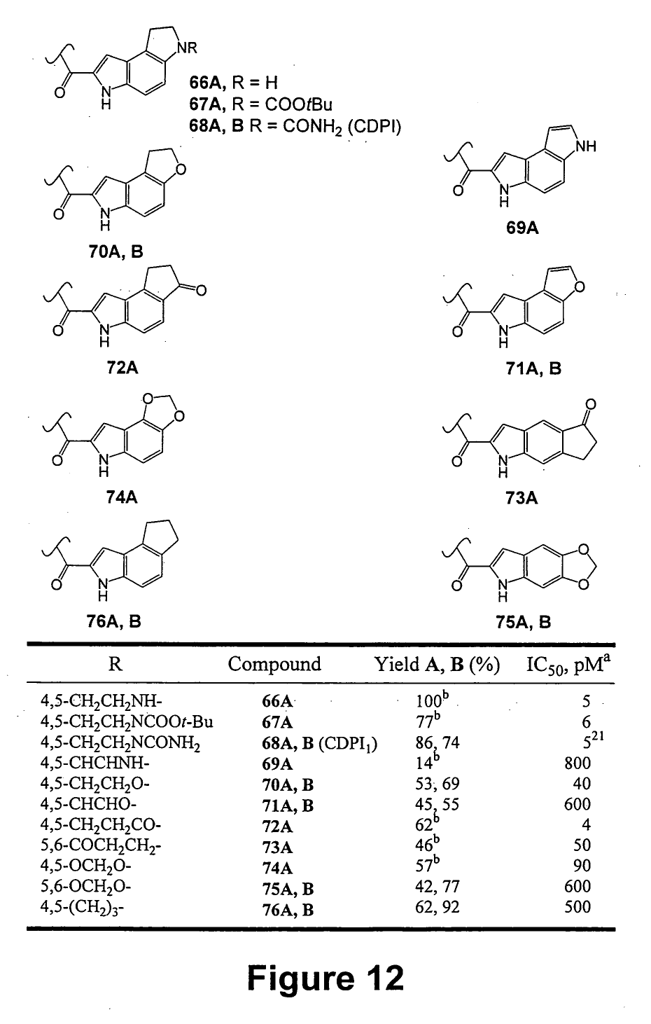

As a consequence of these observations, it was decided to reexamine tricyclic DNA binding subunits that bear structural characteristics of the potent indole derivatives disclosed herein. Prior studies disclosed (+)-CBI-CDPI₁ (68) embodying the structural characteristics of the DNA binding subunit of CC-1065 and showed that it was an exceptionally potent, simplified derivative (IC₅₀=5 pM) (Boger, D. L.; et al. J. Org. Chem. 1996, 61, 4894-4912). It contains a fused five-membered ring that may be regarded as a conformationally restricted and more rigid analogue of 44-48. The results of an examination of such alternative tricyclic systems that represent rigid or conformationally restricted analogues of the potent derivatives are summarized in FIG. 12. The cyclic structures representing substitutions at the 4,5 positions were expected to be superior to the 5,6 analogues due to their ability to adopt a conformation embodying the embedded minor groove substituent. Where examined, the 4,5-isomer was more potent than the corresponding 5,6-isomer (72 vs. 73 and 74 vs. 75), and introduction of unsaturation resulted in rather dramatic losses in activity (>10-fold, 66 vs. 69 and 71 vs. 70). Most importantly, the 4,5 constrained analogues (but not the 5,6 constrained analogues) typically matched or exceeded the potency of the corresponding unconstrained analogue. For example, 72, but not 73, matched the potency of the C5 substituted methyl and ethyl ketones 54 and 56. Similarly, 70 was slightly more potent than 7 (C5 OMe) and only slightly less potent than 15 (C5 OEt). Both CDPI and 67 were more potent than their corresponding C5 amide derivatives (40-48, IC₅₀=30-80 pM), and the fused cyclopentyl derivative 76 was more active than the corresponding C5 alkyl derivatives 24 and 25 (IC₅₀=2000 pM). Analogously, 74 was much more active than the corresponding unconstrained 4,5 dimethoxy derivative 10 whereas the 5,6 isomer 75 was substantially less potent than the corresponding unconstrained C5,6 dimethoxy derivative 11. Moreover, there was a pronounced difference in the relative potency of the constrained 4,5-derivatives that follows the trends observed with the analogous C5 substituent (—COR>—NCOR>—OR>—NR₂>—R where R=alkyl). Importantly, this series provided some of the most potent derivatives of CBI being 6-8 fold more potent than CBI-TMI (IC₅₀=30 pM) and ≧10-fold more potent than 5 (IC₅₀=50 pM) and surpassing the activity of CC-1065 (IC₅₀=20 pM) and duocarmycin SA (IC₅₀=6-10 pM).

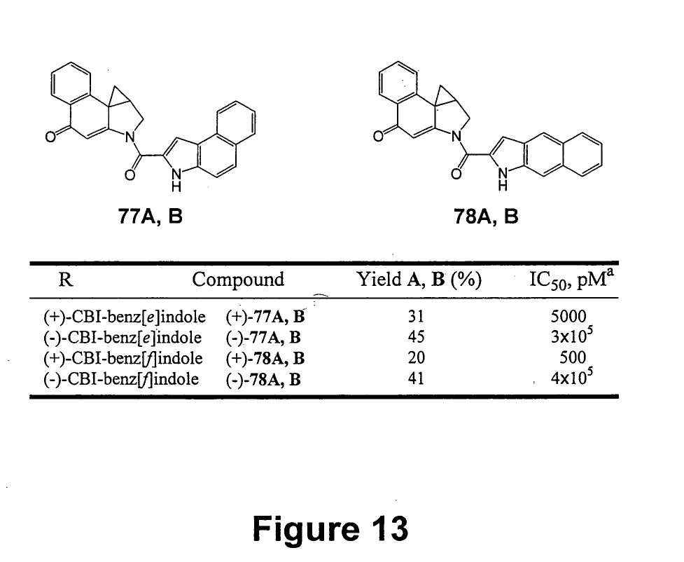

As shown in FIG. 13, extending the rigid length of the DNA binding indole by adding a linear or angular fused benzene ring did not substantially alter the cytotoxic potency. The linear extension with (+)-78 resulted in a modest 5-fold increase in potency whereas the angular extension with (+)-77 resulted in a modest 2-fold reduction when compared to (+)-CBI-indole (R═H) and a more significant 6-8 fold reduction relative to the fused pyrrole 69 or the fused furan 71. Consistent with expectations and past observations, the corresponding unnatural enantiomers (−)-77 and (−)-78 were found to be approximately 100-1000× less potent. The behavior of the angular derivatives (+)-77 and (−)-77 is especially striking in comparison with CBI-CDPI (68) and the related analogues in FIG. 12 which bear an angular fused saturated 5-membered ring. Presumably the increased sized and planar nature of the angularly fused benzene ring found in (+)-77 and (−)-77 hinders rather than facilitates minor groove binding and penetration required to observe DNA alkylation.

DNA Alkylation Selectivity, Efficiency, and Rate.

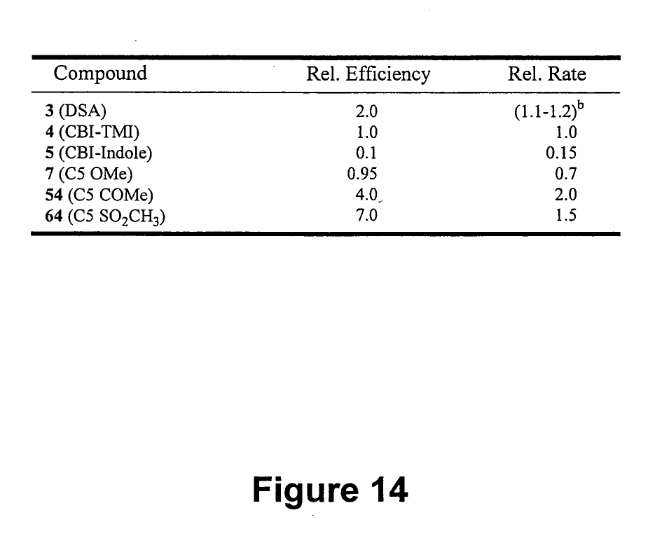

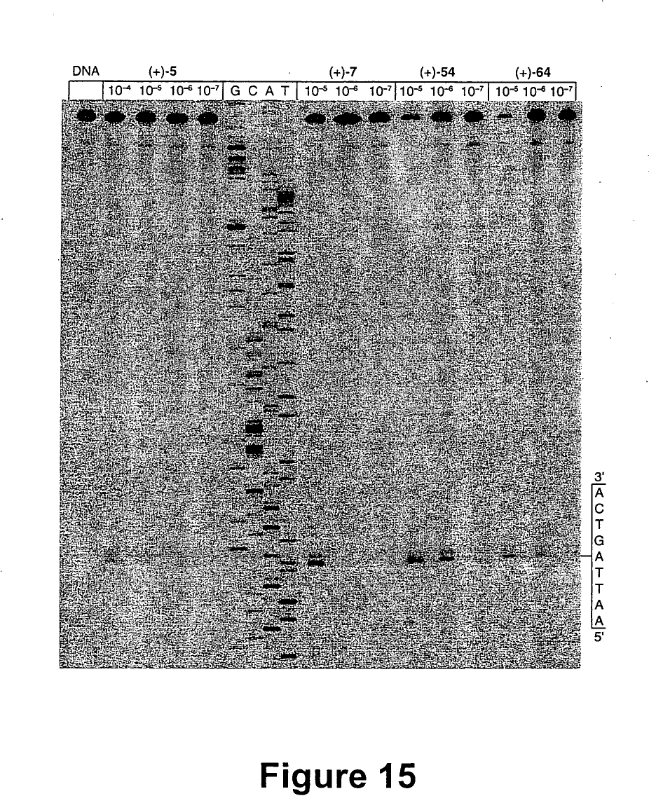

Although a number of features of 6-78 can contribute to distinctions observed in a cellular functional assay for cytotoxic activity, the most prominent feature characterized was the impact that the indole substituent had on the DNA alkylation properties. Thus, the DNA alkylation selectivity, efficiency, and rate of the simplest and most potent derivatives 54 and 64 were compared to duocarmycin SA (3), (+)-CBI-TMI (4), (+)-CBI-indole (5), and 7 in w794 DNA enlisting protocols previously introduced in earlier studies (Boger, D. L.; et al. Tetrahedron 1991, 47, 2661-2682). First, no alterations in the inherent DNA alkylation selectivity were observed with the new derivatives 54 and 64 consistent with past observations now entailing a wide range of CBI-based analogues (e.g. CBI-TMI vs. duocarmycin SA) (Boger, D. L.; et al. J. Am. Chem. Soc. 1994, 116, 1635-1656; Boger D. L.; Yun, W. J. Am. Chem. Soc. 1994, 116, 7996-8006). More importantly, the CBI analogues displayed significant distinctions in both their efficiencies in DNA alkylation (FIG. 15 and FIG. 14) and their rates of DNA alkylation (FIG. 14) that proved to correlate with the relative and absolute trends observed in their cytotoxic potency. Thus, in the case of the simplified and exceptionally potent derivatives 54 and 64, their enhanced cytotoxic potency correlates with an enhanced rate and efficiency of DNA alkylation.

C5 substituents on the first indole DNA binding subunit have a pronounced effect on the activity of CBI analogues of the duocarmycins and CC-1065. This effect, which provides as large as a 1000-fold increase in cytotoxic potency with 54 and 64 that correlate well with accompanying increases in the rate and efficiency of DNA alkylation, is more pronounced with the CBI versus DSA or CPI based analogues. Moreover, this effect is largely insensitive to the electronic character of the C5 substituent but is sensitive to the size, rigid length, and shape (sp, sp², sp³ hybridization) of this substituent consistent with expectation that the impact is due simply to its presence. With these substitutions, simplified CBI analogues were identified which surpass the potency of duocarmycin SA, CC-1065, and CBI-TMI. The comparison of these derivatives with a select set of conformationally constrained tricyclic indole derivatives suggest they additionally benefit from hydrophobic contacts in a pocket deeply imbedded in the minor groove. Thus, an indole C5 substituent may not only extend the rigid length of the compound enhancing the DNA binding induced disruption of the alkylation subunit vinylogous amide thereby accelerating the rate of DNA alkylation, but appropriate substituents may also benefit from stabilizing contacts within a minor groove hydrophobic pocket.

Materials

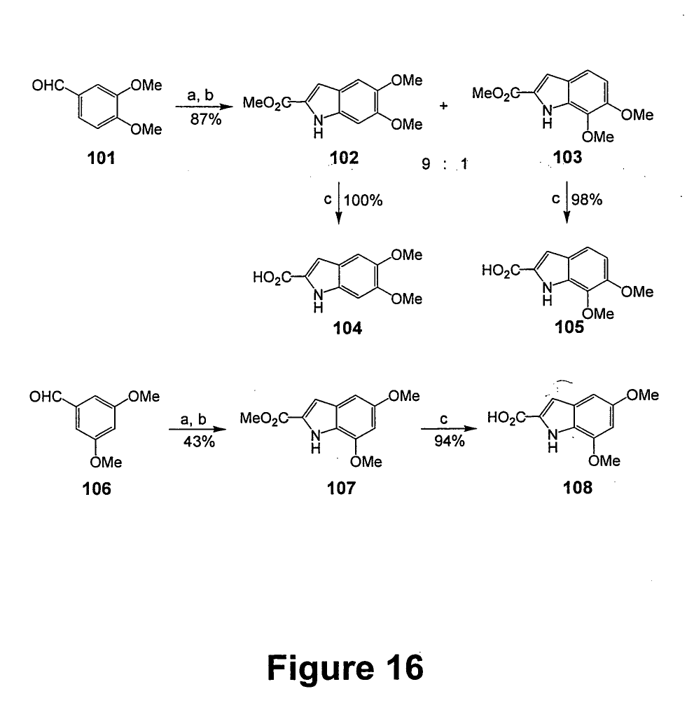

The 5,6-6,7- and 5,7-dimethoxyindole-2-carboxylic acids (104, 105, 108) were obtained by LiOH hydrolysis from the methyl esters, which were obtained by Hemetsberger indole synthesis starting from 3,4-dimethoxybenzaldehyde and 3,5-dimethoxybenzaldehyde, respectively (FIG. 16) (Reddy, M. S.; Cook, J. M. Tetrahedron Lett. 1994, 35, 5413-5416; De Antoni, A.; et al. Gazz. Chim. Ital. 1970, 100, 1056-1060; Crohare, R.; et al. J. Heterocycl. Chem. 1970, 7, 729-732).

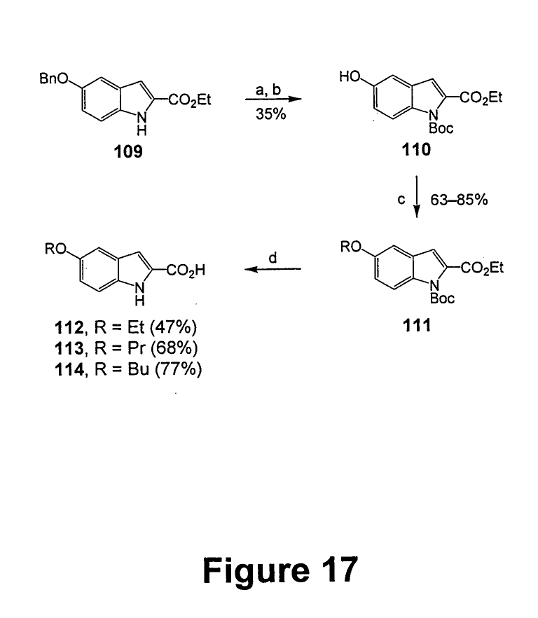

5-Ethoxyindole-2-carboxylic acid (112) was synthesized from ethyl 5-benzyloxyindole-2-carboxylate 109 by N-Boc protection of the indole, removal of the benzyl group with triethylsilane following Coleman's method, alkylation of the phenol and hydrolysis with KOH (FIG. 17) (Rydon, H. N.; Siddappa, S. J. Chem. Soc. 1951, 2462-2467; Coleman, R. S.; Shah, J. A. Synthesis 1999, 1399-1400). The same procedure was employed for 5-propyloxyindole-2-carboxylic acid (113) and 5-butyloxyindole-2-carboxylic acid (114).

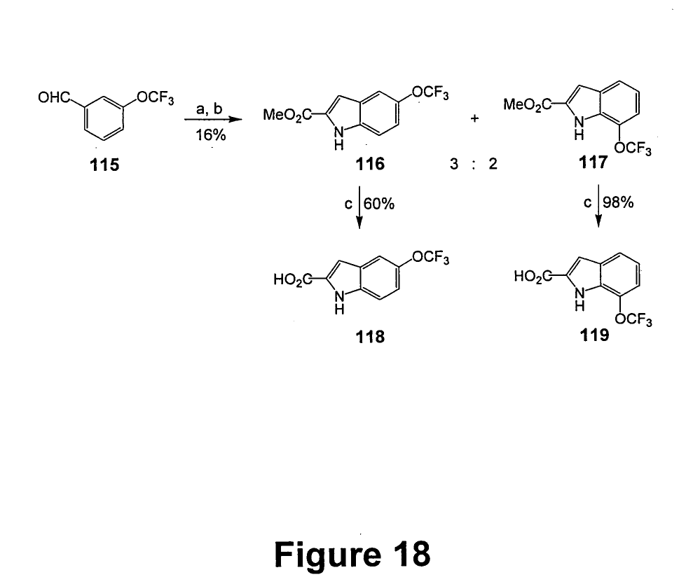

The 5- and 7-trifluoromethoxyindole-2-carboxylic acids (118, 119) were obtained by LiOH hydrolysis of the methyl esters (116, 117), obtained by the Hemetsberger reaction with 3-trifluoromethoxybenzaldehyde (FIG. 18) (Murakami, Y.; et al. Bull. Chem. Soc. Jpn. 1995, 43, 1281-1286).

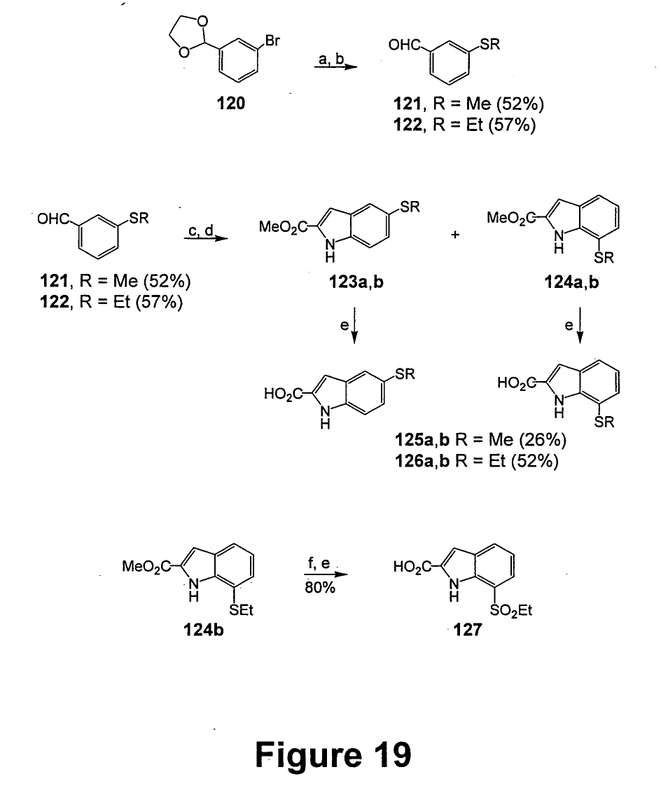

The 5- and 7-thiomethylindole-2-carboxylic acids (125, 126) and 5-thioethylindole-2-carboxylic acid (127) were obtained by the Hemetsberger reaction starting from the 3-thiomethyl-benzaldehyde and 3-thioethylbenzaldehyde, respectively (FIG. 19) (Patent: JP 2000 136182; Ornstein, P. L.; et al. J. Med. Chem. 1998, 41, 358-378; Watabe, T.; et al. J. Chem. Soc. Chem. Commun. 1983, 10, 585-586). These precursor compounds were prepared by treatment of 2-(3-bromophenyl)-[1,3]dioxolane 120 with BuLi followed by dimethyldisulfide or diethyldisulfide and acid mediated deprotection (Ornstein, P. L.; et al. J. Med. Chem. 1998, 41, 358-378). 5-Ethyl-sulfonylindole-2-carboxylic acid (127) was obtained by oxidation of 124 with m-CPBA and hydrolysis using LiOH.

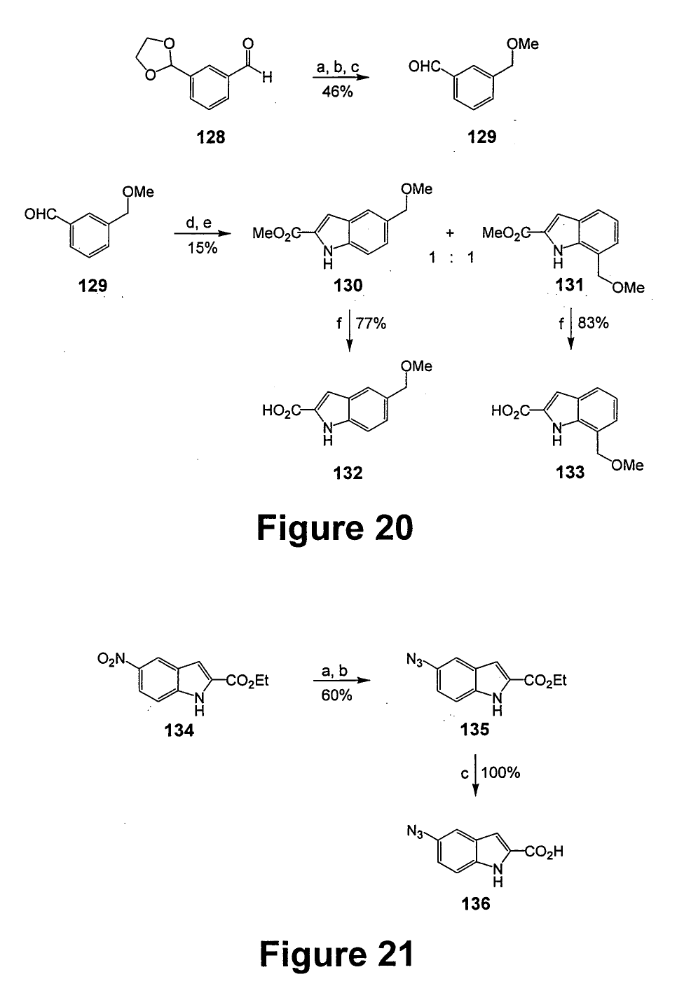

The 5- and 7-methoxymethylindole-2-carboxylic acids (132, 133) were obtained by hydrolysis with LiOH from the methyl esters (130, 131), prepared by the Hemetsberger procedure with the 3-methoxymethylbenzaldehyde (129) (Blaschke, H. J. Am. Chem. Soc. 1970, 92, 3675-3681). Aldehyde 129 was synthesized from 3-[1,3]dioxolan-2-yl-benzaldehyde (128) by reduction, methylation, and acid deprotection (FIG. 20).

5-Azidoindole-2-carboxylic acid (136) was synthesized from ethyl 5-nitroindole-2-carboxylate 134 following the method of Suschitzky (FIG. 21) (Scriven, E. F. V.; et al. J. Chem. Soc., Perkin Trans 1 1979, 53-59).

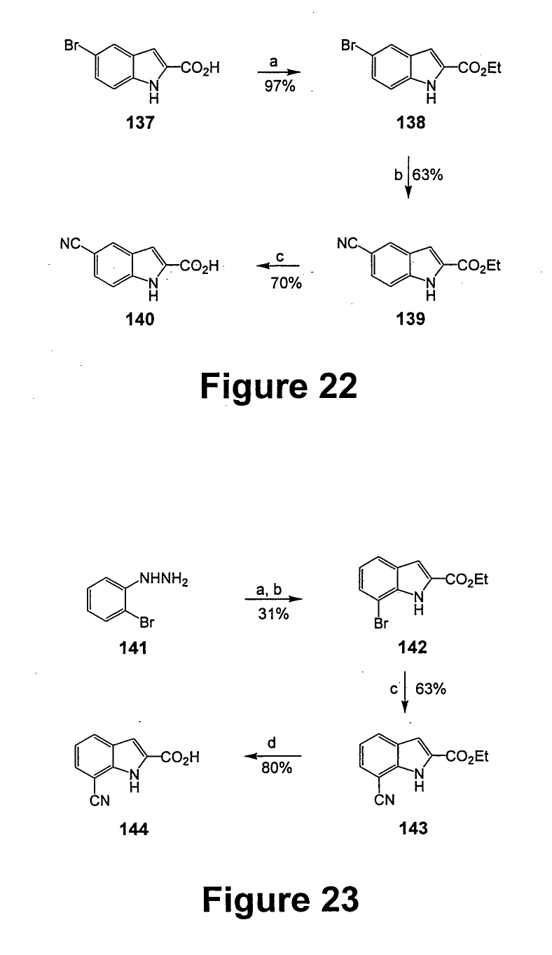

5-Cyanoindole-2-carboxylic acid (140) was synthesized using the method of Boger from ethyl 5-bromoindole-2-carboxylate 138 followed by hydrolysis using KOH (FIG. 22) (Lindwall, H. G.; Mantell, G. J. J. Org. Chem. 1953, 18, 345-354; Boger, D. L.; et al. J. Org. Chem. 1996, 61, 4894-4912).

7-Cyanoindole-2-carboxylic acid (144) was synthesized applying the same procedure as described for 140 starting with the ethyl 7-bromoindole-2-carboxylate 142 obtained by Fischer indole synthesis (FIG. 23) (Leggetter, B. E.; Brown, R. K. Can. J. Chem. 1960, 38, 1467-1471).

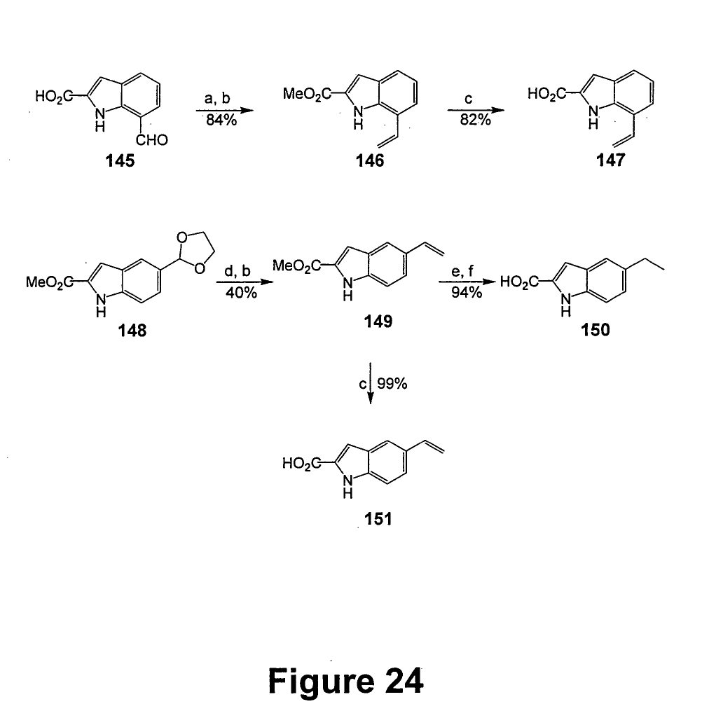

7-Vinylindole-2-carboxylic acid (147) was obtained by LiOH hydrolysis of the methyl ester 146, which was obtained by methylation of the acid 145 with diazomethane and Wittig olefination (FIG. 24). 5-Vinylindole-2-carboxylic acid 151 was obtained by hydrolysis of the methyl ester 159, obtained by acid deprotection of 148 followed by Wittig olefination. 5-Ethylindole-2-carboxylic acid (150) was obtained by hydrogenation with Pd/C of the olefin and hydrolysis with KOH.

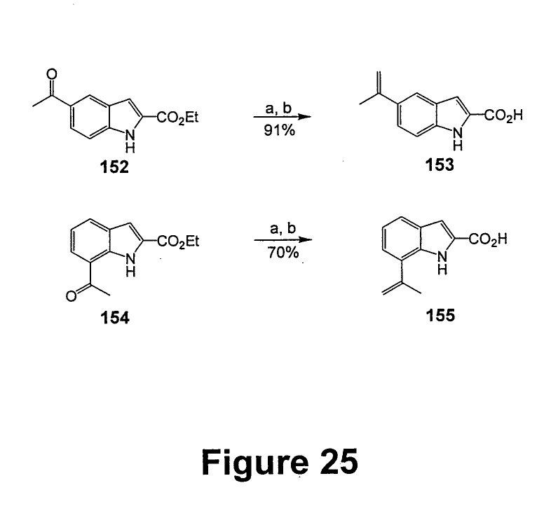

The 5- and 7-isopropenylindole-2-carboxylic acids (153 and 155) were obtained by LiOH hydrolysis of the methyl esters, obtained by Wittig olefination of the ketones 152 and 154 (FIG. 25).

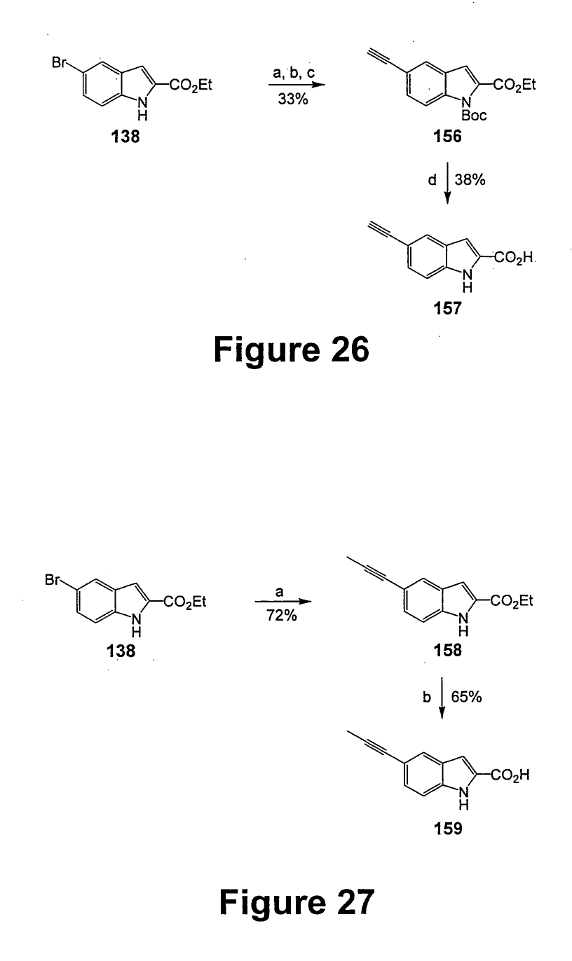

5-Ethynylindole-2-carboxylic acid (157) was obtained by hydrolysis of ethyl 5-ethynyl-N-tert-butyloxycarbonylindole-2-carboxylate 156, which was obtained from the corresponding bromide and trimethylsilylacetylene using the Fukuda method (59%) (Fukuda, Y.; et al. Bioorg. Med. Chem. Lett. 1998, 8, 1387-1390). The resulting mixture of product and starting material (17:3) was submitted to selective partial hydrolysis (trimethylsilyl deprotection) and N-Boc protection to allow the separation of 156 (FIG. 26).

5-(1-Propynyl)-indole-2-carboxylic acid (159) was obtained by treating ethyl 5-bromoindole-2-carboxylate 138 with propyne using a Pd(0)-Cu(I) coupling catalyst introduced by Buchwald and Fu and hydrolysis with LiOH (FIG. 27) (Hundertmark, T.; et al. Org. Lett. 2000, 2, 1729-1731).

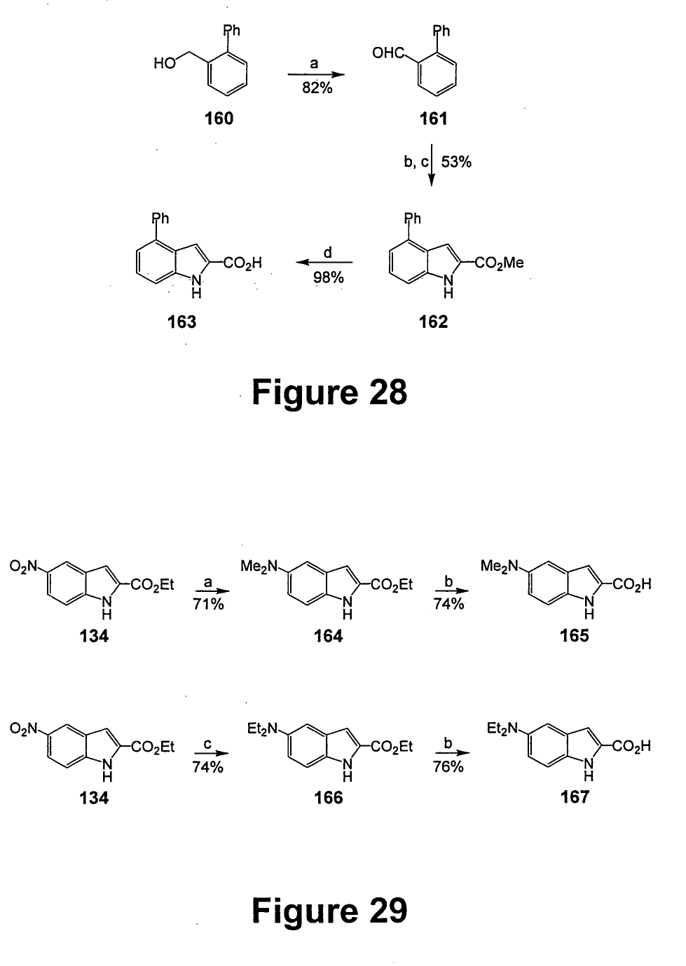

4-Phenylindole-2-carboxylic acid (163) was obtained by hydrolysis of the methyl ester 162 with KOH. The ester was obtained by the Hemetsberger indole synthesis, starting from 2-phenyl-benzaldehyde 161 which was obtained by oxidation of 2-phenylbenzylalcohol 160 (FIG. 28).

5-Dimethylaminoindole-2-carboxylic acid (165) was obtained by LiOH hydrolysis of ethyl 5-dimethylaminoindole indole-2-carboxylate 164. This material was prepared from corresponding ethyl 5-nitroindole-2-carboxylate (134) by a one-pot reduction and condensation with formaldehyde (FIG. 29). 5-Diethylaminoindole-2-carboxylic acid was prepared in the same manner.

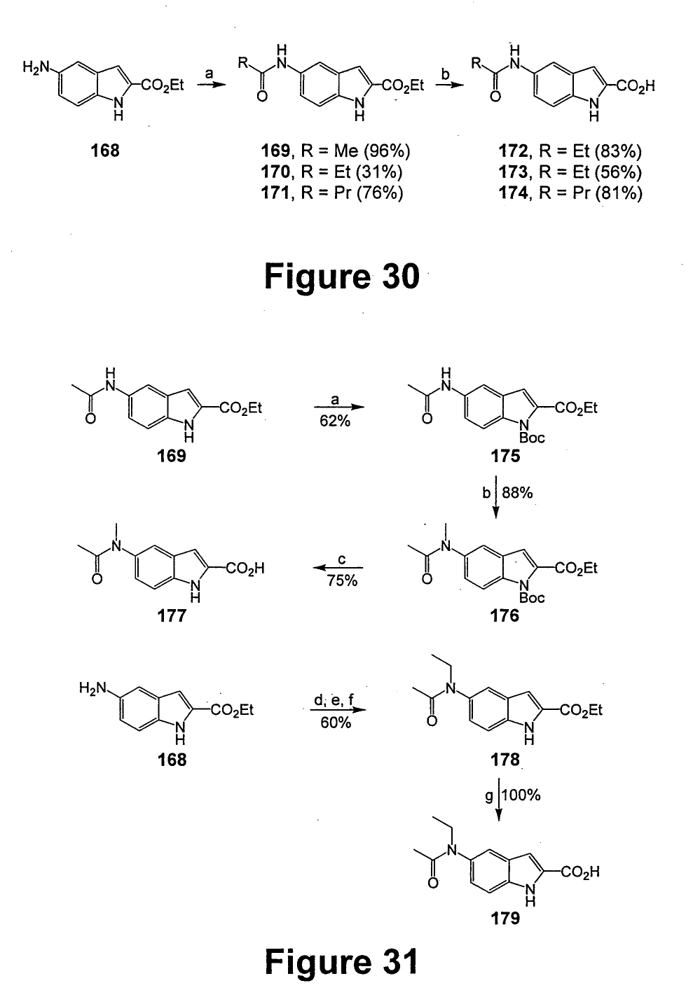

5-Aminoindole-2-carboxylic acid (168) was prepared by hydrogenation of the corresponding nitro group with 10% Pd/C in THF. 5-Acetylaminoindole-2-carboxylic acid (172) was obtained as previously reported by Denny by acylation and selective hydrolysis (FIG. 30) (Atwell, G. J.; et al. J. Med. Chem. 1999, 42, 3400-3411). The homologues 5-propionylaminoindole-2-carboxylic acid (173) and 5-butyrylaminoindole-2-carboxylic acid (174) were prepared in a similar fashion.

5-(N-Acetyl-N-methylamino)indole-2-carboxylic acid (177) was obtained from the ethyl ester by protecting the indole nitrogen with a Boc group, followed by MeI alkylation of the amide nitrogen and hydrolysis with LiOH. 5-(N-Acetyl-N-ethylamino)indole-2-carboxylic acid (179) was prepared by hydrolysis of the ethyl ester with Cs₂CO₃ (FIG. 31). To prepare this ester, the 5-amino compound 168 was converted to the diazonium salt and then followed by treatment with ethyl amine and acetic anhydride sequentially.

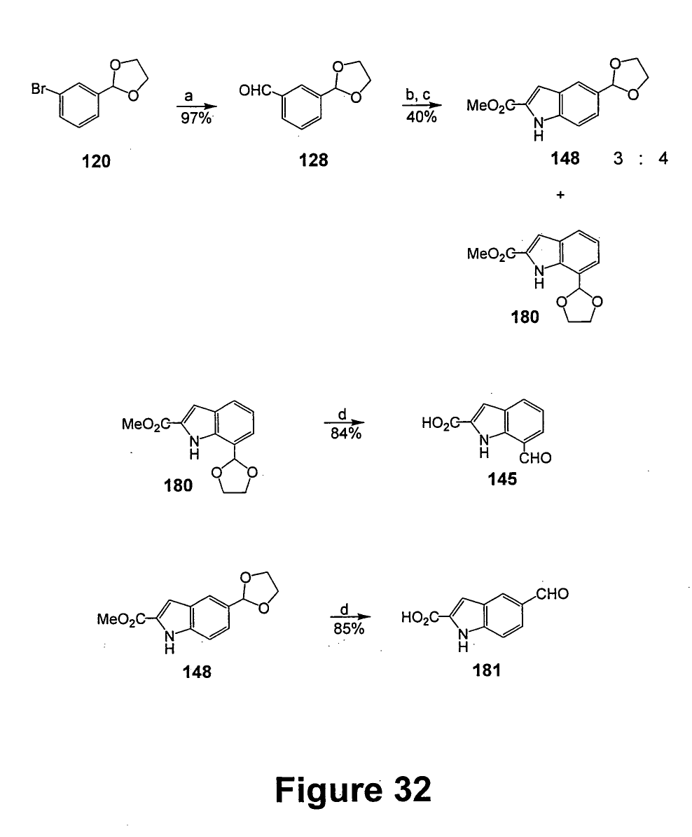

The 5- and 7-formylindole-2-carboxylic acids (145 and 181) were obtained by hydrolysis of the dioxolane protected methyl esters (148 and 180). These materials were prepared by the Hemetsberger reaction with 3-[1,3]dioxolan-2-ylbenzaldehyde 128, which was obtained from the 2-(3-bromophenyl)-[1,3]dioxolane 120 following a literature procedure (FIG. 32) (Marx, T.; Breitmaier, E. Liebigs Ann. Chem. 1992, 3, 183-186).

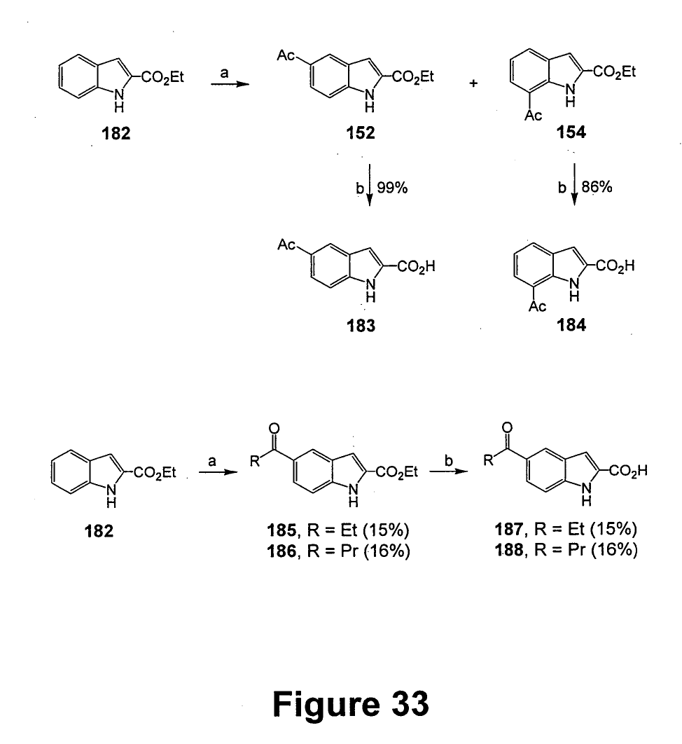

5-Acetylindole-2-carboxylic acid (183) and the 7-acetylindole-2-carboxylic acid (184) were synthesized by Friedel-Crafts acylation of ethyl indole-2-carboxylate 182 as reported by Murakami followed by hydrolysis with KOH (FIG. 33) (Avromenko, S. F. Chem. Heterocycl. Compd. 1970, 6, 1131; Murakami, Y.; et al. Heterocycles 1984, 22, 241-244). The same procedure was applied for the homologues 5-propionylindole-2-carboxylic acid (187) and 5-butyrylindole-2-carboxylic acid (188).

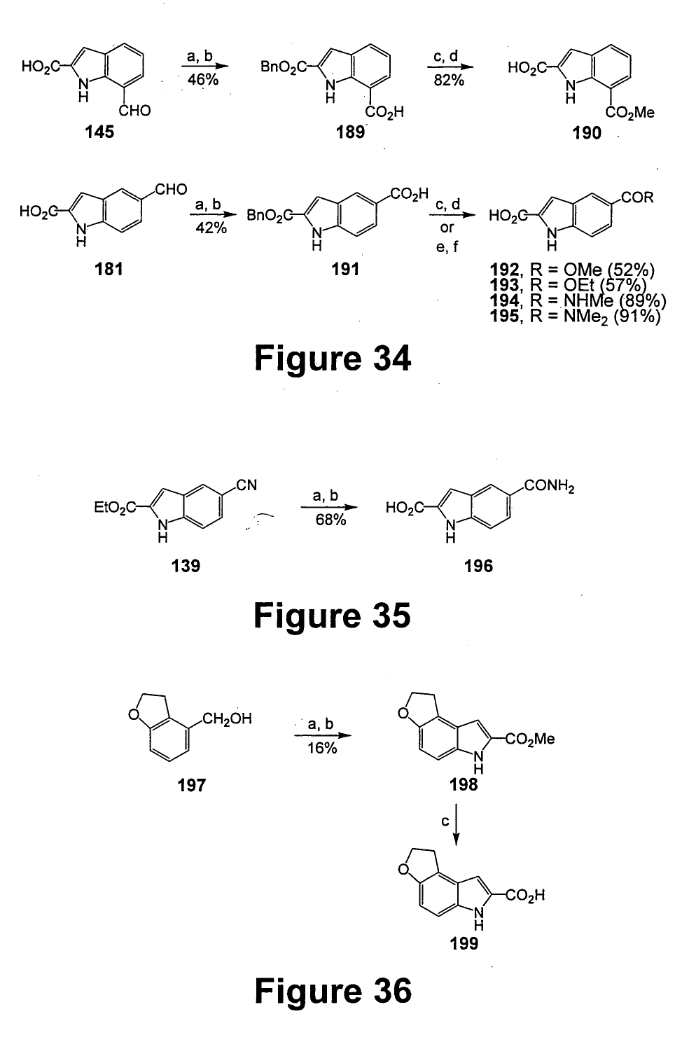

Several compounds were prepared from the same starting materials. For example, 145 and 181 were benzyl protected and oxidized to give 189 and 191, respectively (FIG. 34). Methylation with diazomethane and hydrogenolysis provided the 5- and 7-methoxycarbonylindole-2-carboxylic acids (190 and 192). In situ activation of the acid as the acyl chloride and treatment with a nucleophile (Nu=EtOH, MeNH₂ or Me₂NH), followed by hydrogenolysis gave 5-ethoxycarbonylindole-2-carboxylic acid (193), 5-methylcarbamoylindole-2-carboxylic acid (194) and 5-dimethylcarbamoylindole-2-carboxylic acid (195).

5-Carbamoylindole-2-carboxylic acid (196) was obtained by Cs₂CO₃ hydrolysis of the ethyl ester and selective hydrolysis of the cyano group (FIG. 35).

1,2-Dihydropyrrolo[3,2-e]benzofuran-7-carboxylic acid (199) was was obtained by hydrolysis of the methyl ester 198 (FIG. 36). This was obtained by the Hemetsberger reaction of the corresponding azido ester. The azido ester was prepared by condensation with commercially available aldehyde 198 and methyl α-azidoacetate. The aldehyde was prepared in six steps using the method of Eissenstat (Patent: PCT, U.S. 1998, 25829; Eissenstat, M. A.; et al. J. Med. Chem. 1995, 38, 3094-3105).

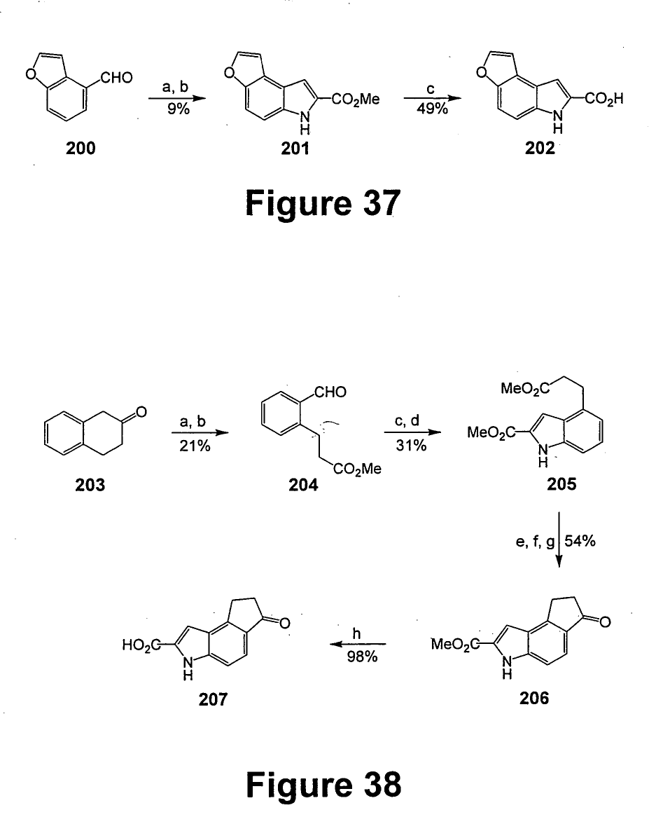

The same method of Eissenstat was used to prepare pyrrolo[3,2-e]benzofuran-7-carboxylic acid (202, FIG. 37) (Patent: PCT, U.S. 1998, 25829; Eissenstat, M. A.; et al. J. Med. Chem. 1995, 38, 3094-3105).

6-Oxo-cyclopenten[e]indole-2-carboxylic acid (207) was obtained by hydrolysis of the methyl ester 206, obtained by selective hydrolysis of the diester 205 followed by activation of the aliphatic acid as its acid chloride and intramolecular Friedel-Crafts acylation catalyzed by AlCl₃ (FIG. 38). Diester 205 was obtained by the Hemetsberger reaction starting from methyl 3-(2-formylphenyl)propionate 204. This was obtained from β-tetralone by ozonolysis of its methyl enolether (Zheng, Y.-J; Merz, Jr., K. M. J. Am. Chem. Soc. 1992, 114, 10498-10507; Guspanová, L.; et al. Helv. Chim. Acta 1997, 80, 1375).

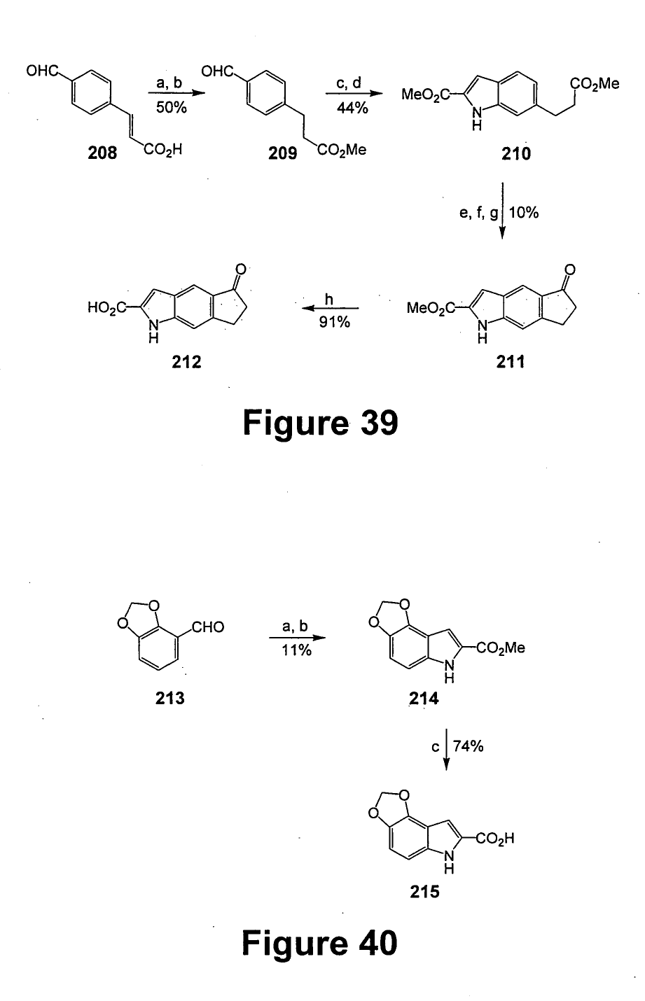

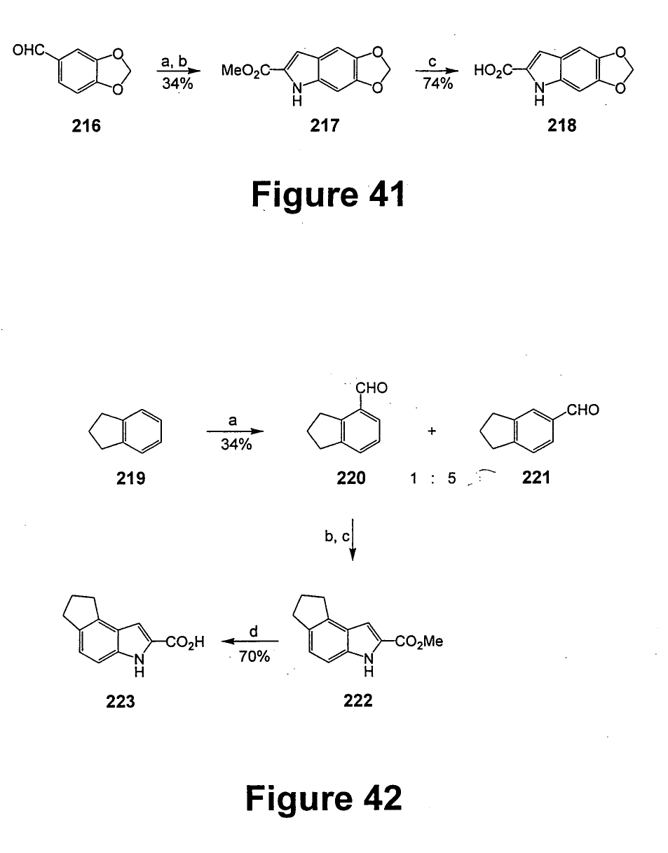

6-Oxo-cyclopenten[f]indole-2-carboxylic acid (212) was obtained by ester hydrolysis using LiOH. The ester was obtained by selective hydrolysis of the diester 210 followed by activation of the aliphatic acid as its acid chloride and intramolecular Friedel-Crafts acylation catalyzed by AlCl₃ (FIG. 39). Diester 210 was obtained by the Hemetsberger reaction starting from the methyl 3-(4-formylphenyl)propionate 209 (Matsuhashi, H.; et al. Bull. Chem. Soc. Jpn. 1997, 70, 437444). 209 was synthesized from 3-formylcinnamic acid by esterification with diazomethane and hydrogenation of the olefin in the presence of PtO₂.

[1,3]Dioxolo[e]indole-2-carboxylic acid (215) was obtained by hydrolysis of the methyl ester 214 (FIG. 40). This was obtained by the Hemetsberger reaction of the corresponding azido ester. The azido ester was prepared by condensation with commercially available aldehyde 213 and methyl α-azidoacetate.

[1,3]Dioxolo[f]indole-2-carboxylic acid (218) was obtained by hydrolysis of the methyl ester 217, which was obtained by the Hemetsberger reaction starting from the piperonal (FIG. 41) (Bu'Lock, J. D.; Harley-Mason, J. J. Chem. Soc., 1951, 703-712; Patent: WO 9109849).

Acid 223 was obtained by LiOH prompted hydrolysis of the methyl ester 222 (FIG. 42). This was obtained by the Hemetsberger reaction of the corresponding azido ester. The azido ester was prepared by condensation with aldehyde 219 and methyl α-azidoacetate. The aldehyde was obtained from treatment of indan with α,α-dichloromethyl methyl ether following the procedure of Mathison (Mathison, I. W.; et al. J. Org. Chem. 1974, 39, 2852-2855; Grunhaus, H.; et al. J. Heterocyclic. Chem. 1976, 13, 1161-1163).

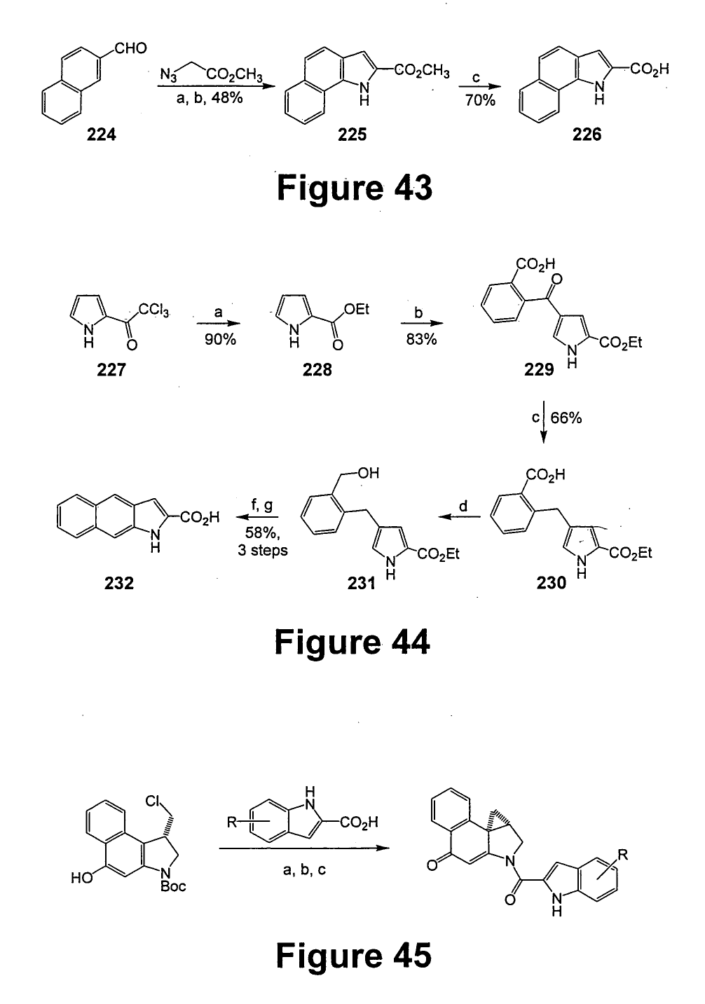

Benz[e]indole-2-carboxylic acid (226) was obtained by hydrolysis with KOH from the methyl ester 225 synthesized using the method of Macdonald (FIG. 43) (Beugelmans, R.; Chbani, M. Bull. Soc. Chim. Fr. 1995, 132, 729-733; Babushkina, T. A.; et al. J. Org. Chem. USSR 1975, 853-859; Miller, T. A.; et al. Bioorg. Med. Chem. Lett. 1998, 8, 1065-1070). Benz[f]indole-2-carboxylic acid (232) was obtained from 2-trichloroacetylpyrrole (FIG. 44) (Wallace, D. M.; et al. J. Org. Chem. 1993, 58, 7245-7257; Watanabe, T.; et al. J. Heterocycl. Chem. 1993, 30, 217-224; Murakami, Y.; Watanabe, T. J. Chem. Soc., Perkin Trans 1 1988, 3005-3012).

Methyl 4-methoxyindole-2-carboxylate, from which hydrolysis with KOH gave the 4-OMe acid, was initially prepared from the Hemetsberger indole synthesis protocol starting from the 2-methoxybenzaldehyde, but is currently available from Aldrich (Spadoni G.; et al. J. Med. Chem. 1998, 3624-3634; Allen, M. S.; et al. Synth. Commun. 1992, 22, 2077-2102). 7-Methoxy-indole-2-carboxylic acid was prepared as disclosed by Boger (Boger, D. L.; et al. J. Am. Chem. Soc. 1997, 119, 4977-4986). 4,5-Dimethoxy-2-carboxylic acid is available from ACB Block Ltd. or Ambinter. 5-hydroxyindole-2-carboxylic acid and 7-nitroindole-2-carboxylic acid were obtained from Acros. The esters 5-nitroindole-2-carboxylate and methyl 5-methylsulfonyl-indole-2-carboxylate were also commercially available, which after hydrolysis with LiOH gave the corresponding acids (Parmerter, S. M.; et al. J. Amer. Chem. Soc. 1958, 80, 4621). Additionally, 6-methoxyindole-2-carboxylic acid and 5-(N-t-butyloxycarbonylamino)indole-2-carboxylic acid are commercially available (Boger, D. L.; et al. J. Am. Chem. Soc. 1997, 119, 4977-4986; Boger, D. L.; et al. J. Am. Chem. Soc. 2000, 122, 6382-6394). Methyl 6-nitro-indole-2-carboxylate and methyl 3,6-dihydropyrrolo[e]indole-2-carboxylate are also available commercially and the acids prepared by LiOH hydrolysis (Allen, M. S.; et al. Synth. Commun. 1992, 22, 2077-2102; Faucher, N. 2001, personal communication; Boger, D. L.; et al. J. Org. Chem. 1987, 53, 1521-1530). (N-tert-Butyloxycarbonyl)-3,6,7,8-tetrahydropyrrolo[e]indole-2-carboxylic acid was prepared according to Boger (Boger, D. L.; et al. J. Org. Chem. 1987, 53, 1521-1530).

To prepare the agents, the CBI alkylation subunit was coupled with the desired indole-2-carboxylic acid by deprotection of seco-N-Boc-CBI with 4 N HCl/EtOAc (2 h, 23° C., >99%) and then treatment with EDCl in absence of base (FIG. 45). The spirocyclization was achieved using DBU in CH₃CN or CH₃CN/THF. In general, the seco-agents bearing an electron withdrawing group did not spirocyclize as well as the agents containing electron donating groups and consequently spirocyclization was not achieved for the majority of these agents.

Experimental

General Procedure for the Synthesis of the Seco Agents:

A solution of the natural enantiomer of seco-N-Boc-CBI (2.5 mg, 7.5 μmol) in 1 mL of 4 M HCl (EtOAc) was stirred for 1 h at 25° C. The solvent was then removed under a stream of N₂. The residue was dried under high vacuum for 3 h and the indole-2-carboxylic acid (8.3 μmol) was added. A solution of EDCl (4.3 μmol) in 200 μL of DMF was added and the reaction mixture was stirred for 14 h at 25° C. The reaction mixture was then diluted with CH₂Cl₂ (1 mL) and the solvent was removed under a stream of N₂. The product was purified by preparative TLC (10×20 cm, CH₂Cl₂/acetone 1:1 or THF/hexane 1:1 to 3:1).

3-(4-Methoxyindole-2-carbonyl)-1-(S)-(chloromethyl)-5-hydroxy-1,2-dihydro-3H-benz[e]indole (6A): (1.44 mg, 47%) as a off-white solid: [α]²³_{D} +33 (c 0.2, THF); MALDIFT-HRMS m/z 407.1162 (M+H⁺, C₂₃H₂₀ClN₂O₃ requires 407.1157).

3-(5-Methoxyindole-2-carbonyl)-1-(S)-(chloromethyl)-5-hydroxy-1,2-dihydro-3H-benz[e]indole (7A): (2.30 mg, 75%) as a white solid: [α]²³_{D} +14 (c 0.13, THF); MALDIFT-HRMS m/z 406.1070 (M⁺, C₂₃H₁₉ClN₂O₃ requires 406.1084).

3-(6-Methoxyindole-2-carbonyl)-1-(S)-(chloromethyl)-5-hydroxy-1,2-dihydro-3H-benz[e]indole (8A): (1.63 mg, 53%) as a pale yellow solid: [α]²³_{D} −45 (c 0.07, THF); MALDIFT-HRMS m/z 406.1093 (M⁺, C₂₃H₁₉ClN₂O₃ requires 406.1079).

3-(7-Methoxyindole-2-carbonyl)-1-(S)-(chloromethyl)-5-hydroxy-1,2-dihydro-3H-benz[e]indole (9A): (1.59 mg, 52%) as a pale yellow solid: [α]²³_{D} +1 (c 0.2, THF); MALDIFT-HRMS m/z 407.1170 (M+H⁺, C₂₃H₁₉ClN₂O₂ requires 407.1157).

3-(4,5-Dimethoxyindole-2-carbonyl)-1-(S)-(chloromethyl)-5-hydroxy-1,2-dihydro-3H-benz[e]indole (10A): (1.9 mg, 48%) as a white solid: [α]²³_{D} +43 (c 0.03, acetone); MALDIFT-HRMS m/z 437.1276 (M+H⁺, C₂₄H₂₁ClN₂O₄ requires 437.1263).

3-(5,6-Dimethoxyindole-2-carbonyl)-1-(S)-(chloromethyl)-5-hydroxy-1,2-dihydro-3H-benz[e]indole (11A): (2.01 mg, 61%) as a pale yellow solid: [α]²³_{D} +11 (c 0.1, THF); MALDIFT-HRMS m/z 436.1179 (M⁺, C₂₄H₂₁ClN₂O₄ requires 436.1184).

3-(6,7-Dimethoxyindole-2-carbonyl)-1-(S)-(chloromethyl)-5-hydroxy-1,2-dihydro-3H-benz[e]indole (12A): (1.41 mg, 43%) as a pale yellow solid: [α]²³_{D} +9 (c 0.07, THF); MALDIFT-HRMS m/z 437.1271 (M+H⁺, C₂₄H₂₂ClN₂O₄ requires 437.1263).

3-(5,7-Dimethoxyindole-2-carbonyl)-1-(S)-(chloromethyl)-5-hydroxy-1,2-dihydro-3H-benz[e]indole (13A): (1.81 mg, 55%) as an off-white solid: [α]²³_{D} +11 (c 0.1, THF); MALDIFT-HRMS m/z 437.1259 (M+H⁺, C₂₄H₂₂ClN₂O₄ requires 437.1263).

3-(5-Hydroxyindole-2-carbonyl)-1-(S)-(chloromethyl)-5-hydroxy-1,2-dihydro-3H-benz[e]indole (14A): (1.27 mg, 43%) as an off-white solid: [α]²³_{D} +11 (c 0.06, THF); MALDIFT-HRMS m/z 393.0997 (M+H⁺, C₂₂H₁₈ClN₂O₃ requires 393.1000).

3-(5-Ethoxyindole-2-carbonyl)-1-(S)-(chloromethyl)-5-hydroxy-1,2-dihydro-3H-benz[e]indole (15A): (1.71 mg, 54%) as a white solid: [α]²³_{D} +19 (c 0.1, THF); MALDIFT-HRMS m/z 421.1295 (M+H⁺, C₂₄H₂₂ClN₂O₃ requires 421.1313).

3-(5-Propyloxyindole-2-carbonyl)-1-(S)-(chloromethyl)-5-hydroxy-1,2-dihydro-3H-benz[e]indole (16A): (2.06 mg, 63%) as a beige solid: [α]²³_{D} +10 (c 0.2, THF); MALDIFT-HRMS m/z 434.1404 (M⁺, C₂₅H₂₃ClN₂O₃ requires 434.1397).

3-(5-Butyloxyindole-2-carbonyl)-1-(S)-(chloromethyl)-5-hydroxy-1,2-dihydro-3H-benz[e]indole (17A): (1.53 mg, 46%) as a beige solid: [α]²³_{D} +20 (c 0.1, THF); MALDIFT-HRMS m/z 449.1611 (M+H⁺, C₂₆H₂₆ClN₂O₃ requires 449.1626).

3-(5-Benzyloxyindole-2-carbonyl)-1-(S)-(chloromethyl)-5-hydroxy-1,2-dihydro-3H-benz[e]indole (18A): (1.12 mg, 31%) as a white solid: [α]²³₀+36 (c 0.06, THF); MALDIFT-HRMS m/z 483.1466 (M+H⁺, C₂₉H₂₄ClN₂O₃ requires 483.1470).

3-(5-Trifluoromethoxyindole-2-carbonyl)-1-(S)-(chloromethyl)-5-hydroxy-1,2-dihydro-3H-benz[e]indole (19A): (1.83 mg, 53%) as a beige solid: [α]²³_{D} +9 (c 0.1, THF);

3-(7-Trifluoromethoxyindole-2-carbonyl)-1-(S)-(chloromethyl)-5-hydroxy-1,2-dihydro-3H-benz[e]indole (20A): (2.26 mg, 65%) as a pale yellow solid: [α]²³_{D} −7 (c 0.1, THF); MALDIFT-HRMS m/z 460.0799 (M⁺, C₂₃H₁₆ClF₃N₂O₃ requires 460.0796).

3-(5-Thiomethylindole-2-carbonyl)-1-(S)-(chloromethyl)-5-hydroxy-1,2-dihydro-3H-benz[e]indole (21A): (1.23 mg, 39%) as a yellow solid: [α]²³_{D} +28 (c 0.05, THF); MALDIFT-HRMS m/z 422.0845 (M⁺, C₂₃H₁₉ClN₂O₂S requires 422.0850).

3-(7-Thiomethylindole-2-carbonyl)-1-(S)-(chloromethyl)-5-hydroxy-1,2-dihydro-3H-benz[e]indole (22A): (1.74 mg, 55%) as a yellow solid: [α]²³_{D} −19 (c 0.08, THF); MALDIFT-HRMS m/z 422.0855 (M⁺, C₂₃H₁₉ClN₂O₂S requires 422.0850).

3-(5-Thioethylindole-2-carbonyl)-1-(S)-(chloromethyl)-5-hydroxy-1,2-dihydro-3H-benz[e]indole (23A): (2.02 mg, 62%) as a pale yellow solid: [α]²³_{D} +8 (c 0.1, THF); MALDIFT-HRMS m/z 437.1086 (M+H⁺, C₂₄H₂₂ClN₂O₂S requires 437.1085).

3-(Indole-2-carbonyl)-1-(S)-(chloromethyl)-5-hydroxy-1,2-dihydro-3H-benz[e]indole (5A, CBI-indole): (1.53 mg, 57%) as a white solid: [α]²³_{D} +10 (c 0.08, THF); MALDIFT-HRMS m/z 376.0987 (M⁺, C₂₂H₁₇ClN₂O₂ requires 376.0978).

3-(5-Methylindole-2-carbonyl)-1-(S)-(chloromethyl)-5-hydroxy-1,2-dihydro-3H-benz[e]indole (24A): (1.55 mg, 53%) as a white solid: [α]²³_{D} +6 (c 0.1, THF); MALDIFT-HRMS m/z 390.1149 (M⁺, C₂₃H₁₉ClN₂O₂ requires 390.1135).

3-(5-Ethylindole-2-carbonyl)-1-(S)-(chloromethyl)-5-hydroxy-1,2-dihydro-3H-benz[e]indole (25A): (1.35 mg, 44%) as a beige solid: [α]²³_{D} +20 (c 0.07, THF); MALDIFT-HRMS m/z 405.1359 (M+H⁺, C₂₄H₂₂ClN₂O₂ requires 405.1364).

3-(5-Methoxymethylindole-2-carbonyl)-1-(S)-(chloromethyl)-5-hydroxy-1,2-dihydro-3H-benz[e]indole (26A): (1.66 mg, 53%) as a beige solid: [α]²³_{D} +3 (c 0.08, THF); MALDIFT-HRMS m/z 421.1332 (M+H⁺, C₂₄H₂₂ClN₂O₃ requires 421.1313).

3-(7-Methoxymethylindole-2-carbonyl)-1-(S)-(chloromethyl)-5-hydroxy-1,2-dihydro-3H-benz[e]indole (27A): (1.40 mg, 44%) as a pale yellow solid: [α]²³_{D} −9 (c 0.07, THF); MALDIFT-HRMS m/z 420.1255 (M⁺, C₂₄H₂₁ClN₂O₃ requires 420.1235).

3-(5-Bromoindole-2-carbonyl)-1-(S)-(chloromethyl)-5-hydroxy-1,2-dihydro-3H-benz[e]indole (28A): (1.90 mg, 56%) as a beige solid: [α]²³_{D} +39 (c 0.06, THF); MALDIFT-HRMS m/z 454.0085 (M⁺, C₂₂H₁₆BrClN₂O₂ requires 454.0084).

3-(5-Chloroindole-2-carbonyl)-1-(S)-(chloromethyl)-5-hydroxy-1,2-dihydro-3H-benz[e]indole (29A): (2.28 mg, 74%) as a beige solid: [α]²³_{D} +22 (c 0.13, THF); MALDIFT-HRMS m/z 410.0594 (M⁺, C₂₂H₁₆Cl₂N₂O₂ requires 410.0583).

3-(5-Azidoindole-2-carbonyl)-1-(S)-(chloromethyl)-5-hydroxy-1,2-dihydro-3H-benz[e]indole (30A): (1.88 mg, 60%) as a beige solid: [α]²³₀+30 (c 0.07, THF); MS (ESI negative) m/z 416 (M−H—).

3-(5-Cyanoindole-2-carbonyl)-1-(S)-(chloromethyl)-5-hydroxy-1,2-dihydro-3H-benz[e]indole (31A): (2.12 mg, 70%) as a beige solid: [α]²³_{D} +38 (c 0.08, THF); MALDIFT-HRMS m/z 401.0927 (M⁺, C₂₃H₁₆ClN₃O₂ requires 401.0931).

3-(7-Cyanoindole-2-carbonyl)-1-(S)-(chloromethyl)-5-hydroxy-1,2-dihydro-3H-benz[e]indole (32A): (1.04 mg, 35%) as a yellow solid: [α]²³_{D} −12 (c 0.05, THF); MALDIFT-HRMS m/z 402.1017 (M+H⁺, C₂₃H₁₇ClN₃O₂ requires 402.1004).

3-(5-Vinylindole-2-carbonyl)-1-(S)-(chloromethyl)-5-hydroxy-1,2-dihydro-3H-benz[e]indole (33A): (1.62 mg, 54%) as a pale yellow solid: [α]²³_{D} +33 (c 0.08, THF); MALDIFT-HRMS m/z 403.1220 (M+H⁺, C₂₄H₂₀ClN₂O₂ requires 403.1208).

3-(7-Vinylindole-2-carbonyl)-1-(S)-(chloromethyl)-5-hydroxy-1,2-dihydro-3H-benz[e]indole (34A): (1.24 mg, 41%) as a pale yellow solid: [α]²³_{D} −12 (c 0.07, THF); ESI (negative) m/z 401 (M−H⁻, C₂₄H₁₈ClN₂O₂).

3-(5-Isopropenylindole-2-carbonyl)-1-(S)-(chloromethyl)-5-hydroxy-1,2-dihydro-3H-benz[e]indole (35A): (1.58 mg, 51%) as a yellow solid: [α]²³_{D} +60 (c 0.07, THF); ESI (negative) m/z 415 (M−H⁻, C₂₅H₂₀ClN₂O₂).

3-(7-Isopropenylindole-2-carbonyl)-1-(S)-(chloromethyl)-5-hydroxy-1,2-dihydro-3H-benz[e]indole (36A): (1.99 mg, 64%) as a white solid: [α]²³_{D} −10 (c 0.1, THF); ESI (positive) m/z 439 (M+Na⁺, C₂₅H₂₁ClN₂NaO₂), 417 (M+H⁺, C₂₅H₂₂ClN₂O₂); ESI (negative) m/z 415 (M−H⁻, C₂₅H₂₀ClN₂O₂).

3-(5-Ethynylindole-2-carbonyl)-1-(S)-(chloromethyl)-5-hydroxy-1,2-dihydro-3H-benz[e]indole (37A): (1.00 mg, 33%) as a beige solid: [α]²³_{D} +37 (c 0.1, THF); MALDIFT-HRMS m/z 401.1051 (M+H⁺, C₂₄H₁₈ClN₂O₂ requires 401.1051).

3-[5-(1-Propynyl)indole-2-carbonyl]-1-(S)-(chloromethyl)-5-hydroxy-1,2-dihydro-3H-benz[e]indole (38A): (1.02 mg, 33%) as a beige solid: [α]²³_{D} +39 (c 0.07, THF); MALDIFT-HRMS m/z 415.1203 (M+H⁺, C₂₅H₂₀ClN₂O₂ requires 415.1208).

3-(4-Phenylindole-2-carbonyl)-1-(S)-(chloromethyl)-5-hydroxy-1,2-dihydro-3H-benz[e]indole (39A): (1.42 mg, 42%) as a off-white solid: [α]²³_{D} +93 (c 0.07, THF); MALDIFT-HRMS m/z 452.1288 (M⁺, C₂₈H₂₁ClN₂O₂ requires 452.1286).

3-(5-Aminoindole-2-carbonyl)-1-(S)-(chloromethyl)-5-hydroxy-1,2-dihydro-3H-benz[e]indole (40A): Prepared by treatment of 43A (1.0 mg, 1.9 μmol) with 4 N HCl/EtOAc (500 μL) for 1 h at 23° C., followed by solvent removal to afford 40A (750 μg, 87%) as a pale yellow solid: ESI (positive) m/z 392 (M+H⁺, C₂₂H₁₉ClN₃O₂); ESI (negative) m/z 390 (M−H⁻, C₂₂H₁₇ClN₃O₂).

3-(5-Dimethylaminoindole-2-carbonyl)-1-(S)-(chloromethyl)-5-hydroxy-1,2-dihydro-3H-benz[e]indole (41A): (2.40 mg, 23%) as a yellow solid: [α]²³_{D} +15 (c 0.026, CH₂Cl₂); MALDIFT-HRMS m/z 420.1471 (M+H⁺, C₂₄H₂₃ClN₃O₂ requires 420.1473).

3-(5-Diethylaminoindole-2-carbonyl)-1-(S)-(chloromethyl)-5-hydroxy-1,2-dihydro-3H-benz[e]indole (42A): (2.80 mg, 20%) as a yellow solid: [α]²³_{D} +33 (c 0.015, CH₂Cl₂); MALDIFT-HRMS m/z 420.1471 (M+H⁺, C₂₄H₂₃ClN₃O₂ requires 420.1473).

3-(5-t-Butyloxycarbonylaminoindole-2-carbonyl)-1-(S)-(chloromethyl)-5-hydroxy-1,2-dihydro-3H-benz[e]indole (43A): (2.02 mg, 55%) as a pale yellow solid: [α]²³_{D} +40 (c 0.02, THF); MALDIFT-HRMS m/z 514.1501 (M+Na⁺, C₂₇H₂₆ClN₃NaO₄ requires 514.1504).

3-(5-Acetylaminoindole-2-carbonyl)-1-(S)-(chloromethyl)-5-hydroxy-1,2-dihydro-3H-benz[e]indole (44A): (0.89 mg, 27%) as a white solid: [α]²³_{D} +21 (c 0.033, THF); MALDIFT-HRMS m/z 434.1269 (M+H⁺, C₂₄H₂₁ClN₃O₃ requires 434.1266).

3-(5-Propionylaminoindole-2-carbonyl)-1-(S)-(chloromethyl)-5-hydroxy-1,2-dihydro-3H-benz[e]indole (45A): (1.00 mg, 30%) as a pale yellow solid: [α]²³_{D} +24 (c0.05, THF); MALDIFT-HRMS m/z 447.1350 (M⁺, C₂₅H₂₂ClN₃O₃ requires 447.1350).

3-(5-Butyrylaminoindole-2-carbonyl)-1-(S)-(chloromethyl)-5-hydroxy-1,2-dihydro-3H-benz[e]indole (46A): (1.51 mg, 44%) as a white solid: [α]²³_{D} +43 (c 0.08, THF); MALDIFT-HRMS m/z 462.1565 (M⁺, C₂₆H₂₄ClN₃O₃ requires 462.1579).

3-[5-(N-Acetyl-N-methyl)aminoindole-2-carbonyl]-1-(S)-(chloromethyl)-5-hydroxy-1,2-dihydro-3H-benz[e]indole (47A): (1.60 mg, 48%) as a pale yellow solid: [α]²³_{D} +19 (c 0.2, THF); MALDIFT-HRMS m/z 448.1420 (M+H⁺, C₂₅H₂₃ClN₃O₃ requires 448.1420).

3-[5-(N-Acetyl-N-ethyl)aminoindole-2-carbonyl]-1-(S)-(chloromethyl)-5-hydroxy-1,2-dihydro-3H-benz[e]indole (48A): (0.60 mg, 39%) as a pale yellow solid: [α]²³_{D} +35 (c 0.08, acetone); MALDIFT-HRMS m/z 462.1597 (M+H⁺, C₂₆H₂₄ClN₃O₃ requires 462.1579).

3-(5-Nitroindole-2-carbonyl)-1-(S)-(chloromethyl)-5-hydroxy-1,2-dihydro-3H-benz[e]indole (49A): (1.55 mg, 49%) as a yellow solid: [α]²³_{D} +45 (c 0.07, THF); MALDIFT-HRMS m/z 421.0824 (M⁺, C₂₂H₁₆ClN₃O₄ requires 421.0824).

3-(6-Nitroindole-2-carbonyl)-1-(S)-(chloromethyl)-5-hydroxy-1,2-dihydro-3H-benz[e]indole (50A): (2.60 mg, 82%) as a yellow solid: [α]²³_{D} −26 (c 0.125, THF); ESI (positive) m/z 422 (M+H⁺, C₂₂H₁₇ClN₃O₄); ESI (negative) m/z 420 (M−H⁻, C₂₂H₁₅ClN₃O₄).

3-(7-Nitroindole-2-carbonyl)-1-(S)-(chloromethyl)-5-hydroxy-1,2-dihydro-3H-benz[e]indole (51A): (0.92 mg, 29%) as a yellow solid: [α]²³_{D} +9 (c 0.003, THF); ESI (positive) m/z 422 (M+H⁺, C₂₂H₁₇ClN₃O₄); ESI (negative) m/z 420 (M−H⁻, C₂₂H₁₅ClN₃O₄).

3-(5-Formylindole-2-carbonyl)-1-(S)-(chloromethyl)-5-hydroxy-1,2-dihydro-3H-benz[e]indole (52A): (1.18 mg, 39%) as a beige solid: [α]²³_{D} +9 (c 0.03, THF); MALDIFT-HRMS m/z 405.1012 (M+H⁺, C₂₃H₁₈ClN₂O₃ requires 405.1000).

3-(7-Formylindole-2-carbonyl)-1-(S)-(chloromethyl)-5-hydroxy-1,2-dihydro-3H-benz[e]indole (53A): (1.46 mg, 20%) as a yellow solid: [α]²³_{D} −33 (c 0.54, 2:1 CH₂CH₂/CDCl₃); MALDIFT-HRMS m/z 405.1013 (M+H⁺, C₂₃H₁₈ClN₂O₃ requires 405.1000).

3-(5-Acetylindole-2-carbonyl)-1-(S)-(chloromethyl)-5-hydroxy-1,2-dihydro-3H-benz[e]indole (54A): (2.06 mg, 66%) as a pale yellow solid: [α]²³_{D} +33 (c 0.1, THF); MALDIFT-HRMS m/z 418.1089 (M⁺, C₂₄H₁₉ClN₂O₃ requires 418.1084).

3-(7-Acetylindole-2-carbonyl)-1-(S)-(chloromethyl)-5-hydroxy-1,2-dihydro-3H-benz[e]indole (55A): (1.79 mg, 57%) as a pale yellow solid: [α]²³_{D} −1 (c 0.1, THF); MALDIFT-HRMS m/z 419.1157 (M+H⁺, C₂₄H₂₀ClN₂O₃ requires 419.1157).

3-(5-Propionylindole-2-carbonyl)-1-(S)-(chloromethyl)-5-hydroxy-1,2-dihydro-3H-benz[e]indole (56A): (2.05 mg, 63%) as a pale yellow solid: [α]²³_{D} +33 (c 0.1, THF); MALDIFT-HRMS m/z 432.1232 (M⁺, C₂₅H₂₁ClN₂O₃ requires 432.1235).

3-(5-Butyrylindole-2-carbonyl)-1-(S)-(chloromethyl)-5-hydroxy-1,2-dihydro-3H-benz[e]indole (57A): (2.15 mg, 64%) as a pale yellow solid: [α]²³_{D} +35 (c 0.1, THF); MALDIFT-HRMS m/z 446.1388 (M⁺, C₂₆H₂₃ClN₂O₃ requires 446.1392).

3-(5-Methoxycarbonylindole-2-carbonyl)-1-(S)-(chloromethyl)-5-hydroxy-1,2-dihydro-3H-benz[e]indole (58A): (1.64 mg, 50%) as a beige solid: [α]²³_{D} +25 (c 0.08, THF); MALDIFT-HRMS m/z 435.1096 (M+H⁺, C₂₄H₂₀ClN₂O₄ requires 435.1106).

3-(7-Methoxycarbonylindole-2-carbonyl)-1-(S)-(chloromethyl)-5-hydroxy-1,2-dihydro-3H-benz[e]indole (59A): (1.72 mg, 53%) as a yellow solid: [α]²³_{D} −6 (c 0.1, THF); MALDIFT-HRMS m/z 435.1118 (M+H⁺, C₂₄H₂₀ClN₂O₄ requires 435.1106).

3-(5-Ethoxycarbonylindole-2-carbonyl)-1-(S)-(chloromethyl)-5-hydroxy-1,2-dihydro-3H-benz[e]indole (60A): (1.64 mg, 50%) as a pale yellow solid: [α]²³_{D} +31 (c 0.1, THF); MALDIFT-HRMS m/z 449.1246 (M+H⁺, C₂₅H₂₂ClN₂O₄ requires 449.1263).

3-(5-Carbamoylindole-2-carbonyl)-1-(S)-(chloromethyl)-5-hydroxy-1,2-dihydro-3H-benz[e]indole (61A): (1.49 mg, 47%) as a beige solid: [α]²³_{D} +18 (c 0.08, THF); MALDIFT-HRMS m/z 419.1029 (M⁺, C₂₃H₁₈ClN₃O₃ requires 419.1031).

3-(5-Methylcarbamoylindole-2-carbonyl)-1-(S)-(chloromethyl)-5-hydroxy-1,2-dihydro-3H-benz[e]indole (62A): (0.97 mg, 30%) as a pale yellow solid: [α]²³_{D} +34 (c0.05, THF); MALDIFT-HRMS m/z 434.1276 (M+H⁺, C₂₄H₂₁ClN₃O₃ requires 434.1266).

3-(5-Dimethylcarbamoylindole-2-carbonyl)-1-(S)-(chloromethyl)-5-hydroxy-1,2-dihydro-3H-benz[e]indole (63A): (1.5 mg, 45%) as a pale yellow solid: [α]²³_{D} +25 (c 0.08, THF); MALDIFT-HRMS m/z 448.1422 (M+H⁺, C₂₅H₂₃ClN₃O₃ requires 448.1422).

3-(5-Methylsulfonylindole-2-carbonyl)-1-(S)-(chloromethyl)-5-hydroxy-1,2-dihydro-3H-benz[e]indole (64A): (1.29 mg, 38%) as a yellow solid: [α]²³_{D} +26 (c 0.05, THF); ESI (positive) m/z 455 (M+H⁺, C₂₃H₂₀ClN₂O₄S); ESI (negative) m/z 453 (M−H⁻, C₂₃H₁₈ClN₂O₄S).

3-(5-Ethylsulfonylindole-2-carbonyl)-1-(S)-(chloromethyl)-5-hydroxy-1,2-dihydro-3H-benz[e]indole (65A): (2.51 mg, 71%) as a pale yellow solid: [α]²³_{D} +30 (c 0.13, THF); MALDIFT-HRMS m/z 469.0966 (M+H⁺, C₂₄H₂₂ClN₂O₄S requires 469.0983).

3-(1,2-Dihydro-3H-pyrrolo[3,2-e]indole-7-carbonyl)-1-(S)-(chloromethyl)-5-hydroxy-1,2-dihydro-3H-benz[e]indole (66A): Prepared from 67A (6.3 mg, 12.1 μmol) by treatment with 4 N HCl/EtOAc (0.25 mL), followed by exposure to Et₃N (13.0 μL) in THF (1 mL) for 1 h which afforded 66A (5.0 mg, 100%) as a brown solid: [α]²³_{D} +34 (c 0.01, CH₂Cl₂); MALDIFT-HRMS m/z 417.1244 (M⁺, C₂₄H₂₀ClN₃O₂ requires 417.1244).

3-(N-t-Butyloxycarbonyl-1,2-dihydro-3H-pyrrolo[3,2-e]indole-7-carbonyl)-1-(S)-(chloromethyl)-5-hydroxy-1,2-dihydro-3H-benz[e]indole (67A): (6.3 mg, 67%) as a yellow solid: [α]²³_{D} −23 (c 0.04, CH₂Cl₂); ESI (positive) m/z 518 (M+H⁺, C₂₉H₂₉ClN₃O₄); ESI (negative) m/z 516 (M−H⁻, C₂₉H₂₇ClN₃O₄).

3-(Pyrrolo[3,2-e]indole-7-carbonyl)-1-(S)-(chloromethyl)-5-hydroxy-1,2-dihydro-3H-benz[e]indole (69A): (0.43 mg, 14%) as a pale yellow solid: [α]²³_{D} +184 (c 0.01, THF); MALDIFT-HRMS m/z 416.1156 (M+H⁺, C₂₄H₁₉ClN₃O₂ requires 416.1160).

3-(1,2-Dihydropyrrolo[3,2-e]benzofuran-7-carbonyl)-1-(S)-(chloromethyl)-5-hydroxy-1,2-dihydro-3H-benz[e]indole (70A): (0.8 mg, 53%) as a white solid: [α]²³_{D} +25 (c 0.2, acetone); MALDIFT-HRMS m/z 418.1094 (M⁺, C₁₁H₉NO₅ requires 418.1079).

3-(Pyrrolo[3,2-e]benzofuran-7-carbonyl)-1-(S)-(chloromethyl)-5-hydroxy-1,2-dihydro-3H-benz[e]indole (71A): (1.7 mg, 45%) as a white solid: [α]²³_{D} +42 (c 0.2, acetone); MALDIFT-HRMS m/z 416.0945 (M⁺, C₂₄H₁₇ClN₂O₃ requires 416.0928).

3-(6-Oxo-cyclopenten[e]indole-2-carbonyl)-1-(S)-(chloromethyl)-5-hydroxy-1,2-dihydro-3H-benz[e]indole (72A): (2.0 mg, 62%) as a pale yellow solid: [α]²³_{D} +75 (c 0.1, THF); MALDIFT-HRMS m/z 431.1157 (M+H⁺, C₂₅H₂₀ClN₂O₃ requires 431.1157).

3-(6-Oxo-cyclopenten[f]indole-2-carbonyl)-1-(S)-(chloromethyl)-5-hydroxy-1,2-dihydro-3H-benz[e]indole (73A): (1.5 mg, 46%) as a pale yellow solid: [α]²³_{D} +46 (c0.08, THF); MALDIFT-HRMS m/z 430.1063 (M⁺, C₂₅H₁₉ClN₂O₃ requires 430.1079).

3-([1,3]Dioxolo[e]indole-2-carbonyl)-1-(S)-(chloromethyl)-5-hydroxy-1,2-dihydro-3H-benz[e]indole (74A): (1.0 mg, 57%) as a yellow solid: [α]²³₀+30 (c 0.0065, CH₂Cl₂); MALDI-FTMS m/z 420.0869 (M⁺, C₂₃H₁₇ClN₂O₄ requires 420.0871).

3-([1,3]Dioxolo[f]indole-2-carbonyl)-1-(S)-(chloromethyl)-5-hydroxy-1,2-dihydro-3H-benz[e]indole (75A): (1.28 mg, 42%) as an off-white solid: [α]²³_{D} +1 (c 0.08, THF); MALDIFT-HRMS m/z 420.0888 (M⁺, C₂₃H₁₇ClN₂O₄ requires 420.0871). 76A: (3.8 mg, 62%) as a yellow solid: [α]²³_{D} −39 (c 0.02, CH₂Cl₂); MALDIFT-HRMS m/z 417.1371 (M+H⁺, C₂₅H₂₁ClN₂O₂ requires 417.1364).

3-[Benz[e]indole-2-carbonyl]-1-(chloromethyl)-5-hydroxy-1,2-dihydro-3H-benz[e]indole (77A): (1.60-2.30 mg, 37-54%) as a beige solid: (natural enantiomer [α]²³_{D} +100 (c 0.08, THF); unnatural enantiomer [α]²³₀-108 (c 0.1, THF); MALDI-HRMS m/z 426.1143 (M⁺, C₂₆H₁₉ClN₂O₂ requires 426.1135).

3-[Benz[f]indole-2-carbonyl]-1-(chloromethyl)-5-hydroxy-1,2-dihydro-3H-benz[e]indole (78A): (1.00-3.02 mg, 23-71%) as a yellow solid: (natural enantiomer) [α]²³₀+32 (c 0.05, THF); (unnatural enantiomer) [α]²³_{D} −40 (c 0.1, THF); MALDIFT-HRMS m/z 426.1149 (M⁺, C₂₆H₁₉ClN₂O₂ requires 426.1135).

Data for Spirocyclized Compounds:

N²-[(4-Methoxyindole-2-yl)carbonyl]-(8bR,9aS)-1,2,9,9a-tetrahydrocyclopropa[c]benz[e]indol-4-one (6B): A solution of 6A (0.98 mg, 2.4 μmol) and DBU (0.73 mg, 4.8 μmol) in 480 μL of CH₃CN/THF was stirred for 1 h at 0° C. The reaction mixture was allowed to reach 25° C. over 2 h, and then purified by preparative TLC (10×20 cm, CH₂Cl₂/acetone 1:1) to afford 6B (0.82 mg, 92%) as a white solid: [α]²³_{D} +90 (c 0.04, THF); MALDIFT-HRMS m/z 371.1398 (M+H⁺, C₂₃H₁₉N₂O₃ requires 371.1390).

N²-[(5-Methoxyindole-2-yl)carbonyl]-(8bR,9aS)-1,2,9,9a-tetrahydrocyclopropa[c]benz[e]indol-4-one (7B): A solution of 7A (2.28 mg, 5.6 μmol) and DBU (1.7 mg, 11.2 μmol) in 700 μL of CH₃CN was stirred for 1 h at 0° C. and 3 h at 25° C. The reaction mixture was then separated by preparative TLC (10×20 cm, CH₂Cl₂/acetone 1:1) to afford 7B (1.52 mg, 73%) as white solid: [α]²³_{D} +122 (c 0.08, THF); MALDIFT-HRMS m/z 371.1405 (M+H⁺, C₂₃H₁₉N₂O₃ requires 371.1390).

N²-[(6-Methoxyindole-2-yl)carbonyl]-(8bR,9aS)-1,2,9,9a-tetrahydrocyclopropa[c]benz[e]indol-4-one (8B): A solution of 8A (1.40 mg, 3.4 μmol) in 680 μL of CH₃CN/THF was treated with DBU (1.05 mg, 6.9 μmol) and then stirred for 1 h at 0° C. The reaction mixture was allowed to reach 25° C. over 1 h, and then purified by preparative TLC (10×20 cm, THF/hexane 3:2) to afford 8B (1.25 mg, 98%) as an off-white solid: [α]²³₀+147 (c 0.07, THF); MALDIFT-HRMS m/z 371.1392 (M+H⁺, C₂₃H₁₉N₂O₃ requires 371.1390).

N²-[(7-Methoxyindole-2-yl)carbonyl]-(8bR,9aS)-1,2,9,9a-tetrahydrocyclopropa[c]benz[e]indol-4-one (9B): A solution of 9A (1.30 mg, 3.2 μmol) and DBU (0.97 mg, 6.4 μmol) in 320 μL of CH₃CN and 320 μL of THF were stirred for 1.5 h at 0° C. and 1 h from 0 to 25° C. The reaction mixture was then purified by preparative TLC (10×20 cm, CH₂Cl₂/acetone 1:1) to afford 9B (1.11 mg, 94%) as a pale yellow solid: [α]²³₀+170 (c 0.05, THF); MALDIFT-HRMS m/z 371.1395 (M+H⁺, C₂₃H₁₉N₂O₃ requires 371.1390).

N²-[(4,5-Dimethoxyindole-2-yl)carbonyl]-(8bR,9aS)-1,2,9,9a-tetrahydrocyclopropa[c]benz[e]indol-4-one (10B): A solution of 10A (1.3 mg, 2.9 μmol) and DBU (2.7 mg, 17.8 μmol) in 500 μL of CH₃CN-THF was stirred for 1 h at 0° C. The reaction mixture was allowed to reach 25° C. over 1 h, and then purified by preparative TLC (10×20 cm, EtOAc/hexane 1:1) to afford 10B (0.42 mg, 35%) as a beige solid: [α]²³_{D} +225 (c=0.06, acetone); MALDIFT-HRMS m/z 401.1499 (M+H⁺, C₂₄H₂₀N₂O₄ requires 401.1496).

N²-[(5,6-Dimethoxyindole-2-yl)carbonyl]-(8bR,9aS)-1,2,9,9a-tetrahydrocyclopropa[c]benz[e]indol-4-one (11B): A solution of 11A (1.72 mg, 3.9 μmol) in 390 μL CH₃CN was treated with DBU (1.2 mg, 7.9 μmol) and stirred for 1 h at 0° C. The reaction mixture was allowed to reach 25° C. over 1 h, and then purified by preparative TLC (10×20 cm, THF/hexane 2:1) to afford 11B (1.29 mg, 82%) as a pale yellow solid: [α]²³_{D} +165 (c 0.07, THF); MS (ESI positive) m/z 399 (M+H⁺).

N²-[(6,7-Dimethoxyindole-2-yl)carbonyl]-(8bR,9aS)-1,2,9,9a-tetrahydrocyclopropa[c]benz[e]indol-4-one (12B): A solution of 12A (1.15 mg, 2.6 μmol) in 260 μL CH₃CN was treated with DBU (0.80 mg, 5.3 μmol) and then stirred for 2 h at 0° C. The reaction mixture was then purified by preparative TLC (10×20 cm, THF/hexane 1:1) to afford 12B (0.68 mg, 65%) as a white solid: [α]²³_{D} +168 (c 0.025, THF); MALDIFT-HRMS m/z 401.1509 (M+H⁺, C₂₄H₂₁N₂O₄ requires 401.1496).

N²-[(5,7-Dimethoxyindole-2-yl)carbonyl]-(8bR,9aS)-1,2,9,9a-tetrahydrocyclopropa[c]benz[e]indol-4-one (13B): A solution of 13A (1.0 mg, 2.3 μmol) in 230 μL CH₃CN was treated with DBU (0.70 mg, 4.6 μmol) and stirred for 1.5 h at 0° C. The reaction mixture was allowed to reach 25° C. over 2 h, and then purified by preparative TLC (10×20 cm, THF/hexane 1:1) to afford 13B (0.90 mg, 98%) as a pale yellow solid: [α]²³_{D} −58 (c 0.14, CHCl₃); MALDIFT-HRMS m/z 423.1317 (M+Na⁺, C₂₄H₂₀N₂NaO₄ requires 423.1315).

N²-[(5-Ethoxyindole-2-yl)carbonyl]-(8bR,9aS)-1,2,9,9a-tetrahydrocyclopropa[c]benz[e]indol-4-one (15B): A solution of 15A (1.56 mg, 3.7 μmol) and DBU (1.13 mg, 7.4 μmol) in 370 μL of CH₃CN and 370 μL of THF was stirred for 1 h at 0° C. The reaction mixture was then purified by preparative TLC (10×20 cm, CH₂Cl₂/acetone 1:1) to afford 15B (1.35 mg, 94%) as a pale yellow solid: [α]²³_{D} +144 (c 0.1, THF); MALDIFT-HRMS m/z 384.1483 (M⁺, C₂₄H₂₁N₂O₃ requires 384.1474).

N²-[(5-Propyloxyindole-2-yl)carbonyl]-(8bR,9aS)-1,2,9,9a-tetrahydrocyclopropa[c]benz[e]indol-4-one (16B): A solution of 16A (1.80 mg, 4.1 μmol) and DBU (1.26 mg, 8.2 μmol) in 410 μL of CH₃CN and 205 μL of THF was stirred for 1.5 h at 0° C. The reaction mixture was then purified by preparative TLC (10×20 cm, CH₂Cl₂/acetone 1:1) to afford 16B (1.15 mg, 70%) as a pale yellow solid: [α]²³_{D} +139 (c 0.06, THF); MALDIFT-HRMS m/z 384.1483 (M⁺, C₂₅H₂₁N₂O₃ requires 384.1474).

N²-[(5-Butyloxyindole-2-yl)carbonyl]-(8bR,9aS)-1,2,9,9a-tetrahydrocyclopropa[c]benz[e]indol-4-one (17B): A solution of 17A (1.26 mg, 2.8 μmol) and DBU (0.86 mg, 5.6 μmol) in 330 μL of CH₃CN and 100 μL of THF was stirred for 1.5 h at 0° C. The reaction mixture was then purified by preparative TLC (10×20 cm, CH₂Cl₂/acetone 1:1) to afford 17B (1.02 mg, 88%) as a beige solid: [α]²³_{D} +124 (c 0.05, THF); MALDIFT-HRMS m/z 413.1855 (M+H⁺, C₂₆H₂₄N₂O₃ requires 413.1860).

N²-[(5-Benzyloxyindole-2-yl)carbonyl]-(8bR,9aS)-1,2,9,9a-tetrahydrocyclopropa[c]benz[e]indol-4-one (18B): A solution of 18A (0.88 mg, 1.8 μmol) and DBU (0.55 mg, 3.6 μmol) in 180 μL of CH₃CN and 180 μL of THF was stirred for 2.5 h at 0° C. The reaction mixture was then purified by preparative TLC (10×20 cm, CH₂Cl₂/acetone 1:1) to afford 18B (0.74 mg, 91%) as pale yellow solid: [α]²³₀+92 (c 0.1, THF); MALDIFT-HRMS m/z 447.1693 (M+H⁺, C₂₉H₂₂N₂O₃ requires 447.1703).

N²-[(5-Thiomethylindole-2-yl)carbonyl]-(8bR,9aS)-1,2,9,9a-tetrahydrocyclopropa[c]benz-[e]indol-4-one (21B): A solution of 21A (1.0 mg, 2.4 μmol) and DBU (0.72 mg, 4.7 μmol) in 240 μL of CH₃CN was stirred for 1 h at 0° C. The reaction mixture was allowed to reach 25° C. over 2 h, and then purified by preparative TLC (10×20 cm, THF/hexane 2:1) to afford 21B (0.51 mg, 56%) as a beige solid: [α]²³_{D} +136 (c 0.025, THF); MALDIFT-HRMS m/z 387.1164 (M+H⁺, C₂₃H₁₉N₂O₂S requires 387.1162).

N²-[(7-Thiomethylindole-2-yl)carbonyl]-(8bR,9aS)-1,2,9,9a-tetrahydrocyclopropa[c]benz[e]indol-4-one (22B): A solution of 22A (1.5 mg, 3.6 μmol) and DBU (1.08 mg, 7.1 μmol) in 360 μL of CH₃CN was stirred for 1 h at 0° C. The reaction mixture was allowed to reach 25° C. over 1 h, and then purified by preparative TLC (10×20 cm, THF/hexane 1:1) to afford 22B (1.01 mg, 74%) as beige solid: [α]²³_{D} +184 (c 0.05, THF); MALDIFT-HRMS m/z 387.1176 (M+H⁺, C₂₃H₁₉N₂O₂S requires 387.1162).

N²-[(5-Thioethylindole-2-yl)carbonyl]-(8bR,9aS)-1,2,9,9a-tetrahydrocyclopropa[c]benz[e]indol-4-one (23B): A solution of 23A (1.7 mg, 3.9 μmol) and DBU (1.18 mg, 7.8 μmol) in 390 μL of CH₃CN was stirred for 1.5 h at 0° C. The reaction mixture was allowed to reach 25° C. over 1.5 h, and then purified by preparative TLC (10×20 cm, THF/hexane 1:1) to afford 23B (1.28 mg, 82%) as pale yellow solid: [α]²³_{D} +135 (c 0.07, THF); MALDIFT-HRMS m/z 423.1131 (M+Na⁺, C₂₄H₂₀N₂NaO₂S requires 423.1138).

N²-[(Indole-2-yl)carbonyl]-(8bR,9aS)-1,2,9,9a-tetrahydrocyclopropa[c]benz[e]indol-4-one (5B, CBI-indole): A solution of 5A (1.50 mg, 4 μmol) and DBU (1.2 mg, 8 μmol) in 400 μL of CH₃CN was stirred for 1 h at 0° C. and the reaction mixture was purified by preparative TLC (10×20 cm, CH₂Cl₂/acetone 1:1) to afford 5B (1.25 mg, 93%) as a white solid: [α]²³_{D} +159 (c 0.07, THF); MALDIFT-HRMS m/z 341.1276 (M+H⁺, C₂₂H₁₇N₂O₂ requires 341.1284).

N²-[(5-Methylindole-2-yl)carbonyl]-(8bR,9aS)-1,2,9,9a-tetrahydrocyclopropa[c]benz[e]in-dol-4-one (24B): A solution of 24A (1.52 mg, 3.9 μmol) and DBU (1.18 mg, 7.8 μmol) in 390 μL of CH₃CN was stirred for 1.5 h at 0° C. and the reaction mixture was purified by preparative TLC (10×20 cm, CH₂Cl₂/acetone 1:1) to afford 24B (1.20 mg, 87%) as a white solid: [α]²³_{D} +149 (c 0.07, THF); MALDIFT-HRMS m/z 355.1441 (M+H⁺, C₂₃H₁₉N₂O₂ requires 355.1441).

N²-[(5-Ethylindole-2-yl)carbonyl]-(8bR,9aS)-1,2,9,9a-tetrahydrocyclopropa[c]benz[e]indol-4-one (25B): A solution of 25A (1.1 mg, 2.7 μmol) and DBU (1.18 mg, 7.8 μmol) in 270 μL of CH₃CN was stirred for 1 h at 0° C. and 1 h at 25° C. The reaction mixture was purified by preparative TLC (10×20 cm, THF/hexane 1:1) to afford 25B (0.61 mg, 61%) as a white solid: [α]²³_{D} +188 (c 0.025, THF); MALDIFT-HRMS m/z 369.1585 (M+H⁺, C₂₄H₂₀N₂O₂ requires 369.1597).

N²-[(5-Methoxymethylindole-2-yl)carbonyl]-(8bR,9aS)-1,2,9,9a-tetrahydrocyclopropa[c]benz[e]indol-4-one (26B): A solution of 26A (1.4 mg, 3.3 μmol) and DBU (1.01 mg, 6.6 μmol) in 330 μL of CH₃CN was stirred for 1 h at 0° C. The reaction mixture was then purified by preparative TLC (10×20 cm, THF/hexane 3:2) to afford 26B (1.09 mg, 85%) as a beige solid: [α]²³_{D} +128 (c 0.05, THF); MALDIFT-HRMS m/z 385.1558 (M+H⁺, C₂₄H₂₁N₂O₃ requires 385.1547).

N²-[(7-Methoxymethylindole-2-yl)carbonyl]-(8bR,9aS)-1,2,9,9a-tetrahydrocyclopropa[c]-benz[e]indol-4-one (27B): A solution of 27A (1.15 mg, 2.7 μmol) and DBU (0.83 mg, 5.5 μmol) in 270 μL of CH₃CN was stirred for 1 h at 0° C., and then purified by preparative TLC (10×20 cm, THF/hexane 3:2) to afford 27B (0.91 mg, 87%) as a white solid: [α]²³_{D} +125 (c 0.04, THF); MALDIFT-HRMS m/z 385.1547 (M+H⁺, C₂₄H₂₁N₂O₂ requires 385.1547).

N²-[(5-Bromoindole-2-yl)carbonyl]-(8bR,9aS)-1,2,9,9a-tetrahydrocyclopropa[c]benz[e]in-dol-4-one (28B): A solution of 28A (1.72 mg, 3.8 μmol) in 860 μL of THF was treated with NaH (0.18 mg, 7.6 μmol) and was stirred for 2 h at 0° C., then warmed to 25° C. over 1 h and stirred for 3 h. The reaction mixture was purified by preparative TLC (10×20 cm, CH₂Cl₂/acetone 1:1) to afford 28B (0.60 mg, 38%) as a beige solid: [α]²³_{D} +66 (c 0.03, THF); MALDIFT-HRMS m/z 419.0408 (M+H⁺, C₂₂H₁₆BrN₂O₂ requires 419.0390).

N²-[(5-Chloroindole-2-yl)carbonyl]-(8bR,9aS)-1,2,9,9a-tetrahydrocyclopropa[c]benz[e]in-dol-4-one (29B): A solution of 29A (1.53 mg, 3.8 μmol) in 380 μL of DMF was treated with NaH (0.18 mg, 7.6 μmol) and was stirred for 1.5 h at 0° C. The reaction mixture was then purified by preparative TLC (10×20 cm, CH₂Cl₂/acetone 1:1) to afford 29B (1.08 mg, 77%) as a beige solid: [α]²³_{D} +108 (c 0.05, THF); MALDIFT-HRMS m/z 375.0901 (M+H⁺, C₂₂H₁₆ClN₂O₂ requires 375.0895).

N²-[(5-Azidoindole-2-yl)carbonyl]-(8bR,9aS)-1,2,9,9a-tetrahydrocyclopropa[c]benz[e]indol-4-one (30B): A solution of 30A (1.07 mg, 2.6 μmol) and DBU (0.78 mg, 5.1 μmol) in 260 μL of CH₃CN was stirred for 1 h at 0° C. The reaction mixture was then purified by preparative TLC (10×20 cm, CH₂Cl₂/acetone 1:1) to afford 30B (0.82 mg, 84%) as a beige solid: [α]²³_{D} +100 (c 0.05, THF); MS (ESI positive) m/z 382 (M+H⁺); MS (ESI negative) m/z 380 (M−H—).

N²-[(5-Cyanoindole-2-yl)carbonyl]-(8bR,9aS)-1,2,9,9a-tetrahydrocyclopropa[c]benz[e]in-dol-4-one (31B): A solution of 31A (2.10 mg, 5.2 μmol) in 520 μL of DMF was treated with NaH (0.25 mg, 10.5 μmol) and was stirred for 1.5 h at 0° C. The reaction mixture was then purified by preparative TLC (10×20 cm, CH₂Cl₂/acetone 1:1) to afford 31B (0.41 mg, 21%) as a beige solid: [α]²³_{D} +80 (c 0.025, THF); MALDIFT-HRMS m/z 366.1235 (M+H⁺, C₂₃H₁₆N₃O₂ requires 366.1237).

N²-[(7-Cyanoindole-2-yl)carbonyl]-(8bR,9aS)-1,2,9,9a-tetrahydrocyclopropa[c]benz[e]in-dol-4-one (32B): A solution of 32A (0.69 mg, 1.7 mmol) in 170 μL of DMF was treated with NaH (0.08 mg, 3.4 μmol) and was stirred for 1.5 h at 0° C. The reaction mixture was purified by preparative TLC (10×20 cm, CH₂Cl₂/acetone 1:1) to afford 32B (0.39 mg, 62%) as a pale yellow solid: [α]²³_{D} +124 (c 0.05, THF); MALDIFT-HRMS m/z 365.1169 (M⁺, C₂₃H₁₅N₃O₂ requires 365.1164).

N²-[(5-Vinylindole-2-yl)carbonyl]-(8bR,9aS)-1,2,9,9a-tetrahydrocyclopropa[c]benz[e]indol-4-one (33B): A solution of 33A (1.35 mg, 3.4 μmol) and DBU (1.02 mg, 6.7 μmol) in 600 μL of CH₃CN/THF was stirred for 1 h at 0° C. and 1 h at 25° C. The reaction mixture was then purified by preparative TLC (10×20 cm, THF/hexane 1:1) to afford 33B (0.80 mg, 65%) as a beige solid: [α]²³_{D} +159 (c 0.03, THF); MALDIFT-HRMS m/z 367.1442 (M+H⁺, C₂₄H₁₉N₂O₂ requires 367.1441).

N²-[(7-Vinylindole-2-yl)carbonyl]-(8bR,9aS)-1,2,9,9a-tetrahydrocyclopropa[c]benz[e]indol-4-one (34B): A solution of 34A (0.95 mg, 2.4 μmol) and DBU (0.72 mg, 4.7 μmol) in 400 μL of CH₃CN/THF was stirred for 1 h at 0° C. and 1 h at 25° C. The reaction mixture was purified by preparative TLC (10×20 cm, THF/hexane 1:1) to afford 34B (0.76 mg, 88%) as a beige solid: [α]²³_{D} +198 (c 0.03, THF); MALDIFT-HRMS m/z 367.1440 (M+H⁺, C₂₄H₁₉N₂O₂ requires 367.1441).

N²-[(5-Isopropenylindole-2-yl)carbonyl]-(8bR,9aS)-1,2,9,9a-tetrahydrocyclopropa[c]-benz[e]indol-4-one (35B): A solution of 35A (1.3 mg, 3.1 μmol) and DBU (0.95 mg, 6.2 μmol) in 310 μL of CH₃CN was stirred for 1 h at 0° C. and warmed over 1.5 h to 25° C. The reaction mixture was purified by preparative TLC (10×20 cm, THF/hexane 1:1) to afford 35B (0.79 mg, 66%) as an off-white solid: [α]²³_{D} +116 (c 0.05, THF); MALDIFT-HRMS m/z 381.1591 (M+H⁺, C₂₅H₂₁N₂O₂ requires 381.1597).

N²-[(5-Ethynylindole-2-yl)carbonyl]-(8bR,9aS)-1,2,9,9a-tetrahydrocyclopropa[c]benz[e]in-dol-4-one (37B): A solution of 37A (1.52 mg, 3.9 μmol) and DBU (1.18 mg, 7.8 μmol) in 390 μL of CH₃CN was stirred for 1.5 h at 0° C. The reaction mixture was then purified by preparative TLC (10×20 cm, CH₂Cl₂/acetone 1:1) to afford 37B (1.20 mg, 87%) as a white solid: [α]²³_{D} +149 (c 0.07, THF); MALDIFT-HRMS m/z 365.1278 (M+H⁺, C₂₄H₁₇N₂O₂ requires 365.1284).

N²-{5-[1-(Propynyl)indole-2-yl]carbonyl}-(8bR,9aS)-1,2,9,9a-tetrahydrocyclopropa[c]-benz[e]indol-4-one (38B): A solution of 38A (1.00 mg, 2.4 μmol) and DBU (0.73 mg, 4.8 μmol) in 240 μL of CH₃CN was stirred for 1 h at 0° C. The reaction mixture was then purified by preparative TLC (10×20 cm, CH₂Cl₂/acetone 1:1) to afford 38B (0.72 mg, 79%) as a beige solid: [α]²³_{D} +106 (c 0.07, THF); MALDIFT-HRMS m/z 378.1362 (M⁺, C₂₅H₁₈N₂O₂ requires 378.1368).

N²-[(5-Aminoindole-2-yl)-carbonyl]-(8bR,9aS)-1,2,9,9a-tetrahydrocyclopropa[c]benz[e]in-dol-4-one (40B): A sample of 43A (0.75 mg, 1.5 μmol) was treated with 4 M HCl in EtOAc for 1 h at 25° C. and then the solvent was evaporated. Crude 40A was dissolved in 300 μL of CH₃CN/THF and treated with DBU (0.69 mg, 2 μmol) for 1 h at 0° C. and then allowed to reach 25° C. over 30 min. The reaction mixture was purified by preparative TLC (10×20 cm, THF) to afford 40B (0.46 mg, 85%) as a yellow solid: [α]²³_{D} +92 (c 0.025, THF); MALDIFT-HRMS m/z 356.1398 (M+H⁺, C₂₂H₁₈N₃O₂ requires 356.1393).

N²-[(5-Acetylaminoindole-2-yl)carbonyl]-(8bR,9aS)-1,2,9,9a-tetrahydrocyclopropa[c]-benz[e]indol-4-one (44B): A solution of 44A (1.37 mg, 3.2 μmol) and DBU (0.96 mg, 6.3 μmol) in 320 μL of CH₃CN and 320 μL of THF was stirred for 1 h at 0° C. and 2 h from 0 to 25° C. The reaction mixture was then purified by preparative TLC (10×20 cm, CH₂Cl₂/acetone 1:1) to afford 44B (0.95 mg, 76%) as pale yellow solid: [α]²³_{D} +90 (c 0.1, THF); MALDIFT-HRMS m/z 398.1512 (M+H⁺, C₂₄H₂₀N₃O₃ requires 398.1499).

N²-[(5-Propionylaminoindole-2-yl)carbonyl]-(8bR,9aS)-1,2,9,9a-tetrahydrocyclopropa[c]-benz[e]indol-4-one (45B): A solution of 45A (0.89 mg, 2 μmol) and DBU (0.60 mg, 4 μmol) in 200 μL of CH₃CN and 200 μL of THF was stirred for 1 h at 0° C. and 2 h from 0 to 25° C. The reaction mixture was purified by preparative TLC (10×20 cm, CH₂Cl₂/acetone 1:1) to afford 45B (0.56 mg, 68%) as pale yellow solid: [α]²³_{D} +56 (c 0.05, THF); MALDIFT-HRMS m/z 412.1658 (M+H⁺, C₂₅H₂₂N₃O₃ requires 412.1656).

N²-[(5-Butyrylaminoindole-2-yl)carbonyl]-(8bR,9aS)-1,2,9,9a-tetrahydrocyclopropa[c]benz[e]indol-4-one (46B): A solution of 46A (1.22 mg, 2.6 μmol) and DBU (0.80 mg, 5.3 μmol) in 260 μL of CH₃CN and 260 μL of THF was stirred for 1.5 h at 0° C. and 2 h from 0 to 25° C. The reaction mixture was then purified by preparative TLC (10×20 cm, CH₂Cl₂/acetone 1:1) to afford 46B (1.10 mg, 99%) as pale yellow solid: [α]²³_{D} +92 (c 0.1, THF); MALDIFT-HRMS m/z 426.1819 (M+H⁺, C₂₆H₂₃N₃O₃ requires 426.1812).

N²-{[5-(N-Acetyl-N-methyl)aminoindole-2-yl]carbonyl}-(8bR,9aS)-1,2,9,9a-tetrahydrocyclopropa[c]benz[e]indol-4-one (47B): A solution of 47A (1.20 mg, 2.7 μmol) and DBU (0.82 mg, 5.4 μmol) in 270 μL of CH₃CN and 270 μL of THF was stirred for 2 h at 0° C. and 1 h from 0 to 25° C. The reaction mixture was then purified by preparative TLC (10×20 cm, CH₂Cl₂/acetone 1:1) to afford 47B (1.05 mg, 95%) as pale yellow solid: [α]²³_{D} +80 (c 0.1, THF); MALDIFT-HRMS m/z 412.1664 (M+H⁺, C₂₅H₂₂N₃O₃ requires 412.1656).

N²-[(7-Formylindole-2-yl)carbonyl]-(8bR,9aS)-1,2,9,9a-tetrahydrocyclopropa[c]benz[e]in-dol-4-one (53B): A solution of 53A (940 μg, 2.3 μmol) and DBU (1.9 μL, 12.3 μmol) in 50 μL of CH₃CN was stirred for 2 h at 23° C. and then purified by preparative TLC (10×20 cm, EtOAc) to afford 53B (0.41 mg, 48%) as a yellow solid: [α]²³_{D} +226 (c 0.0023, CHCl₃); MALDIFT-HRMS m/z 369.1242 (M+H⁺, C₂₃H₁₆N₂O₃ requires 369.1234).

N²-[(5-Acetylindole-2-yl)carbonyl]-(8bR,9aS)-1,2,9,9a-tetrahydrocyclopropa[c]benz[e]in-dol-4-one (54B): A solution of 54A (1.6 mg, 3.8 μmol) in 380 μL of DMF was treated with NaH (0.18 mg, 7.6 μmol) and was stirred for 1.5 h at 0° C. The reaction mixture was then purified by preparative TLC (10×20 cm, CH₂Cl₂/acetone 1:1) to afford 54B (1.08 mg, 77%) as a pale yellow solid: [α]²³_{D} +48 (c 0.05, THF); MALDIFT-HRMS m/z 383.1391 (M+H⁺, C₂₄H₁₉N₂O₃ requires 383.1390).

N²-[(7-Acetylindole-2-yl)carbonyl]-(8bR,9aS)-1,2,9,9a-tetrahydrocyclopropa[c]benz[e]in-dol-4-one (55B): A solution of 55A (1.55 mg, 3.7 μmol) in 370 μL of DMF was treated with NaH (0.18 mg, 7.4 μmol) and was stirred for 1 h at 0° C. The reaction mixture was purified by preparative TLC (10×20 cm, CH₂Cl₂/acetone 1:1) to afford 55B (1.08 mg, 77%) as a white solid: [α]²³_{D} +196 (c 0.1, THF); MALDIFT-HRMS m/z 383.1392 (M+H⁺, C₂₄H₁₁N₂O₃ requires 383.1390).

N²-[(5-Propionylindole-2-yl)carbonyl]-(8bR,9aS)-1,2,9,9a-tetrahydrocyclopropa[c]benz[e]-indol-4-one (56B): A solution of 56A (1.95 mg, 4.5 μmol) in 450 μL of DMF was treated with NaH (0.22 mg, 9.0 mmol) and was stirred for 1.5 h at 0° C. The reaction mixture was purified by preparative TLC (10×20 cm, CH₂Cl₂/acetone 1:1) to afford 56B (0.86 mg, 46%) as a pale yellow solid: [α]²³₀+120 (c 0.1, THF); MALDIFT-HRMS m/z 397.1546 (M+H⁺, C₂₅H₂₁N₂O₃ requires 397.1547).

N²-[(5-Butyrylindole-2-yl)carbonyl]-(8bR,9aS)-1,2,9,9a-tetrahydrocyclopropa[c]benz[e]indol-4-one (57B): A solution of 57A (1.92 mg, 4.3 μmol) in 430 μL of DMF was treated with NaH (0.21 mg, 8.6 μmol) and was stirred for 1.5 h at 0° C. The reaction mixture was then purified by preparative TLC (10×20 cm, CH₂Cl₂/acetone 1:1) to afford 57B (0.55 mg, 31%) as a pale yellow solid: [α]²³_{D} +96 (c 0.1, THF); MALDIFT-HRMS m/z 411.1708 (M+H⁺, C₂₆H₂₂N₂O₃ requires 411.1703).

N²-[(1,2-Dihydropyrrolo[3,2-e]benzofuran-7-yl)carbonyl]-(8bR,9aS)-1,2,9,9a-tetrahydro-cyclopropa[c]benz[e]indol-4-one (70B): A solution of 70A (0.8 mg, 1.9 μmol) and DBU (1.7 mg, 11.5 μmol) in 500 μL of CH₃CN-THF was stirred for 1 h at 0° C. The reaction mixture was allowed to reach 25° C. over 1 h, and then purified by preparative TLC (10×20 cm, EtOAc/hexane 1:1) to afford 70B (0.5 mg, 69%) as beige solid: [α]²³_{D} +90 (c=0.07, acetone); MALDIFT-HRMS m/z 383.1417 (M+H⁺, C₂₄H₁₈N₂O₃ requires 383.1396).

N²-[(Pyrrolo[3,2-e]benzofuran-7-yl)carbonyl]-(8 bR,9aS)-1,2,9,9a-tetrahydrocyclopropa[c]-benz[e]indol-4-one (71B): A solution of 71A (0.6 mg, 1.4 μmol) and DBU (1.3 mg, 8.6 μmol) in 500 μL of CH₃CN-THF was stirred for 1 h at 0° C. The reaction mixture was allowed to reach 25° C. over 1 h, and then purified by preparative TLC (10×20 cm, EtOAc/hexane 1:1) to afford 71B (0.30 mg, 55%) as a beige solid: [α]²³_{D} +185 (c=0.04, acetone); MALDIFT-HRMS m/z 381.1231 (M+H⁺, C₂₄H₁₆N₂O₃ requires 381.1234).

N²-[([1,3]Dioxolo[f]indole-2-yl)carbonyl]-(8bR,9aS)-1,2,9,9a-tetrahydrocyclopropa[c]-benz[e]indol-4-one (75B): A solution of 75A (1.25 mg, 3.1 μmol) in 310 μL of CH₃CN was treated with DBU (0.93 mg, 6.1 μmol) and then stirred for 1 h at 0° C. The reaction mixture was allowed to reach 25° C. over 1 h, and then purified by preparative TLC (10×20 cm, THF/hexane 2:1) to afford 75B (0.90 mg, 77%) as a pale yellow solid: [α]²³_{D} +153 (c 0.04, THF); MALDIFT-HRMS m/z 385.1196 (M+H⁺, C₂₃H₁₇N₂O₄ requires 385.1183).

76B: A solution of 76A (700 μg, 1.7 μmol) and DBU (1.4 mg, 8.9 μmol) in 60 μL of CH₃CN was stirred for 2 h at 23° C. and then purified by preparative TLC (10×20 cm, THF/hexane 1:1) to afford 76B (590 μg, 92%) as a yellow solid: [α]²³_{D} +26 (c 0.004, CH₂Cl₂); MALDIFT-HRMS m/z 381.1599 (M+H⁺, C₂₅H₂₀N₂O₂ requires 381.1597).

N²-[(Benz[e]indole-2-yl)carbonyl]-1,2,9,9a-tetrahydrocyclopropa[c]benz[e]indol-4-one (77B):

A solution of 77A (2.05 mg, 4.8 μmol) and NaH (0.23 mg, 9.6 μmol) in 480 μL of THF was stirred for 1 h at 0° C. and the reaction mixture was separated by preparative TLC (10×20 cm, THF) and (10×20 cm, CH₂Cl₂/acetone 3:1) to afford 77B (1.50 mg, 80%) as a beige solid: (natural enantiomer) [α]²³₀+114 (c 0.07, THF); (unnatural enantiomer) [α]²³_{D} −111 (c 0.06, THF); MALDIFT-HRMS m/z 391.1454 (M+H⁺, C₂₆H₁₁N₂O₂ requires 391.1441).

N²-[(Benz[f]indole-2-yl)carbonyl]-1,2,9,9a-tetrahydrocyclopropa[c]benz[e]indol-4-one (78B):

A solution of 78A (1.00 mg, 2.3 μmol) and DBU (0.71 mg, 4.7 μmol) in 240 μL of CH₃CN was stirred for 1 h at 0° C. and the reaction mixture was separated by two consecutive preparative TLC (10×20 cm, CH₂Cl₂/acetone 1:1) to afford 78B (0.53 mg, 58%) as a yellow solid: (natural enantiomer) [α]²³₀+126 (c 0.03, THF); (unnatural enantiomer) [α]²³_{D} −136 (c 0.025, THF); MALDIFT-HRMS m/z 391.1430 (M+H⁺, C₂₆H₁₉N₂O₂ requires 391.1441).

5,6- or 6,7-Dimethoxyindole-2-carboxylic acid (104, 105). 3,4-Dimethoxybenzaldehyde 101 (332 mg, 2 mmol) and methyl azidoacetate (691 mg, 6 mmol) in MeOH (4 mL) were treated at −20° C. with a solution of sodium methoxide generated in situ with Na (138 mg, 6 mmol) and 4 mL of MeOH. The reaction mixture was allowed to warm over 1 h at 25° C. and stirred for 2 h at this temperature, then quenched with the addition of ice water (20 mL). The yellow precipitate was filtered, washed with H₂O (15 mL) and dried by azeotropic distillation with benzene to afford methyl 2-azido-3-(3,4-dimethoxyphenyl)acrylate (215 mg, 41%) as a yellow solid: ¹H NMR (CDCl₃, 500 MHz) δ 7.49 (1H, d, J=1.8 Hz), 7.31 (1H, dd, J=8.5, 1.8 Hz), 6.83-6.85 (2H, m), 3.90 (3H, s), 3.89 (3H, s), 3.87 (3H, s).

Methyl 2-azido-3-(3,4-dimethoxyphenyl)acrylate (158 mg, 0.6 mmol) in m-xylene (4 mL) was added over 10 min to refluxing m-xylene (10 mL). The reaction mixture was stirred for 15 min at 140° C., then concentrated in vacuo. Flash chromatography (2×15 cm, SiO₂, 17% to 33% EtOAc/hexane) afforded methyl 5,6-dimethoxyindole-2-carboxylate 102 (110 mg, 78%) as a pale yellow solid: mp 162° C.; IR (film) vₘₐₓ 3324, 2935, 1702, 1513, 1260, 1203, 1099 cm⁻¹.

102 (47.1 mg, 0.2 mmol) in dioxane/H₂O (4:1, 1 mL) was treated with 4 M LiOH (200 μL) for 12 h at 25° C., then quenched with the addition of 1 M HCl (2 mL) and ice, and extracted with EtOAc (2×20 mL). The combined organic layers were dried (Na₂SO₄), and concentrated in vacuo to afford 104 (44 mg, quant.) as a gray solid: mp 202° C. (dec); ¹H NMR (DMSO-d₆, 500 MHz) δ 12.57 (1H, br s), 11.45 (1H, s), 7.07 (1H, s), 6.95 (1H, d, J=2.2 Hz), 6.86 (1H, s), 3.78 (3H, s), 3.75 (3H, s); ¹³C NMR (DMSO-d₆, 125 MHz) δ 162.6, 149.1, 145.5, 132.4, 126.4, 119.7, 107.5, 102.5, 94.3, 55.6, 55.4.

103 (13 mg, 0.055 mmol) in dioxane/H₂O (4:1, 300 μL) was treated with 4 M LiOH (26 μL) for 12 h at 25° C., then quenched with the addition of 1 M HCl (1 mL) and ice, and extracted with EtOAc (2×20 mL). The combined organic layers were dried (Na₂SO₄), and concentrated in vacuo to afford 105 (12 mg, 98%) as a beige solid: mp 133° C. (lit. (De Antoni, A.; et al. Gazz. Chim. Ital. 1970, 100, 1056-1060) mp 158° C.); IR (film) vₘₐₓ 3292, 2936, 1694, 1514, 1255, 1099 cm⁻¹.

5,7-Dimethoxyindole-2-carboxylic acid (108): 3,5-dimethoxybenzaldehyde 106 (665 mg, 4 mmol) and methyl azidoacetate (1.38 g, 12 mmol) in 8 ml MeOH were treated at −5° C. with 8 mL of sodium methoxide generated in situ with Na (276 mg, 12 mmol) and 8 mL of MeOH. The reaction mixture was stirred for 1 h at −5° C. and then allowed to warm over 2 h to 25° C., then quenched with the addition of ice water (20 mL). The yellow precipitate was filtered, washed with H₂O (15 mL) and dried by azeotropic distillation with benzene to afford methyl 2-azido-3-(3,5-dimethoxyphenyl)acrylate (603 mg, 57%) as a yellow solid: ¹H NMR (CDCl₃, 500 MHz) δ 6.99 (2H, d, J=2.2 Hz), 6.84 (1H, s), 6.47 (1H, t, J=2.0 Hz), 3.91 (3H, s), 3.82 (6H, s).

Methyl 2-azido-3-(3,5-dimethoxyphenyl)acrylate (527 mg, 2 mmol) in m-xylene (20 mL) was stirred for 3 h at 135° C., then concentrated in vacuo. Flash chromatography (1.5×20 cm, SiO₂, 6% to 9% EtOAc/hexane) afforded methyl 5,7-dimethoxyindole-2-carboxylate 107 (355 mg, 75%) as a pale yellow solid: ¹H NMR (acetone-d₆, 500 MHz) δ 10.66 (1H, br s), 7.07 (1H, d, J=2.1 Hz), 6.69 (1H, d, J=2.1 Hz), 6.45 (1H, d, J=2.1 Hz), 3.92 (3H, s), 3.86 (3H, s), 3.79 (3H, s); ¹³C NMR (acetone-d₆, 125 MHz) δ 162.5, 156.6, 148.2, 129.1, 128.4, 125.1, 109.1, 98.1, 94.5, 55.9, 55.8.

107 (70.6 mg, 0.3 mmol) in dioxane/H₂O (4:1, 1.5 mL) was treated with 4 M LiOH (300 μL) for 18 h at 25° C., then quenched with the addition of 1 M HCl (3 mL) and ice, and extracted with EtOAc (2×20 mL). The combined organic layers were dried (Na₂SO₄), and concentrated in vacuo to afford 108 (62 mg, 94%) as a white solid: mp 205° C. (dec) (lit. 214-215° C.); ¹H NMR (DMSO-d₆, 500 MHz) δ 12.71 (1H, brs), 11.52 (1H, s), 6.98 (1H, d, J=2.0 Hz), 6.65 (1H, d, J=2.0 Hz), 6.42 (1H, d, J=2.0 Hz), 3.87 (3H, s), 3.74 (3H, s).

5-Ethoxyindole-2-carboxylic acid (112): A solution of ethyl 5-benzyloxyindole-2-carboxylate 109 (295 mg, 1.0 mmol), Boc₂O (436 mg, 2.0 mmol) and DMAP (122 mg, 1.0 mmol) in 8 mL of CH₂Cl₂ and 2 mL of THF was stirred for 1 h at 25° C., then quenched with the addition of 0.2 M HCl saturated with NaCl (20 mL). The reaction mixture was extracted with CH₂Cl₂ (3×25 mL). The combined organic layers were dried (Na₂SO₄), and concentrated in vacuo to obtain crude product (385 mg, 97%) as an oil: IR (film) vₘₐₓ 2981, 2934, 1732, 1452, 1393, 1372, 1324, 1296, 1278, 1260, 1210, 1161, 1123, 1070 cm⁻¹; MALDIFT-HRMS m/z 418.1611 (M+Na⁺, C₂₃H₂₅NNaO₂ requires 418.1625).

A solution of ethyl 5-benzyloxy-N-(tert-butyloxycarbonyl)indole-2-carboxylate (305 mg, 0.77 mmol), Pd(OAc)₂ (7.8 mg, 0.035 mmol), Et₃N (10.5 mg, 0.1 mmol) and Et₃SiH (126 mg, 1.08 mmol) in 3.2 mL of CH₂Cl₂ was stirred for 15 h at 25° C., then filtered over Celite and the Celite was washed with CH₂Cl₂ (5 mL). The organic solution was treated with 1 M Bu₄NF in THF (0.8 mL, 0.8 mmol) and after 2 min at 25° C., quenched with the addition of saturated NH₄Cl (15 mL). The reaction mixture was extracted with CH₂Cl₂ (3×25 mL), the combined organic layers were washed with saturated NaCl (10 mL), dried (Na₂SO₄), and concentrated in vacuo. Flash chromatography (2.5×25 cm, SiO₂, 17% EtOAc/hexane) afforded ethyl N-tert-butyloxy-carbonyl-5-hydroxyindole-2-carboxylate 110 (109 mg, 46%) as a white solid: mp 93-94° C.; MALDIFT-HRMS m/z 328.1155 (M+Na⁺, C₁₆H₁₉NNaO₅ requires 328.1155).

A solution of ethyl N-tert-butyloxycarbonyl-5-hydroxyindole-2-carboxylate 110 (58 mg, 0.19 mmol) in 0.6 mL of DMF was treated with NaH (60%, 8 mg, 0.2 mmol) at 25° C. After stirring for 15 min at 25° C., bromoethane was added and the reaction mixture was stirred for 18 h at 25° C. Saturated NaCl (5 mL) was added and the reaction mixture was extracted with EtOAc (2×20 mL), the combined organic layers were washed with saturated NaCl (3×5 mL), dried (Na₂SO₄), and concentrated in vacuo. Flash chromatography (1×20 cm, SiO₂, 9% EtOAc/hexane) afforded ethyl N-tert-butyloxycarbonyl-5-ethoxyindole-2-carboxylate 111 (40 mg, 63%) as an oil: ¹H NMR (acetone-d₆, 500 MHz) δ 7.96 (1H, d, J=9.0 Hz), 7.16 (1H, d, J=2.6 Hz), 7.10 (1H, s), 7.06 (1H, dd, J=9.0, 2.6 Hz), 4.36 (2H, q, J=7.1 Hz), 4.07 (2H, q, J=6.8 Hz), 1.62 (9H, s), 1.35-1.40 (6H, m,); MALDIFT-HRMS m/z 356.1477 (M+Na⁺, C₁₈H₂₃NNaO₅ requires 356.1468).

Ethyl N-tert-butyloxycarbonyl-5-ethoxyindole-2-carboxylate 111 (39 mg, 0.117 mmol) was hydrolyzed with 3 N KOH (10 mL) in EtOH (1 mL) for 0.5 h to give 112 (11.3 mg, 47%) as a pale orange solid after crystallization (EtOAc/hexane): mp 180° C. (lit.(Patent: WO 9109849) mp 202-203° C.); ¹H NMR (DMSO-d₆, 500 MHz) δ 12.77 (1H, br s), 11.56 (1H, s), 7.31 (1H, d, J=9.3 Hz), 7.07 (1H, d, J=2.4 Hz), 6.97 (1H, m), 6.88 (1H, dd, J=8.8, 2.5 Hz), 4.00 (2H, q, J=7.0 Hz), 1.33 (3H, t, J=6.9 Hz).

The propyl and butyl homologues were synthesized following the procedure described for 112. Ethyl N-tert-butyloxycarbonyl-5-propyloxyindole-2-carboxylate (33 mg, 52 mg theoretical, 63%) as an oil: ¹H NMR (acetone-d₆, 500 MHz) δ 7.95 (1H, dd, J=8.6, 0.9 Hz), 7.18 (1H, d, J=2.6 Hz), 7.10 (1H, d, J=0.9 Hz), 7.07 (1H, dd, J=9.0, 2.6 Hz), 4.36 (2H, q, J=7.3 Hz), 3.98 (2H, t, J=6.6 Hz), 1.77-1.84 (2H, m), 1.62 (9H, s), 1.37 (3H, t, J=7.3 Hz), 1.04 (3H, t, J=7.5 Hz).

Ethyl N-tert-butyloxycarbonyl-5-butyloxyindole-2-carboxylate (46 mg, 54 mg theoretical, 85%) as an oil: ¹H NMR (acetone-d₆, 500 MHz) δ 7.96 (1H, dm, J=9.0 Hz), 7.18 (1H, d, J=2.6 Hz), 7.10 (1H, s), 7.07 (1H, dd, J=9.0, 2.6 Hz), 4.36 (2H, q, J=7.1 Hz), 4.02 (2H, t, J=6.4 Hz), 1.74-1.80 (2H, m), 1.62 (9H, s), 1.48-1.55 (2H, m), 1.37 (3H, t, J=7.5 Hz), 0.97 (3H, t, J=7.5 Hz).

5-Propyloxyindole-2-carboxylic acid (113): (15 mg, 21.9 mg theoretical, 68%) as a beige solid crystallized from EtOAc/hexane: mp 155° C.; MALDIFT-HRMS m/z 219.0894 (M⁺, C₁₂H₁₃NO₃ requires 219.0895).

5-Butyloxyindole-2-carboxylic acid (114): (18 mg, 23.3 mg theoretical, 77%) as a beige solid crystallized from EtOAc/hexane: mp 153° C.; MALDIFT-HRMS m/z 233.1060 (M⁺, C₁₃H₁₅NO₃ requires 233.1052).

5- and 7-Trifluoromethoxyindole-2-carboxylic acids (118, 119): 3-trifluoromethoxy-benzaldehyde 115 (380 mg, 2 mmol) and methyl azidoacetate (691 mg, 6 mmol) in MeOH (4 mL) were treated at −20° C. with sodium methoxide solution generated in situ from Na (138 mg, 6 mmol) and 4 mL of MeOH. The reaction mixture was allowed to warm over 1 h at 25° C. and stirred for 2 h at this temperature, then quenched with the addition of ice water (20 mL) and extracted with EtOAc (2×100 mL). The combined organic layers were washed with saturated NaCl (20 mL), dried (Na₂SO₄), and concentrated in vacuo. Flash chromatography (1.5×25 cm, SiO₂, 9% EtOAc/hexane) afforded methyl 2-azido-3-(3-trifluoromethoxyphenyl)acrylate (272 mg, 47%) as a yellow oil: ¹H NMR (CDCl₃, 500 MHz) δ 7.74 (1H, s), 7.68 (1H, dm, J=7.7 Hz), 7.41 (1H, t, J=7.9 Hz), 7.19 (1H, dm, J=8.4 Hz), 6.86 (1H, s); 3.93 (3H, s).

Methyl 2-azido-3-(3-trifluoromethoxyphenyl)acrylate (272 mg, 0.95 mmol) in m-xylene (20 mL) was stirred for 30 min at 135° C., then concentrated in vacuo. Flash chromatography (1.5×25 cm, SiO₂, 14% EtOAc/hexane) afforded methyl 5-trifluoromethoxyindole-2-carboxylate 116 (53 mg, 22%) as a white solid: mp 165° C.; ESI (pos.) m/z 260 (M+H⁺, C¹¹H₉F₃NO₃); ESI (negative) m/z 258 (M−H⁻, C₁₁H₇F₃NO₃); and methyl 7-trifluoromethoxy-indole-2-carboxylate (117) (32 mg, 13%) as a white solid: mp 129° C.; ESI (negative) m/z 294 (M+Cl⁻, C₁₁H₈ClF₃NO₃), 258 (M−H⁻, C₁₁H₇F₃NO₃).

116 (25.9 mg, 0.1 mmol) in dioxane/H₂O (4:1, 500 μL) was treated with 4 M LiOH (100 μL) for 15 h at 25° C., then quenched with the addition of 1 M HCl (1 mL) and ice, and extracted with EtOAc (3×15 mL). The combined organic layers were dried (Na₂SO₄), and concentrated in vacuo to afford 118 (24 mg, 98%) as a white solid: mp 175-176° C.; ESI (negative) m/z 244 (M−H⁻, C₁₀H₅F₃NO₃).

117 (53 mg, 0.2 mmol) in dioxane/H₂O (4:1, 1 mL) was treated with 4 M LiOH (200 μL) for 12 h at 25° C., then quenched with the addition of 1 M HCl (1 mL) and ice, and extracted with EtOAc (2×20 mL). The combined organic layers were dried (Na₂SO₄), and concentrated in vacuo. Crystallization from THF/hexane afforded 119 (30 mg, 60%) as an off-white solid: mp 194° C.; IR (film) vₘₐₓ 3453, 2875, 1673, 1540, 1252, 1221, 1198, 1165, 883 cm⁻¹.

5- and 7-Thiomethylindole-2-carboxylic acid (125a, 125b): 2-(3-thiomethylphenyl)-[1,3]dioxolane (Guspanová, L.; et al. Helv. Chim. Acta 1997, 80, 1375) (500 mg, 2.56 mmol) was treated with 1 N HCl (5.0 mL) in THF (25 mL) for 2 h to give 3-thiomethylbenzaldehyde 121 (332 mg, 85%) as a colorless oil: ¹H NMR (CDCl₃, 500 MHz) δ 9.96 (1H, s), 7.72 (1H, m), 7.59-7.61 (1H, m), 7.47-7.49 (1H, m), 7.42 (1H, t, J=7.7 Hz), 3.52 (3H, s).

121 (299 mg, 1.96 mmol) was treated with methyl azidoacetate and thermally converted to the indole following the same procedure as for 102 to give methyl 5-thiomethylindole-2-carboxylate 123 (45 mg, 25%) as a pale yellow solid: mp 135° C.; MALDIFT-HRMS m/z 221.0506 (M⁺, C₁₁H₁₁NO₂S requires 221.0505); and methyl 7-thiomethylindole-2-carboxylate 124 (66 mg, 36%) as a pale yellow solid: mp 81° C.; MALDIFT-HRMS m/z 221.0506 (M⁺, C₁H₁₁NO₂S requires 221.0505).

123a (33.2 mg, 0.15 mmol) in dioxane/H₂O (4:1, 1 mL) was treated with 4 M LiOH (200 μL) for 15 h at 25° C., then quenched with the addition of 1 M HCl (2 mL) and ice, and extracted with EtOAc (2×20 mL). The combined organic layers were dried (Na₂SO₄), and concentrated in vacuo to afford 125a (31.0 mg, quant.) as a white solid: mp 240° C. dec.; MALDIFT-HRMS m/z 207.0349 (M⁺, C₁₀H₉NO₂S requires 207.0349).

124a (44.3 mg, 0.2 mmol) in dioxane/H₂O (4:1, 1 mL) was treated with 4 M LiOH (200 μL) for 15 h at 25° C., then quenched with the addition of 1 M HCl (2 mL) and ice, and extracted with EtOAc (2×20 mL). The combined organic layers were dried (Na₂SO₄), and concentrated in vacuo to afford 125b (40 mg, 97%) as an off-white solid: mp 161-162° C.; MALDIFT-HRMS m/z 207.0350 (M⁺, C₁₀H₉NO₂S requires 207.0349).

5-Thioethylindole-2-carboxylic acid (126a): According to literature procedures (Ornstein, P. L.; et al. J. Med. Chem. 1998, 41, 358-378), 2-(3-bromophenyl)-[1,3]dioxolane 120 (2.75 g, 12 mmol) in 20 mL of THF was treated with a 2 M solution of BuLi in hexane (6 mL, 12 mmol) at −78° C. After 10 min, diethyldisulfide (1.47 g, 12 mmol) was added. The reaction mixture was stirred for 1 h at −78° C., then allowed to warm to 0° C. over 1 h, and then diluted with EtOAc (100 mL). The organic layer was washed with saturated NH₄Cl (20 mL), saturated NaCl (20 mL), and then dried (Na₂SO₄), and concentrated in vacuo. Flash chromatography (3.5×15 cm, SiO₂, 5% EtOAc/hexane) afforded 2-(3-thioethyl-phenyl)-[1,3]dioxolane (1.96 g, 78%) as a colorless oil: ¹H NMR (CDCl₃, 500 MHz) δ 7.44 (1H, s), 7.26-7.33 (3H, m), 5.79 (1H, s), 4.00-4.15 (4H, m), 2.96 (2H, q, J=7.5 Hz), 1.31 (3H, t, J=7.3 Hz); MALDIFT-HRMS m/z 211.0785 (M+H⁺, C₁₁H₁₅O₂S requires 211.0787).

2-(3-Thioethylphenyl)-[1,3]dioxolane (1.26 g, 6 mmol) in 10 mL THF was treated with 1 M HCl (10 mL), and then extracted with EtOAc (2×200 mL). The combined organic layers were washed with saturated NaCl (2×20 mL), dried (Na₂SO₄), and concentrated in vacuo. Flash chromatography (2.5×20 cm, SiO₂, 5% EtOAc/hexane) 3-thioethylbenzaldehyde 122 (592 mg, 59%, 67% based on recovered starting material) as a colorless oil: ¹H NMR (CDCl₃, 500 MHz) δ 9.98 (1H, s), 7.79 (1H, t, J=1.8 Hz), 7.65 (1H, dt, J=7.5, 1.3 Hz), 7.55 (1H, ddd, J=7.9, 2.1, 1.2 Hz), 7.44 (1H, t, J=7.6 Hz), 3.01 (2H, q, J=7.3 Hz), 1.35 (3H, t, J=7.3 Hz).

122 (499 mg, 3 mmol) and methyl azidoacetate (1.38 g, 12 mmol) in 4 ml MeOH were treated at −5° C. with a sodium methoxide solution generated in situ with Na (276 mg, 12 mmol) and 5 mL of MeOH. The reaction mixture was stirred for 1 h at −5° C. and then allowed to warm over 2 h at 25° C., before being quenched with the addition of ice water (20 mL) and extracted with EtOAc (2×100 mL). The combined organic layers were dried (Na₂SO₄), and concentrated in vacuo. Flash chromatography (2×20 cm, SiO₂, 9% EtOAc/hexane) afforded methyl 2-azido-3-(3-thioethylphenyl)acrylate (543 mg, 69%) as a pale yellow oil.

2-Azido-3-(3-thioethylphenyl)acrylate (540 mg, 2.05 mmol) in m-xylene (30 mL) was stirred for 3 h at 130° C., then concentrated in vacuo. Flash chromatography (2×20 cm, SiO₂, 5% to 9% EtOAc/hexane) afforded methyl 5-thioethylindole-2-carboxylate 123b (146 mg, 30%) as an off-white solid: mp 115-116° C.; MALDIFT-HRMS m/z 235.0665 (M⁺, C₁₂H₁₃NO₂S requires 235.0667); and methyl 7-thioethylindole-2-carboxylate 124b (222 mg, 46%) as an off-white solid: mp 82-83° C.

124b (47 mg, 0.2 mmol) in dioxane/H₂O (4:1, 1 mL) was treated with 4 M LiOH (200 μL) for 12 h at 25° C., then quenched with the addition of 1 M HCl (2 mL) and ice, and extracted with EtOAc (2×20 mL). The combined organic layers were dried (Na₂SO₄), and concentrated in vacuo to afford 126b (43 mg, 97%) as a white solid: mp 222° C. (dec.); MALDIFT-HRMS m/z 221.0503 (M⁺, C₁₁H₁₁NO₂S requires 221.0505).

5-Ethylsulfonylindole-2-carboxylic acid (127): 124 (47 mg, 0.2 mmol) was treated with m-CPBA 68% (107 mg, 0.42 mmol) in 1 mL of CH₂CO₂ at −78° C. The reaction mixture was allowed to warm to 25° C. over 3 hrs, then quenched with saturated NaCl (15 mL) and extracted with CH₂Cl₂ (3×50 mL). The combined organic layers were dried (Na₂SO₄), and concentrated in vacuo. Flash chromatography (2×20 cm, SiO₂, 25% to 40% hexane/EtOAc) afforded methyl 7-ethyl-sulfonylindole-2-carboxylate (43.5 mg, 81%) as a white solid: mp 149° C.; MALDIFT-HRMS m/z 290.0458 (M+Na⁺, C₁₂H₁₃NNaO₄S requires 290.0457).

Methyl 7-ethylsulfonylindole-2-carboxylate (26.7 mg, 0.1 mmol) in dioxane/H₂O (4:1, 500 μL) was treated with 4 M LiOH (100 μL) for 18 h at 25° C., then quenched with the addition of 1 M HCl (5 mL) and ice, and extracted with EtOAc (3×20 mL). The combined organic layers were dried (Na₂SO₄), and concentrated in vacuo to afford 127 (25.2 mg, 97%) as an off-white solid: mp 286° C. (dec.); MALDIFT-HRMS m/z 276.0301 (M+Na⁺, C₁₁H₁₁NNaO₄S requires 276.0301).

5- and 7-Methoxymethylindole-2-carboxylic acids (132, 133): 128 (354 mg, 2 mmol) and NaBH₄ (76 mg, 2 mmol) in 10 ml of MeOH were stirred for 15 min at 0° C., then quenched with acetone (1 mL) and concentrated in vacuo. The residue was dissolved in EtOAc (40 mL) and washed with H₂O (10 mL). The aqueous layer was extracted with EtOAc (40 mL). The combined organic layers were dried (Na₂SO₄), and concentrated in vacuo to afford crude 3-[1,3]dioxolan-2-ylbenzyl alcohol as a colorless oil: ¹H NMR (CDCl₃, 400 MHz) δ 7.49 (1H, s), 7.36-7.42 (4H, m), 5.81 (1H, s), 4.70 (2H, d, J=4.1 Hz), 4.00-4.17 (4H, m); which was treated with 60% NaH (88 mg, 2.2 mol) in 8 mL THF at 0° C. After stirring for 30 min at 0° C., MeI (426 mg, 3 mmol) was added, and the reaction mixture was allowed to warm over 14 h to 25° C., then quenched with 5% HCl (4 mL). The reaction mixture was stirred for 30 min at 25° C., and then diluted with H₂O (15 mL) and extracted with EtOAc (2×50 mL). The combined organic layers were washed with saturated NaCl (10 mL), dried (Na₂SO₄), and concentrated in vacuo. Flash chromatography (1.5×20 cm, SiO₂, 9% EtOAc/hexane) afforded 3-methoxymethylbenzaldehyde 129 (180 mg, 46% overall) as a colorless oil: ¹H NMR (CDCl₃, 400 MHz) δ 10.03 (1H, s), 7.86 (1H, m), 7.81 (1H, dm, J=7.6 Hz), 7.62 (1H, dm, J=7.6 Hz), 7.53 (1H, t, J=7.5), 4.54 (2H, s), 3.43 (3H, s).

129 (215 mg, 1.43 mmol) and methyl azidoacetate (494 mg, 4.29 mmol) in MeOH (3 mL) were treated at −20° C. with sodium methoxide solution generated in situ with Na (99 mg, 4.29 mmol) and 3 mL of MeOH. The reaction mixture was allowed to warm over 1 h to 25° C. and stirred for 2 h at this temperature, then quenched with the addition of ice water (20 mL) and extracted with EtOAc (2×80 mL). The combined organic layers were washed with saturated NaCl (20 mL), dried (Na₂SO₄), and concentrated in vacuo. Flash chromatography (1.5×25 cm, SiO₂, 9% EtOAc/hexane) afforded methyl 2-azido-3-(3-methoxymethylphenyl)acrylate as an oil.

Methyl 2-azido-3-(3-methoxymethylphenyl)acrylate was stirred in m-xylene (10 mL) for 3 h at 135° C., then concentrated in vacuo. Flash chromatography (1.5×25 cm, SiO₂, 9% EtOAc/hexane) afforded 130 (26 mg, 8%) as a white solid: mp 91-92° C.; MALDIFT-HRMS m/z 219.0886 (M+H⁺, C₁₂H₁₃NO₃ requires 219.0890); and 131 (23 mg, 7%) as a white solid: mp 124° C.; MALDIFT-HRMS m/z 242.0798 (M+Na⁺, C₁₂H₁₃NNaO₃ requires 242.0788).

130 (22 mg, 0.1 mmol) in dioxane/H₂O (4:1, 550 μL) was treated with 4 M LiOH (55 μL, 0.22 mmol) for 9 h at 25° C., then quenched with the addition of 1 M HCl (1.5 mL), and extracted with EtOAc (2×15 mL). The combined organic layers were dried (Na₂SO₄), and concentrated in vacuo. Preparative TLC (20×20, THF) afforded 132 (17 mg, 83%) as a white solid: mp 195° C.; MALDIFT-HRMS m/z 205.0743 (M⁺, C₁₁H₁₁NO₃ requires 205.0739).

131 (25 mg, 0.11 mmol) in dioxane/H₂O (4:1, 550 μL) was treated with 4 M LiOH (55 μL, 0.22 mmol) for 9 h at 25° C., then quenched with the addition of 1 M HCl (1.5 mL), and extracted with EtOAc (2×15 mL). The combined organic layers were dried (Na₂SO₄), and concentrated in vacuo. Preparative TLC (20×20, THF) afforded 133 (18 mg, 77%) as a white solid: mp 196° C. (dec.); ESI (neg.) m/z 204 (M−H⁻, C₁₁H₁₀NO₃).

5-Azidoindole-2-carboxylic acid (x): A suspension of ethyl 5-nitroindole-2-carboxylate 134 (800 mg, 3.4 mmol) and 10% Pd/C (160 mg) in 70 mL of EtOAc was stirred for 14 h under 1 atm H₂. The catalyst was removed by filtration through Celite and washed with EtOAc (50 mL). Evaporation of the solvent afforded ethyl 5-aminoindole-2-carboxylate (667 mg, 96%) as a beige solid: mp 124-125° C. (lit. (Boger, D. L.; et al. J. Org. Chem. 1987, 53, 1521-1530) 127-127.5° C.).

A suspension of the crude amine (408 mg, 2 mmol) in 3 mL of HCl (16%) was cooled in an ice-bath and then treated with a solution of NaNO₂ (242 mg, 3.5 mmol) in 800 μL of water for 30 min. The resulting solution was added to an ice-bath cooled solution of NaN₃ (260 mg, 4 mmol) and NaOAc (1.64 g, 20 mmol) in 6 mL of H₂O. The foamy reaction mixture was stirred for 30 min at 0° C. and for 1 h at 25° C., then diluted with 20 mL of H₂O and extracted with CHCl₃ (2×30 mL). The combined organic layers were dried (Na₂SO₄), concentrated in vacuo and crystallized (EtOAc/hexane) to afford ethyl 5-azidoindole-2-carboxylate 135 (290 mg, 63%) as a beige solid: mp 140° C. (lit. (Bu'Lock, J. D.; Harley-Mason, J. J. Chem. Soc., 1951, 703-712) mp 141-143° C.).

Ethyl 5-azidoindole-2-carboxylate 135 (46 mg, 0.2 mmol) was hydrolyzed following the procedure described for 132 to give 136 (40 mg, 100%) as a solid: mp 150° C. (dec); MS (ESI negative) m/z 237 (M+Cl⁻), 201 (M−H⁻).

5-Cyanoindole-2-carboxylic acid (140): A suspension of CuCN (27 mg, 0.3 mmol) and ethyl 5-bromoindole-2-carboxylate 138 in 500 μL of DMF was stirred at 155° C. for 7 h. Chromatography (3×15 cm, SiO₂, 20% EtOAc/hexane) gave ethyl 5-cyanoindole-2-carboxylate 139 (34 mg, 63%) as a white solid: mp 181-182° C. (lit. (Boger, D. L.; et al. J. Org. Chem. 1987, 53, 1521-1530) mp 185.5-187° C.).

Ethyl 5-cyanoindole-2-carboxylate 139 (21.4 mg, 0.1 mmol) was hydrolyzed following the procedure described for 112-114 to give 140 (13 mg, 70%) as a white solid after crystallization (EtOAc): mp 320° C. (dec) (lit. (Zheng, Y.-J; Merz, Jr., K. M. J. Am. Chem. Soc. 1992, 114, 10498-10507) mp 315-330° C. (dec)).

7-Cyanoindole-2-carboxylic acid (144): Conversion of ethyl 7-bromoindole-2-carboxylate 142 by the procedure described for 138 provided ethyl 7-cyanoindole-2-carboxylate 143 (34 mg, theoretical 53.6 mg, 63%) as a white solid: mp 148-149° C.

Ethyl 7-cyanoindole-2-carboxylate (21.4 mg, 0.1 mmol) was hydrolyzed following the procedure described for 112-114 to give 144 (14.9 mg, 80%) as a white solid: mp 260° C. (dec); MALDIFT-HRMS m/z 185.0365 (M−H⁻, C₁₀H₅N₂O₂ requires 185.0357).

5-Vinylindole-2-carboxylic acid (51): 148 (124 mg, 0.5 mmol) in 3 mL of THF was treated with 5% HCl (1.5 mL). The reaction mixture was stirred for 30 min at 25° C., then diluted with saturated NaCl (10 mL), and extracted with EtOAc (2×50 mL). The combined organic layers were washed with saturated NaCl (10 mL), dried (Na₂SO₄), and concentrated in vacuo. Flash chromatography (1.5×15 cm, SiO₂, 17% EtOAc/hexane) afforded methyl 5-formylindole-2-carboxylate (58 mg, 57%) as a white solid: mp 208° C.; MALDIFT-HRMS m/z 204.0658 (M+H⁺, C₁₁H₁₀NO₃ requires 204.0655).

Ph₃PCH₃I (275 mg, 0.68 mmol) in 2 mL of THF was treated at 0° C. with 0.5 M KHMDS in toluene (1.43 mL, 0.715 mmol). The reaction mixture was stirred for 30 min at 0° C., then methyl 5-formylindole-2-carboxylate (46 mg, 0.23 mmol) in THF (3 mL) was added. The reaction mixture was allowed to warm over 8 h to 25° C., then quenched with saturated NH₄Cl (15 mL), and extracted with EtOAc (2×30 mL). The combined organic layers were dried (Na₂SO₄), and concentrated in vacuo. Flash chromatography (1.5×20 cm, SiO₂, 9% EtOAc/hexane) afforded 149 (32 mg, 70%) as a white solid: mp 142-143° C.; MALDIFT-HRMS m/z 202.0862 (M+H⁺, C₁₂H₁₂NO₂ requires 202.0863).

149 (20.1 mg, 0.1 mmol) in dioxane/H₂O (4:1, 500 μL) was treated with 4 M LiOH (50 μL, 0.2 mmol) for 12 h at 25° C., then quenched with the addition of 1 M HCl (1 mL) and H₂O (2 mL), and extracted with EtOAc (2×15 mL). The combined organic layers were dried (Na₂SO₄), and concentrated in vacuo to afford 151 (18.5 mg, 99%) as a white solid: mp 240° C. dec.; MALDIFT-HRMS m/z 188.0710 (M+H⁺, C₁₁H₁₀NO₂ requires 188.0706).

7-Vinylindole-2-carboxylic acid (47): 145 (95 mg, 0.5 mmol) in 5 mL of THF was treated with 0.4 M CH₂N₂ in Et₂O (1.25 mL, 0.5 mmol), and then evaporated. Filtration through a plug of silica gel (EtOAc/hexane) afforded methyl 7-formylindole-2-carboxylate (98 mg, 96%) as a white solid: mp 87° C.; MALDIFT-HRMS m/z 204.0657 (M+H⁺, C₁₁H₁₀NO₃ requires 204.0655). Ph₃PCH₃I (477 mg, 1.18 mmol) in 4 mL of THF was treated at 0° C. with 0.5 M KHMDS in toluene (2.5 mL, 1.25 mmol). The reaction mixture was stirred for 30 min at 0° C., then methyl 7-formylindole-2-carboxylate (80 mg, 0.39 mmol) in THF (1 mL) was added. The reaction mixture was allowed to warm over 8 h to 25° C., then quenched with saturated NH₄Cl (20 mL), and extracted with EtOAc (2×50 mL). The combined organic layers were dried (Na₂SO₄), and concentrated in vacuo. Flash chromatography (1.5×20 cm, SiO₂, 9% EtOAc/hexane) afforded 146 (70 mg, 88%) as a white solid: mp 121-122° C.; MALDIFT-HRMS m/z 202.0868 (M+H⁺, C₁₂H₁₂NO₂ requires 202.0863).

146 (30.2 mg, 0.15 mmol) in dioxane/H₂O (4:1, 750 μL) was treated with 4 M LiOH (75 μL, 0.3 mmol) for 12 h at 25° C., then quenched with the addition of 1 M HCl (1.5 mL) and H₂O (2 mL), and extracted with EtOAc (2×15 mL). The combined organic layers were dried (Na₂SO₄), and concentrated in vacuo. Flash chromatography (1.5×20 cm, SiO₂, 10% MeOH/CH₂Cl₂) afforded 147 (23 mg, 82%) as a white solid: mp 185° C. (dec.); MALDIFT-HRMS m/z 188.0709 (M+H⁺, C₁₁H₁₀NO₂ requires 188.0706).

5-Ethylindole-2-carboxylic acid (150): 149 (10 mg, 0.05 mmol) and Pd/C (2.5 mg) in 500 μL of THF were stirred under H₂ (1 bar) for 1 h at 25° C., then filtered over Celite. The catalyst was washed with THF (1 mL) and the filtrate was evaporated to afford methyl 5-ethylindole-2-carboxylate (10 mg, 99%) as a white solid: mp 112° C.; MALDIFT-HRMS m/z 204.1023 (M+H⁺, C₁₂H₁₄NO₂ requires 204.1019).

Methyl 5-ethylindole-2-carboxylate (9 mg, 0.044 mmol) and 87% KOH (11.5 mg, 0.177 mmol) in 300 μL of EtOH was stirred for 30 min at 80° C., then quenched with 1 M HCl (1.5 mL), and extracted with EtOAc (2×15 mL). The combined organic layers were dried (Na₂SO₄), and concentrated in vacuo to afford 150 (8 mg, 95%) as an off-white solid: mp 173° C. (lit. (Matsuhashi, H.; et al. Bull. Chem. Soc. Jpn. 1997, 70, 437-444) 184° C.).

5-Isopropenylindole-2-carboxylic acid (153): Ph₃PCH₃I (477 mg, 1.18 mmol) in 5 mL of THF was treated at 0° C. with 0.5 M KHMDS in toluene (2.5 mL, 1.24 mmol). The reaction mixture was stirred for 30 min at 0° C., then 152 (91 mg, 0.39 mmol) in THF (1 mL) was added. The reaction mixture was allowed to warm over 8 h to 25° C., then quenched with saturated NH₄Cl (20 mL), and extracted with EtOAc (2×50 mL). The combined organic layers were washed with saturated NaCl (20 mL), dried (Na₂SO₄), and concentrated in vacuo. Flash chromatography (1.5×15 cm, SiO₂, 4% EtOAc/hexane) afforded ethyl 5-isopropenylindole-2-carboxylate (82 mg, 91%) as a white solid: mp 124-125° C.; MALDIFT-HRMS m/z 230.1173 (M+H⁺, C₁₄H₁₆NO₂ requires 230.1175).

Ethyl 5-isopropenylindole-2-carboxylate (46 mg, 0.2 mmol) in dioxane/H₂O (4:1, 1 mL) was treated with 4 M LiOH (100 μL, 0.4 mmol) for 15 h at 25° C., then quenched with the addition of 1 M HCl (2 mL) and H₂O (4 mL), and extracted with EtOAc (2×25 mL). The combined organic layers were dried (Na₂SO₄), and concentrated in vacuo to afford 153 (40 mg, quant.) as a white solid: mp 205° C. (dec.); MALDIFT-HRMS m/z 202.0862 (M+H⁺, C₁₂H₁₂NO₂ requires 202.0863).

7-Isopropenylindole-2-carboxylic acid (155): Using the same procedure as used for 153, 154 (91 mg, 0.39 mmol) was converted to ethyl 7-isopropenylindole-2-carboxylate (77 mg, 86%) as a colorless oil: MALDIFT-HRMS m/z 230.1177 (M+H⁺, C₁₄H₁₆NO₂ requires 230.1175).

Ethyl 7-isopropenylindole-2-carboxylate (46 mg, 0.2 mmol) in dioxane/H₂O (4:1, 1 mL) was treated with 4 M LiOH (100 μL, 0.4 mmol) for 24 h at 25° C., then quenched with the addition of 1 M HCl (2 mL) and H₂O (4 mL), and extracted with EtOAc (2×25 mL). The combined organic layers were dried (Na₂SO₄), and concentrated in vacuo to afford 155 (32.5 mg, 81%) as a white solid: mp 167-168° C. (dec.); MALDIFT-HRMS m/z 202.0862 (M+H⁺, C₁₂H₁₂NO₂ requires 202.0863).

5-Ethynylindole-2-carboxylic acid (157): A suspension of ethyl 5-bromoindole-2-carboxylate 138 (80 mg, 0.30 mmol), (Ph₃P)₄Pd (34.5 mg, 0.03 mmol), CuI (7.7 mg, 0.04 mmol), trimethyl-silylacetylene (59 mg, 0.60 mmol) in 600 μL of CH₃CN and 1.5 mL of Et₃N was stirred for 5 h at 80° C., then filtered over Celite and washed with EtOAc. After concentration, the filtrate treated to flash chromatography (1.5×25 cm, 10% EtOAc/hexane) which afforded an inseparable mixture of ethyl 5-(trimethylsilylacetylenyl)indole-2-carboxylic acid and starting material (85:15 by ¹H NMR) (73 mg, 74%) as a white solid: ¹H NMR (CDCl₃, 250 MHz) δ 8.87 (1H, br s), 7.85 (1H, s), 7.32-7.44 (2H, m), 7.18-7.19 (1H, m), 4.41 (2H, q, J=7.2 Hz), 1.41 (3H, t, J=7.1 Hz), 0.26 (9H, s).

A solution of this mixture (70 mg, 0.21 mmol) in 3 mL of EtOH was treated with 0.2 M NaOH (1 mL) for 20 min at 25° C. then quenched with the addition of 10% citric acid (1 mL). The reaction mixture was partially evaporated, diluted with saturated NaCl (10 mL) and extracted with Et₂O (3×20 mL). The combined organic layers were dried (Na₂SO₄), and concentrated in vacuo. The residue was dry loaded onto silica gel and flash chromatography (1.5×30 cm, 8% EtOAc/hexane) afforded an inseparable mixture of ethyl 5-(acetylenyl)indole-2-carboxylic acid and ethyl 5-bromoindole-2-carboxylate 138 (85:15 by ¹H NMR) (32 mg, 59%) as a white solid: ¹H NMR (CDCl₃, 500 MHz) δ 9.23 (1H, br s), 7.88 (1H, m), 7.43 (1H, dd, J=8.4, 1.5 Hz), 7.37 (1H, dm, J=8.4 Hz), 7.20-7.22 (1H, m), 4.44 (2H, q, J=7.1 Hz), 1.43 (3H, t, J=7.2 Hz); ¹³C NMR (CDCl₃, 125 MHz) δ 161.8, 136.5, 129.0, 128.5, 127.1, 124.9, 114.3, 112.0, 108.6, 84.5, 75.5, 61.3, 14.3.

A solution of this mixture (28 mg, 0.11 mmol), Boc₂O (58 mg, 0.26 mmol) and DMAP (16 mg, 0.13 mmol) in 2 mL of CH₂Cl₂ was stirred for 1 h at 25° C. then quenched with the addition of 0.2 M HCl saturated with NaCl (5 mL). The reaction mixture was extracted with CH₂Cl₂ (3×20 mL), the combined organic layers were dried (Na₂SO₄), and concentrated in vacuo. Flash chromatography (1.5×25 cm, SiO₂, 3% EtOAc/hexane) afforded ethyl 5-acetylenyl-N-(tert-butyloxycarbonyl)indole-2-carboxylate 156 (30 mg, 89%) as an oil: ¹H NMR (CDCl₃, 500 MHz) δ 8.03 (1H, d, J=8.8 Hz), 7.75 (1H, d, J=1.7 Hz), 7.51 (1H, dd, J=8.6, 1.7 Hz), 7.05 (1H, s), 4.38 (2H, q, J=7.1 Hz), 3.05 (1H, s), 1.63 (9H, s), 1.39 (3H, t, J=7.2 Hz); ¹³C NMR (CDCl₃, 125 MHz) δ 161.5, 148.9, 137.4, 131.8, 130.4, 127.4, 126.2, 117.0, 114.9, 113.9, 85.0, 83.7, 76.3, 61.5, 27.8, 14.2; IR (film) vₘₐₓ 3289, 2981, 1738, 1732, 1460, 1394, 1372, 1359, 1338, 1321, 1290, 1260, 1233, 1207, 1158, 1135, 1122, 1072 cm⁻¹; MS (ESI positive) m/z 336 (M+Na⁺).

A solution of ethyl 5-acetylenyl-N-(tert-butyloxycarbonyl)indole-2-carboxylate 156 (32 mg, 0.10 mmol) in 1 mL of dioxane/H₂O (4:1) was treated with 4 M LiOH (100 μL) and the mixture was stirred for 48 h at 25° C. 1 M HCl (2 mL) was added, and the mixture was extracted with EtOAc (3×5 mL). The combined organic layers were dried (Na₂SO₄), and concentrated in vacuo. Flash chromatography (1×20 cm, 5% MeOH/CH₂Cl₂) afforded 157 (11 mg, 59%) as a beige solid: mp 150° C. (dec.); MALDIFT-HRMS m/z 186.0542 (M+H⁺, C₁₁H₈NO₂ requires 186.0550).

5-(1-Propynyl)-indole-2-carboxylic acid (159): A suspension of ethyl 5-bromoindole-2-carboxylate 138 (90 mg, 0.375 mmol), Pd(PhCN)₂Cl₂ (90 mg, 0.03 mmol), CuI (4.0 mg, 0.02 mmol) in 2 mL of dioxane was saturated with propyne and ^{t}Bu₃P (12.1 mg, 0.06 mmol) and diisopropylamine (45.5 mg, 0.45 mmol) were added. The reaction mixture was stirred for 48 h at 25° C. and filtered (2.5×20 cm, SiO₂, 10% EtOAc/hexane) to give a mixture of product and starting material (1:1 by ¹H NMR) (79 mg). The mixture (45 mg) was subjected again to the reaction conditions and flash chromatography (1.5×20 cm, SiO₂, 10% EtOAc/hexane) afforded ethyl 5-(1-propynyl)-indole-2-carboxylate 158 (35 mg, 72%) as a beige solid: mp 166-167° C.; MALDIFT-HRMS m/z 228.1027 (M+H⁺, C₁₄H₁₄NO₂ requires 228.1019).

A solution of ethyl 5-(1-propynyl)-indole-2-carboxylate 158 (22.7 mg, 0.10 mmol) in 1 mL of dioxane/H₂O (4:1) was treated with 4 M LiOH (100 μL) and stirred for 36 h at 25° C., then quenched with the addition of 1 M HCl (2 mL), and extracted with EtOAc (3×5 mL). The combined organic layers were dried (Na₂SO₄), and concentrated in vacuo. The crude solid was crystallized (CH₂Cl₂/CCl₄) to afford 159 (13 mg, 65%) as a beige solid: mp 237° C. (dec.); MALDIFT-HRMS m/z 200.0709 (M+H⁺, C₁₂H₁₀NO₂ requires 200.0706).

4-Phenylindole-2-carboxylic acid (163): Activated MnO₂ (1.18 g, 13.6 mmol) was suspended in 10 mL of CH₂Cl₂ followed by addition of 2-biphenylmethanol 160 (500 mg, 2.7 mmol). The reaction mixture was stirred for 36 h at 25° C., then filtered over Celite and evaporated. Flash chromatography (1.5×20 cm, SiO₂, 9% EtOAc/hexane) afforded 161 (408 mg, 82%) as a colorless oil: ¹H NMR (CDCl₃, 500 MHz) δ 9.99 (1H, d, J=1.0 Hz), 8.03 (1H, dd, J=7.8, 1.5 Hz), 7.65 (1H, td, J=7.6, 7.6, 1.5 Hz), 7.43-7.52 (5H, m), 7.37-7.52 (2H, m).

Na (184 mg, 8 mmol) was dissolved in 4 mL of MeOH and a solution of 161 (364 mg, 2 mmol) and methyl azidoacetate (921 mg, 8 mmol) in MeOH (3 mL) were added over 1 h at −5° C. The reaction mixture was stirred and allowed to warm over 3 h at 25° C., before being quenched with the addition of ice water (15 mL). The off-white precipitate was filtered, washed with H₂O (15 mL) and dried by azeotropic distillation with benzene to afford methyl 2-azido-3-(2-biphenyl)-acrylate (373 mg, 67%) as a off-white solid: ¹H NMR (CDCl₃, 500 MHz) δ 8.16-8.17 (1H, m), 7.31-7.45 (8H, m), 6.91 (1H, s), 3.80 (3H, s).

A solution of methyl 2-azido-3-(2-biphenyl)acrylate (350 mg, 1.25 mmol) in m-xylene (3 mL) was added over 2 h to refluxing m-xylene (4 mL). The reaction mixture was stirred for 2 h at 140° C., before being concentrated in vacuo. Flash chromatography (dry loaded, 2×20 cm, SiO₂, 17% EtOAc/hexane) afforded 162 (249 mg, 79%) as a white solid: mp 181-182° C.; MALDIFT-HRMS m/z 251.0948 (M⁺, C₁₆H₁₃NO₂ requires 251.0941).

A solution of KOH (87%, 52 mg, 0.8 mmol) and 162 (50.3 mg, 0.2 mmol) in 600 μL of EtOH was stirred for 30 min at 80° C., then quenched with the addition of 1 M HCl (1 mL) and ice, and extracted with EtOAc (2×20 mL). The combined organic layers were dried (Na₂SO₄), and concentrated in vacuo to afford 163 (46.3 mg, 98%) as a off-white solid: mp 243° C. (dec.); MALDIFT-HRMS m/z 237.0786 (M⁺, C₁₅H₁₁NO₂ requires 237.0784).

5-Dimethylaminoindole-2-carboxylic acid (165): 10% Pd/C (10 mg) was placed in a pressure flask and suspended in 1.25 mL of THF. Ethyl 5-nitroindole-2-carboxylate (50 mg, 0.21 mmol) was added followed by the addition of 37% formaldehyde solution (1.50 mL). The flask was purged then placed under H₂ (60 psi, 23° C.) for 24 hour. The catalyst was then filtered over Celite and the filtrate concentrated in vacuo. Flash chromatography of the remaining solid (1×20 cm, SiO₂, 50% EtOAc/hexanes) afforded ethyl 5-dimethylaminoindole-2-carboxylate 164 (35 mg, 71%) as a yellow solid: MALDIFT-HRMS m/z 233.1287 (M+H⁺, C₁₃H₁₇N₂O₂ requires 233.1284).

A solution of ethyl 5-dimethylaminoindole-2-carboxylate 164 (28 mg, 0.12 mmol) in 1.25 mL of THF/MeOH/H₂O (3:1:1) was treated with LiOH (8 mg, 0.19 mmol) and stirred at 23° C. for 3 hours, then neutralized with 1 N HCl and extracted with CH₂Cl₂ (15 mL). The layer was collected and concentrated in vacuo to give 5-dimethylaminoindole-2-carboxylic acid 165 (18 mg, 74%) as an orange solid: IR (film) vₘₐₓ 3456, 1641 cm⁻¹; MALDIFT-HRMS m/z 205.0973 (M+H⁺, C₁₁H₁₃N₂O₂ requires 205.0971).

5-Diethylaminoindole-2-carboxylic acid (167): 10% Pd/C (10 mg) was placed in a pressure flask and suspended in 1.25 mL of THF. Ethyl 5-nitroindole-2-carboxylate (50 mg, 0.21 mmol) was added followed by the addition of acetaldehyde (1.00 mL). The flask was purged then placed under H₂ (60 psi, 23° C.) for 24 hour. The catalyst was then filtered over Celite and the filtrate concentrated in vacuo. Flash chromatography of the remaining solid (1×20 cm, SiO₂, 50% EtOAc/hexanes) afforded ethyl 5-diethylaminoindole-2-carboxylate 166 (41 mg, 74%) as a yellow solid: mp 68-70° C.; MALDIFT-HRMS m/z 261.1588 (M+H⁺, C₁₅H₂₁N₂O₂ requires 261.1597).

A solution of ethyl 5-diethylaminoindole-2-carboxylate 166 (21 mg, 0.08 mmol) in 1.25 mL of THF/MeOH/H₂O (3:1:1) was treated with LiOH (9 mg, 0.22 mmol) and stirred at 23° C. for 3 hours, then neutralized with 1 N HCl and extracted with CH₂Cl₂ (15 mL). The layer was collected and concentrated in vacuo to give 5-diethylaminoindole-2-carboxylic acid 167 (14 mg, 76%) as white solid: MALDIFT-HRMS m/z 233.1280 (M+H⁺, C₁₃H₁₇N₂O₂ requires 233.1284).

5-Propionylaminoindole-2-carboxylic acid (173): Ethyl 5-propionylaminoindole-2-carboxylate crystallized as a gray solid from EtOAc/ⁱPr₂O (32 mg, 104 mg theoretical, 31%): mp 241° C.; MALDIFT-HRMS m/z 261.1237 (M+H⁺, C₁₄H₁₇N₂O₃ requires 261.1234).

A solution of ethyl 5-propionylaminoindole-2-carboxylate (26 mg, 0.1 mmol) in 0.6 mL of EtOH was treated with 2 M Cs₂CO₃ (0.15 mL) for 2.5 h at 80° C., then diluted with H₂O and extracted with CH₂Cl₂ (15 mL). The aqueous layer was acidified with 1 M HCl (1 mL) and extracted with EtOAc (3×15 mL). The combined organic layers were dried (Na₂SO₄), and concentrated in vacuo to give 173 (13 mg, 56%) as a gray solid: mp 245° C. (dec.); MALDIFT-HRMS m/z 233.0929 (M+H⁺, C₁₂H₁₃N₂O₃ requires 233.0921).

5-Butyrylaminoindole-2-carboxylic acid (174): Ethyl 5-butyrylaminoindole-2-carboxylate crystallized as a gray solid from EtOAc/ⁱPr₂O (84 mg, 110 mg theoretical, 76%): mp 210° C.; MALDIFT-HRMS m/z 275.1381 (M+H⁺, C₁₅H₁₉N₂O₃ requires 275.1390).

Ethyl 5-butyrylaminoindole-2-carboxylic acid was obtained by hydrolysis as described for 173 to give 174 (20 mg, theoretical 24.6 mg, 81%) as a gray solid: mp 239° C. (dec.); MALDIFT-HRMS m/z 247.1085 (M+H⁺, C₁₃H₁₅N₂O₃ requires 247.1077).

5-(N-Acetyl-N-methylamino)indole-2-carboxylic acid (176): A solution of ethyl 5-acetylaminoindole-2-carboxylate (Murakami, Y.; et al. Bull. Chem. Soc. Jpn. 1995, 43, 1281-1286) 169 (49 mg, 0.2 mmol), Boc₂O (52 mg, 0.24 mmol) and DMAP (24 mg, 0.2 mmol) in CH₂Cl₂ (4 mL) was stirred 1 h at 25° C., then quenched with the addition of 0.2 M HCl saturated with NaCl (10 mL). The reaction mixture was extracted with CH₂Cl₂ (2×20 mL), the combined organic layers were dried (Na₂SO₄), and concentrated in vacuo. Flash chromatography (1.5×25 cm, SiO₂, 50% EtOAc/hexane) afforded ethyl 5-acetylamino-N-(tert-butyloxycarbonyl)indole-2-carboxylate 175 (43 mg, 62%) as a beige solid: mp 122-123° C.; MALDIFT-HRMS m/z 347.1613 (M+H⁺, C₁₈H₂₃N₂O₃ requires 347.1601).

A solution of ethyl 5-acetylamino-N-(tert-butyloxycarbonyl)indole-2-carboxylate 175 (34.6 mg, 0.1 mmol) in DMF (0.4 mL) was treated with NaH (60%, 4.4 mg, 0.11 mmol) for 15 min at 0° C. and then MeI (21.3 mg, 0.15 mmol) was added. After 30 min stirring at 0° C., the reaction mixture was stirred for 15 h at 25° C., then quenched with the addition of saturated NaCl (3 mL). The reaction mixture was extracted with EtOAc (2×15 mL), the combined organic layers were dried (Na₂SO₄), and concentrated in vacuo. Flash chromatography (1.5×20 cm, SiO₂, 50% EtOAc/hexane) afforded ethyl 5-(N-acetyl-N-methylamino)-N-(tert-butyloxycarbonyl)indole-2-carboxylate 176 (31.5 mg, 88%) as a pale yellow oil: IR (film) vₘₐₓ 2979, 2925, 1730, 1660, 1372, 1341, 1321, 1260, 1230, 1190, 1159 cm⁻¹; MALDIFT-HRMS m/z 361.1756 (M+H⁺, C₁₉H₂₅N₂O₃ requires 361.1758). Ethyl 5-(N-acetyl-N-methylamino)-N-(tert-butyloxycarbonyl)indole-2-carboxylate 176 (31 mg, 0.086 mmol) was hydrolyzed following the procedure described for 173 to give 177 (15 mg, 75%) as a beige solid crystallized from (THF/hexane): mp 280° C. (dec.); MALDIFT-HRMS m/z 233.0928 (M+H⁺, C₁₂H₁₃N₂O₃ requires 233.0921).

5-(N-Acetyl-N-ethylamino)indole-2-carboxylic acid (179): A solution of ethyl 5-aminoindole carboxylate (200 mg, 0.98 mmol) in 16% HCl (2 mL) at 0° C. was treated with NaNO₂ (118 mg, 1.7 mmol) dissolved in H₂O (0.5 mL) and stirred for 30 min. The resulting brown mixture was added to 70% EtNH₂ (1.5 mL) at 0° C., stirred for 30 min at 0° C., and then 1 hr at ambient temperature. The mixture was quenched with 1 N NaOH (10 mL), and a dark red solid was collected by filtration. This solid was suspended in pyridine (5 mL) at 0° C. and treated with Ac₂O (110 mg, 1.1 mmol, 0.10 mL). The solution was warmed to ambient temperature and stirred for 18 hrs. The solution was then treated with 1 N HCl (5 mL) and extracted with EtOAc (3×30 mL). The combined organic layers were washed with 1 N HCl (3×20 mL), dried (Na₂SO₄), and concentrated in vacuo. Flash chromatography (1.5×25 cm, SiO₂, 25% EtOAc/hexane) afforded ethyl 5-(N-acetyl-N-ethylamino)indole-2-carboxylate 178 (160 mg, 60%) as an orange solid: mp 149-150° C.; MALDIFT-HRMS m/z 275.1392 (M+H⁺, C₁₅H₁₈N₂O₃ requires 275.1390).

A solution of ethyl 5-(N-acetyl-N-ethylamino)indole-2-carboxylate (63 mg, 0.23 mmol) in 2.3 mL of of EtOH was treated with 2.5 M Cs₂CO₃ (0.27 mL) for 2.5 h at 80° C., then diluted with H₂O and extracted with CH₂Cl₂ (15 mL). The aqueous layer was acidified with 1 M HCl (1 mL) and extracted with EtOAc (3×15 mL). The combined organic layers were dried (Na₂SO₄), and concentrated in vacuo. Flash chromatography (1.5×25 cm, SiO₂, 50% EtOAc/hexane with 1% acetic acid) afforded ethyl 5-(N-acetyl-N-ethylamino)indole-2-carboxylic acid (30 mg, 53%) as an orange solid: mp 203-204° C. (dec.); MALDIFT-HRMS m/z 247.1079 (M+H⁺, C₁₃H₁₄N₂O₃ requires 247.1077).

5- and 7-Formylindole-2-carboxylic acid (145 and 181): 120 was synthesized according to the procedure of Marx and Breitmaier (Marx, T.; Breitmaier, E. Liebigs Ann. Chem. 1992, 3, 183-186). 2 M BuLi in hexane (15.35 ml, 30.7 mmol) was added to 2-(3-bromophenyl)-[1,3]dioxolane 120 (7.00 g, 30.7 mmol) in 45 mL THF over 30 min at −78° C. The reaction was stirred for 30 at this temperature, and then N-formylpiperidine (3.82 g, 33.8 mmol) in THF (3 mL) was added over 15 min at −78° C. and the reaction mixture was allowed to warm over 6 h to 25° C., and then quenched with 3 M HCl (45 mL) and extracted with Et₂O (2×200 mL). The combined organic layers were dried (Na₂SO₄), and concentrated in vacuo. Flash chromatography (5×15 cm, SiO₂, 9% EtOAc/hexane) afforded 128 (5.3 g, 97%) as a colorless oil: ¹H NMR (CDCl₃, 500 MHz) d 10.04 (1H, s), 8.01 (1H, t, J=1.8 Hz), 7.90 (1H, dt, J=7.6, 1.5 Hz), 7.75 (1H, dt, J=7.7, 1.5 Hz), 7.56 (1H, t, J=7.6 Hz), 5.88 (1H, s); 4.05-4.17 (4H, m).

128 (3.54 g, 20 mmol) and methyl azidoacetate (6.91 g, 60 mmol) in MeOH (20 mL) were treated at −5° C. with a NaOMe solution generated in situ with Na (1.38 g, 60 mmol) and 20 mL of MeOH. The reaction mixture was stirred for 2 h at −5° C. and allowed to warm over 2 h to 25° C., then quenched with the addition of ice water (100 mL) and extracted with Et₂O (150 mL) and EtOAc (3×150 mL). The combined organic layers were washed with saturated NaCl (50 mL), dried (Na₂SO₄), and concentrated in vacuo. Flash chromatography (3×15 cm, SiO₂, 9% to 17% EtOAc/hexane) afforded methyl 2-azido-3-(3-[1,3]dioxolan-2-ylphenyl)acrylate (2.9 g, 53%) as a yellow oil: ¹H NMR (CDCl₃, 500 MHz) d 7.91 (1H, m), 7.83 (1H, dt, J=7.6, 1.5 Hz), 7.40 (1H, t, J=7.6 Hz), 6.92 (1H, s), 5.83 (1H, s); 3.92-4.17 (4H, m), 3.90 (3H, s).

Methyl 2-azido-3-(3-[1,3]dioxolan-2-ylphenyl)acrylate (2.9 g, 10.6 mmol) in m xylene (40 mL) was stirred for 3 h at 135° C., then concentrated in vacuo. Flash chromatography (3×20 cm, SiO₂, 6% to 17% EtOAc/hexane) afforded methyl 5-[1,3]dioxolan-2-ylindole-2-carboxylate 148 (1.10 g, 42%) as a white solid: mp 140-141° C.; ESI (positive) m/z 270 (M+Na⁺, C₁₃H₁₃NNaO₄), 248 (M+H⁺, C₁₃H₁₄NO₄); ESI (negative) m/z 282 (M+Cl⁻, C₁₃H₁₃ClNO₄), 246 (M−H⁻, C₁₃H₁₂NO₄); and methyl 7-[1,3]dioxolan-2-ylindole-2-carboxylate 180 (863 mg, 33%) as a white solid: mp 71-72° C.; ESI (positive) m/z 270 (M+Na⁺, C₁₃H₁₃NNaO₄), 248 (M+H⁺, C₁₃H₁₄NO₄); ESI (negative) m/z 282 (M+Cl⁻, C₁₃H₁₃ClNO₄), 246 (M−H⁻, C₁₃H₁₂NO₄).

148 (720 mg, 2.92 mmol) in dioxane/H₂O (4:1, 7.5 mL) was treated with 4 M LiOH (2.92 mL, 11.7 mmol) for 6 h at 25° C., then quenched with the addition of 1 M HCl (20 mL), and after being stirred 10 min, extracted with EtOAc (4×100 mL). The combined organic layers were dried (Na₂SO₄), and concentrated in vacuo. Flash chromatography (3×15 cm, SiO₂, 10% MeOH/CH₂Cl₂) afforded 181 (465 mg, 84%) as a white solid: mp 260° C. (dec).; IR (film) vₘₐₓ 3296, 2922, 1713, 1674, 1616, 1575, 1539, 1249, 1163 cm⁻¹.

180 (380 mg, 1.54 mmol) in dioxane/H₂O (4:1, 6 mL) was treated with 4 M LiOH (1.54 mL, 6.2 mmol) for 24 h at 25° C., then quenched with the addition of 1 M HCl (10 mL), and after being stirred for 10 min, extracted with EtOAc (4×60 mL). The combined organic layers were dried (Na₂SO₄), and concentrated in vacuo. Crystallization from THF/hexane afforded 145 (247 mg, 85%) as a white solid: mp 280° C. (dec); IR (film) vₘₐₓ 3283, 1707, 1672, 1613, 1573, 1536, 1311, 1250, 1232, 1212, 1163, 1100, 814, 769, 742 cm⁻¹.

5-Acetylindole-2-carboxylic acid (183): Ethyl 5-acetylindole-2-carboxylate (23 mg, 0.1 mmol) was hydrolyzed following the procedure described for 112-114 to give 183 (20 mg, 99%) as a white solid: mp 305° C. (dec); ¹H NMR (DMSO-d₆, 500 MHz) δ 12.12 (1H, brs), 8.41 (1H, d, J=1.5 Hz), 7.85 (1H, dd, J=8.8, 1.5 Hz), 7.49 (1H, d, J=8.8 Hz), 7.26-7.27 (1H, m), 2.60 (3H, s).

7-Acetylindole-2-carboxylic acid (184): Ethyl 7-acetylindole-2-carboxylic acid (23 mg, 0.1 mmol) was hydrolyzed following the procedure described for 112-114 to give 183 (17.5 mg, 86%) as a yellow solid: mp 225-226° C. (dec); MALDIFT-HRMS m/z 204.0657 (M+H⁺, C₁₁H₁₀NO₃ requires 204.0655).

5-Propionylindole-2-carboxylic acid (185): Ethyl 5-propionylindole-2-carboxylate (49.1 mg, 0.2 mmol) was hydrolyzed following the procedure described for 112-114 to give 185 (43 mg, 99%) as a white solid: mp 270° C. (dec); MALDIFT-HRMS m/z 218.0817 (M+H⁺, C₁₂H₁₂NO₃ requires 218.0812).

5-Butyrylindole-2-carboxylic acid (186): Ethyl 5-butyrylindole-2-carboxylate (51.9 mg, 0.2 mmol) was hydrolyzed following the procedure described for 112-114 to give 186 (43 mg, 99%) as a white solid: mp 260° C. (dec); MALDIFT-HRMS m/z 232.0974 (M+H⁺, C₁₃H₁₄NO₃ requires 232.0968).

5-Methoxycarbonylindole-2-carboxylic acid (92): 181 (425 mg, 2.25 mmol) and benzyl alcohol (1.46 g, 13.5 mmol) in 10 mL of CH₂Cl₂ were treated with EDCl (431 mg, 2.25 mmol) and DMAP (55 mg, 0.5 mmol) at 0° C. The reaction mixture was stirred for 1 h at 0° C. and 1 h at 25° C., then quenched with 1 M HCl (10 mL), and extracted with CH₂Cl₂ (2×30 mL). The combined organic layers were dried (Na₂SO₄), and concentrated in vacuo. Flash chromatography (2.5×20 cm, SiO₂, 6% EtOAc/hexane) afforded benzyl 5-formylindole-2-carboxylate (308 mg, 84%) as a white solid: mp 172° C.; MALDIFT-HRMS m/z 280.0963 (M+H⁺, C₁₇H₁₄NO₃ requires 280.0968).

Benzyl 5-formylindole-2-carboxylate (148 mg, 0.53 mmol) and PDC (538 mg, 1.43 mmol) in 1 mL DMF were stirred for 5 days at 25° C., then quenched with 1 M HCl (20 mL) and extracted with EtOAc (3×50 mL). The combined organic layers were washed with saturated NaCl (20 mL), dried (Na₂SO₄), and concentrated in vacuo. Flash chromatography (2.5×20 cm, SiO₂, 5% MeOH/CH₂Cl₂) afforded 191 (78 mg, 50%) as a white solid: mp 244° C. (dec.); MALDIFT-HRMS m/z 278.0812 (M+H—H₂O+, C₁₇H₁₂NO₃ requires 278.0817).

191 (12.8 mg, 0.043 mmol) in 1 mL THF was treated with 0.4 M CH₂N₂ in Et₂O (110 μL, 0.06 mmol). The reaction mixture was stirred for 5 min at 25° C., and then evaporated. Flash chromatography (0.5×5 cm, SiO₂, 17% EtOAc/hexane) afforded benzyl 5-methoxycarbonyl-indole-2-carboxylate (9.9 mg, 74%) as a white solid: mp 128-129° C.; MALDIFT-HRMS m/z 310.1080 (M+H⁺, C₁₈H₁₆NO₄ requires 310.1074).

Benzyl 5-methoxycarbonylindole-2-carboxylate (9.8 mg, 0.032 mmol) and 10% Pd/C (2.5 mg) in 640 μL THF were stirred under H₂ (1 bar) for 1 h at 25° C., then filtered over Celite. The catalyst was washed with THF (1 mL) and the filtrate was evaporated to afford 5-methoxycarbonyl-indole-2-carboxylic acid 192 (6.3 mg, 91%) as an off-white solid: mp 281° C. (dec.); ESI (positive) m/z 220 (M+H⁺, C₁₁H₁₀NO₄); ESI (negative) m/z 218 (M−H⁻, C₁₁H₈NO₄).

7-Methoxycarbonylindole-2-carboxylic acid (190): 145 (33 mg, 0.17 mmol) and benzyl alcohol (75 mg, 0.7 mmol) in 680 μL of CH₂Cl₂ were treated with EDCl (33 mg, 0.17 mmol) and DMAP (4 mg, 0.035 mmol) at 0° C. The reaction mixture was stirred for 1 h at 0° C. and 1 h at 25° C., then quenched with 1 M HCl (5 mL), and extracted with CH₂Cl₂ (2×10 mL). The combined organic layers were dried (Na₂SO₄), and concentrated in vacuo. Flash chromatography (1.5×25 cm, SiO₂, 6% EtOAc/hexane) afforded benzyl 7-formylindole-2-carboxylate (35.6 mg, 73%) as a white solid: mp 62° C.; MALDIFT-HRMS m/z 302.0794 (M+Na⁺, C₁₇H₁₃NNaO₃ requires 302.0788).

Benzyl 7-formylindole-2-carboxylate (28 mg, 0.1 mmol) and PDC (100 mg, 0.27 mmol) in 200 μL DMF were stirred for 4 days at 25° C., then quenched with 1 M HCl (5 mL) and extracted with EtOAc (3×15 mL). The combined organic layers were washed with saturated NaCl (10 mL), dried (Na₂SO₄), and concentrated in vacuo. Flash chromatography (1.5×25 cm, SiO₂, 5% MeOH/CH₂Cl₂) afforded 189 (18.6 mg, 63%) as a white solid: IR (film) vₘₐₓ 3450, 1712, 1672, 1281, 1236, 1200, 1149, 753, 734 cm⁻¹; MALDIFT-HRMS m/z 296.0906 (M+H⁺, C₁₇H₁₄NO₄ requires 296.0917).

189 (17.5 mg, 0.059 mmol) in 1 mL THF was treated with 0.4 M CH₂N₂ in Et₂O (150 μL, 0.06 mmol). The reaction mixture was stirred for 5 min at 25° C., and then evaporated. Flash chromatography (0.5×5 cm, SiO₂, 17% EtOAc/hexane) afforded benzyl 7-methoxycarbonyl-indole-2-carboxylate (15 mg, 82%) as a white solid: IR (film) vₘₐₓ 3450, 2952, 1714, 1699, 1306, 1281, 1229, 1203, 1141, 752 cm⁻¹; MALDIFT-HRMS m/z 310.1074 (M+H⁺, C₁₈H₁₆NO₄ requires 310.1074).

Benzyl 7-methoxycarbonylindole-2-carboxylate (10 mg, 0.032 mmol) and 10% Pd/C (2.5 mg) in 640 μL THF were stirred under H₂ (1 bar) for 1 h at 25° C., then filtered over Celite. The catalyst was washed with THF (1 mL) and the filtrate was evaporated to afford 7-methoxycarbonyl-indole-2-carboxylic acid 190 (7 mg, quant.) as an off-white solid: mp 216° C. (dec.); MALDIFT-HRMS m/z 220.0610 (M+H⁺, C₁₁H₁₀NO₄ requires 220.0610).

5-Ethoxycarbonylindole-2-carboxylic acid (193): 191 (17.7 mg, 0.06 mmol) and 1 μL DMF in 3 mL of CH₂Cl₂ were treated with oxalyl chloride (11.4 mg, 0.09 mmol) at 0° C. The reaction mixture was allowed to warm over 40 min to 25° C., then cooled to 0° C., and 1 mL EtOH was then added. The reaction mixture was allowed to warm over 1 h to 25° C., and was then evaporated. Flash chromatography (1.5×20 cm, SiO₂, 9% EtOAc/hexane) afforded benzyl 5-ethoxycarbonylindole-2-carboxylate (14.7 mg, 76%) as a white solid: mp 123° C.; MALDIFT-HRMS m/z 324.1220 (M+H⁺, C₁₉H₁₈NO₄ requires 324.1230).

Benzyl 5-ethoxycarbonylindole-2-carboxylate (12 mg, 0.037 mmol) and 10% Pd/C (3 mg) in 740 μL THF were stirred under H₂ (1 bar) for 1 h at 25° C., then filtered over Celite. The catalyst was washed with THF (1 mL) and the filtrate was evaporated. Crystallization from THF/hexane afforded 193 (6.0 mg, 69%) as a white solid: mp 275° C. dec.; MALDIFT-HRMS m/z 234.0760 (M+H⁺, C₁₂H₁₂NO₄ requires 234.0761).

5-Methylcarbamoylindole-2-carboxylic acid (194): 191 (17.7 mg, 0.06 mmol) and 1 μL DMF in 3 mL of CH₂Cl₂ were treated with oxalyl chloride (11.4 mg, 0.09 mmol) at 0° C. The reaction mixture was allowed to warm over 40 min to 25° C., then cooled to 0° C., and 300 μL 40% MeNH₂ was added. The reaction mixture was stirred 10 min at 0° C., then quenched with 1 M HCl (5 mL), and extracted with CH₂Cl₂ (3×40 mL). The combined organic layers were dried (Na₂SO₄), and concentrated in vacuo to afford benzyl 5-methylcarbamoylindole-2-carboxylate (16.9 mg, 91%): mp 197° C.; MALDIFT-HRMS m/z 309.1235 (M+H⁺, C₁₈H₁₇N₂O₃ requires 309.1234).

Benzyl 5-methylcarbamoylindole-2-carboxylate (13.5 mg, 0.044 mmol) and 10% Pd/C (3 mg) in 440 μL THF were stirred under H₂ (1 bar) for 1 h at 25° C., then filtered over Celite. The catalyst was washed with THF (1 mL) and the filtrate was evaporated to afford 194 (9.6 mg, quant.) as a white solid: mp 281° C. (dec.); MALDIFT-HRMS m/z 219.0764 (M+H⁺, C₁₁H₁₁N₂O₃ requires 219.0764).

5-Dimethylcarbamoylindole-2-carboxylic acid (195): Using the same procedure as for 194, but instead quenching the acid chloride with 40% HNMe₂ (350 μL), afforded benzyl 5-dimethyl-carbamoylindole-2-carboxylate (18 mg, 93%) as an off-white solid: mp 123° C. (dec.); MALDIFT-HRMS m/z 323.1383 (M+H⁺, C₁₉H₁₉N₂O₃ requires 323.1390); which was deprotected to afford 195 (12.7 mg, 98%) as a white solid: mp 217° C. (dec.); MALDIFT-HRMS m/z 233.0921 (M+H⁺, C₁₂H₁₃N₂O₃ requires 233.0921).

5-Carbamoylindole-2-carboxylic acid (196): Ethyl 5-cyanoindole-2-carboxylate 139 (124 mg, 0.58 mmol) in 900 μL of DMSO was treated at 0° C. with K₂CO₃ (12.5 mg, 0.09 mmol) and 30% H₂O₂ (180 μL). The reaction mixture was allowed to warm over 1 h to 25° C., then diluted with H₂O (10 mL) and extracted with EtOAc (2×30 mL). The combined organic layers were washed with saturated NaCl (10 mL), dried (Na₂SO₄), and concentrated in vacuo. Flash chromatography (2.5×20 cm, SiO₂, 33% hexane/EtOAc) afforded ethyl 5-carbamoylindole-2-carboxylate as a white solid: mp 210° C.; MALDIFT-HRMS m/z 233.0922 (M+H⁺, C₁₂H₁₃N₂O₃ requires 233.0921).

A solution of ethyl 5-carbamoylindole-2-carboxylate (35 mg, 0.15 mmol) in 675 μL of EtOH was treated with 2 M Cs₂CO₃ (225 μL, 0.45 mmol) for 2.5 h at 80° C. and then was acidified with 1 M HCl (3 mL) and H₂O (10 mL), and extracted with 1/1 THF/EtOAc (2×100 mL). The combined organic layers were dried (Na₂SO₄), and concentrated in vacuo. Crystallization from THF/CHCl₃ afforded 196 (23 mg, 68%, 2 steps) as an off-white solid: mp 260° C. (dec.); MALDIFT-HRMS m/z 205.0615 (M+H⁺, C₁₀H₉N₂O₃ requires 205.0608).

1,2-Dihydropyrrolo[3,2-e]benzofuran-7-carboxylic acid (199): 197 (41 mg, 0.28 mmol) and methyl azidoacetate (92 mg, 1.1 mmol) in MeOH (0.3 mL) were treated at 0° C. with sodium methoxide solution generated in situ with Na (25 mg, 1.1 mmol) and 0.3 mL of MeOH. The reaction mixture was allowed to warm over 2 h to 25° C., then quenched with the addition of ice water (5 mL) and extracted with EtOAc (2×50 mL). The combined organic layers were dried (Na₂SO₄), and concentrated in vacuo, which afforded the azido ester (33 mg, 49%) as an off-white solid. IR (film) vₘₐₓ 2117, 1713, 1616, 1450, 1435, 1260, 1237, 1090, 989, 778 cm⁻¹.

The azido ester (33 mg, 0.14 mmol) in m-xylene (2 mL) was stirred for 3 h at 130° C., then concentrated in vacuo. Flash chromatography (2.5×20 cm, SiO₂, 9% EtOAc/hexane) afforded 198 (9.6 mg, 33%) as a yellow solid: IR (film) vₘₐₓ 3311, 2919, 1682, 1527, 1442, 1330, 1263, 1218, 1041, 969, 764 cm⁻¹; MALDIFT-HRMS m/z 217.0729 (M⁺, C₁₂H₁₁NO₃ requires 217.0733).

198 (9.6 mg, 0.044 mmol) was dissolved in 1.5 mL of 1:1:1 THF:MeOH:H₂O. LiOH (7.4 mg, 0.18 mmol) was added at room temperature and the solution was stirred 14 h. The reaction mixture was then quenched with 1 M HCl and extracted with diethyl ether (4×10 mL). The combined organic layers were dried over Na₂SO₄ and concentrated in vacuo. The crude acid 199 was used without further purification. MALDIFT-HRMS m/z 203.0576 (M⁺, C₁₁H₉NO₅ requires 203.0577).

Pyrrolo[3,2-e]benzofuran-7-carboxylic acid (202): 200 (40 mg, 0.27 mmol) and methyl azidoacetate (92 mg, 1.1 mmol) in MeOH (0.3 mL) were treated at 0° C. with sodium methoxide solution generated in situ with Na (25 mg, 1.1 mmol) and 0.3 mL of MeOH. The reaction mixture was allowed to warm over 2 h to 25° C., then quenched with the addition of ice water (5 mL) and extracted with EtOAc (2×50 mL). The combined organic layers were dried (Na₂SO₄), and concentrated in vacuo to afford the azido etser (28 mg, 43%) as an off-white solid. ¹H NMR (CDCl₃, 400 MHz) δ 8.14 (1H, d, J=7.6 Hz), 7.70 (1H, d, J=2.1 Hz), 7.50 (1H, d, J=8.2 Hz), 7.36 (1H, t, J=7.9 Hz), 7.26 (1H, d, J=7.9 Hz), 6.96 (1H, d, J=1.5 Hz), 3.97 (3H, s).

The azido ester (28 mg, 0.12 mmol) in m-xylene (2 mL) was stirred for 3 h at 130° C., then concentrated in vacuo. Flash chromatography (2.5×20 cm, SiO₂, 9% EtOAc/hexane) afforded 201 (13 mg, 22%) as a beige solid: mp 154-155° C.; MALDIFT-HRMS m/z 215.0586 (M⁺, C₁₂H₉NO₃ requires 215.0582).

201 (13 mg, 0.060 mmol) was dissolved in 1.5 mL of 2:1:1 THF:MeOH:H₂O. LiOH (7.4 mg, 0.18 mmol) was added at room temperature and the HCl and extracted with diethyl ether (4×10 mL). The combined organic layers were dried over Na₂SO₄ and concentrated in vacuo to afford 202 (6.0 mg, 49%) as a beige solid: mp 183-184° C.; ESI-MS m/z 200 (M−H⁻, C₁₁H₁₇NO₃ requires 200.0353).

6-Oxo-cyclopenten[e]indole-2-carboxylic acid (207). Ozonolysis (Zheng, Y.-J; Merz, Jr., K. M. J. Am. Chem. Soc. 1992, 114, 10498-10507) of the methyl enolate of b-tetralone (Guspanová, L.; et al. Helv. Chim. Acta 1997, 80, 1375) (900 mg, 5.6 mmol) provided methyl 3-(2-formylpenyl)propionate 204 (570 mg, 53%) as a colorless oil: ¹H NMR (CDCl₃, 500 MHz) δ 10.21 (1H, s), 7.81 (1H, dd, J=7.5, 1.3 Hz), 7.52 (1H, td, J=7.5, 1.6 Hz), 7.42 (1H, td, J=7.5, 1.1 Hz), 7.33 (1H, dm, J=8.1 Hz), 3.36 (2H, t, J=7.7 Hz), 2.66 (2H, t, J=7.7 Hz).

204 (450 mg, 2.34 mmol) and methyl azidoacetate (1.2 g, 10.4 mmol) in MeOH (3 mL) were treated at 0° C. with a sodium methoxide solution generated in situ with Na (239 mg, 10.4 mmol) and 4 mL of MeOH. The reaction mixture was allowed to warm over 2 h to 25° C., then quenched with the addition of ice water (15 mL) and extracted with EtOAc (2×150 mL). The combined organic layers were dried (Na₂SO₄), and concentrated in vacuo. Flash chromatography (2×20 cm, SiO₂, 6% to 17% EtOAc/hexane) afforded methyl 2-azido-3-[2-(2-methoxycarbonyl)ethylphenyl]acrylate (268 mg, 40%) as a pale yellow oil: ¹H NMR (CDCl₃, 500 MHz) δ 7.89-7.91 (m, 1H), 7.22-7.28 (m, 3H), 7.15 (s, 1H), 3.93 (s, 3H), 3.68 (s, 3H), 3.01 (t, J=7.9 Hz), 2.58 (t, J=8.1 Hz).

Methyl 2-azido-3-[2-(2-methoxycarbonyl)ethylphenyl]acrylate (264 mg, 0.91 mmol) in m-xylene (18 mL) was stirred for 3 h at 130° C., then concentrated in vacuo. Flash chromatography (2×30 cm, SiO₂, 9% to 20% EtOAc/hexane) afforded 205 (185 mg, 78%) as an off-white solid: mp 70° C.; MALDIFT-HRMS m/z 284.0888 (M+Na⁺, C₁₄H₁₅NNaO₄ requires 284.0893).

205 (130 mg, 0.5 mmol) in 4 mL of THF and 4 mL of MeOH was treated at 0° C. with NaOH (22 mg, 0.55 mmol) in 1 mL H₂O. The reaction mixture was allowed to warm over 24 h to 25° C., then extracted with EtOAc (3×50 mL). The combined organic layers were dried (Na₂SO₄), and concentrated in vacuo. Flash chromatography (3×10 cm, SiO₂, 50% to 0% hexane/EtOAc then 10% MeOH/CH₂Cl₂) afforded 3-(2-methoxycarbonyl)indol-4-ylpropionic acid (78 mg, 63%) as an off-white solid: mp 178° C.; MALDIFT-HRMS m/z 247.0839 (M⁺, C₁₃H₁₃NO₄ requires 247.0839).

3-(2-Methoxycarbonyl)indol-4-ylpropionic acid (45 mg, 0.18 mmol) and 1 μL of DMF in 2 mL of CH₂Cl₂ at 0° C. was treated with oxalyl chloride (35 mg, 0.27 mmol). The reaction mixture was allowed to warm over 1 h to 25° C., then concentrated in vacuo. The residue was dissolved in 9 mL of 1,2-dichloroethane and was treated with AlCl₃ (24.3 mg, 0.18 mmol) at 0° C. The reaction mixture was allowed to warm over 2 h to 25° C., then quenched with the addition of ice water (10 mL), and extracted with CH₂Cl₂ (3×30 mL). The combined organic layers were dried (Na₂SO₄), and concentrated in vacuo. Flash chromatography (2.5×15 cm, SiO₂, 33% EtOAc/hexane then 10% MeOH/CH₂Cl₂) afforded starting material (36 mg, 80% recovery) and 206 (7.2 mg, 17%, 86% based on recovered s.m.) as a white solid: mp 243° C. (dec.); MALDIFT-HRMS m/z 230.0811 (M+H⁺, C₁₃H₁₂NO₃ requires 230.0812).

206 (10 mg, 0.044 mmol) in dioxane/H₂O (4:1, 250 μL) was treated with 4 M LiOH (44 μL, 0.176 mmol) for 14 h at 25° C., then quenched with the addition of 1 M HCl (5 mL), and extracted with EtOAc/THF (2:1, 3×30 mL). The combined organic layers were dried (Na₂SO₄), and concentrated in vacuo to afford 207 (9.2 mg, 98%) as an off-white solid: mp 320° C. (dec.); MALDIFT-HRMS m/z 216.0654 (M+H⁺, C₁₂H₁₀NO₃ requires 216.0655).

6-Oxo-cyclopenten[f]indole-2-carboxylic acid (212): 4-Formylcinnamic acid 208 (881 mg, 5 mmol) in 20 mL of THF was treated dropwise over 10 min with 0.4 M CH₂N₂ in Et₂O (12.5 mL). The reaction mixture was quenched with AcOH (1 drop) and concentrated in vacuo. Filtration through a plug of silica gel (CH₂Cl₂/hexane) afforded methyl 4-formylcinamate (741 mg, 78%) as an oil: ¹H NMR (CDCl₃, 500 MHz) δ 10.03 (1H, s), 7.90 (2H, dm, J=8.3 Hz), 7.72 (1H, d, J=16.7 Hz), 7.67 (2H, dm, J=8.4 Hz), 6.55 (1H, d, J=16.1 Hz), 3.83 (3H, s).

Methyl 4-formylcinamate (493 mg, 2.6 mmol) and PtO₂ (35 mg, 0.16 mmol) in 30 mL of EtOAc were strirred for 40 min at 25° C. under H₂ (1 bar), then filtered over Celite. The catalyst was washed with EtOAc (5 mL), and the filtrate was concentrated in vacuo. Flash chromatography (1.5×15 cm, SiO₂, 4% EtOAc/hexane) afforded methyl-3-(4-formylphenyl)propanoate 209 (318 mg, 64%) as a colorless oil: ¹H NMR (CDCl₃, 500 MHz) δ 9.99 (1H, s), 7.82 (2H, d, J=7.3 Hz), 7.38 (2H, d, J=7.7 Hz), 3.68 (3H, s), 3.04 (2H, t, J=7.7 Hz), 2.68 (2H, t, J=7.7 Hz).

209 (312 mg, 1.62 mmol) and methyl azidoacetate (747 mg, 6.5 mmol) in MeOH (3 mL) were treated at 0° C. with sodium methoxide solution generated in situ with Na (149 mg, 6.5 mmol) and 3 mL of MeOH. The reaction mixture was allowed to warm over 2 h to 25° C., then quenched with the addition of ice water (15 mL) and extracted with EtOAc (2×150 mL). The combined organic layers were dried (Na₂SO₄), and concentrated in vacuo. Flash chromatography (2×20 cm, SiO₂, 17% EtOAc/hexane) afforded methyl 2-azido-3-[3-(2-methoxycarbonyl)ethylphenyl]acrylate (347 mg, 74%) as a pale yellow oil.

Methyl 2-azido-3-[3-(2-methoxycarbonyl)ethylphenyl]acrylate (160 mg, 0.55 mmol) in m-xylene (10 mL) was stirred for 3 h at 130° C., then concentrated in vacuo. Flash chromatography (2.5×20 cm, SiO₂, 9% EtOAc/hexane) afforded 210 (87 mg, 60%) as a white solid: mp 71-72° C.; MALDIFT-HRMS m/z 261.0996 (M⁺, C₁₄H₁₅NO₄ requires 261.0996).

210 (150 mg, 0.57 mmol) in 2.5 mL of THF and 2.5 mL of MeOH was treated at 0° C. with NaOH (23 mg, 0.57 mmol) in 1 mL H₂O. The reaction mixture was allowed to warm over 15 h to 25° C., then extracted with EtOAc (3×50 mL). The combined organic layers were dried (Na₂SO₄), and concentrated in vacuo. Flash chromatography (3×10 cm, SiO₂, 33% to 100% EtOAc/hexane then 10% MeOH/CH₂Cl₂) afforded 3-(2-methoxycarbonyl)indol-6-ylpropionic acid (83 mg, 58%) as a white solid: mp 158-159° C.; MALDIFT-HRMS m/z 270.0732 (M+Na⁺, C₁₃H₁₃NNaO₄ requires 270.0737).

3-(2-Methoxycarbonyl)indol-6-ylpropionic acid (80 mg, 0.32 mmol) and 1 μL of DMF in 4 mL of CH₂Cl₂ at 0° C. were treated with oxalyl chloride (62 mg, 0.49 mmol). The reaction mixture was allowed to warm over 1 h to 25° C., then concentrated in vacuo. The residue was dissolved in 10 mL of 1,2-dichloroethane and was treated with AlCl₃ (43 mg, 0.32 mmol) at 0° C. The reaction mixture was allowed to warm over 2 h at 25° C., then quenched with the addition of ice water (10 mL), and extracted with CH₂Cl₂ (3×50 mL). The combined organic layers were dried (Na₂SO₄), and concentrated in vacuo. Flash chromatography (2.5×15 cm, SiO₂, 25% EtOAc/hexane then 10% MeOH/CH₂Cl₂) afforded starting material (34 mg, 43% recovery), the undesired regioisomer (11.3 mg, 15%), and 211 (7.8 mg, 11%, 18% based on recovery of s.m.) as a white solid: mp 249° C.; MALDIFT-HRMS m/z 230.0809 (M+H⁺, C₁₃H₁₂NO₃ requires 230.0812).

211 (7 mg, 0.031 mmol) in dioxane/H₂O (4:1, 160 μL) was treated with 4 M LiOH (31 μL, 0.122 mmol) for 12 h at 25° C., then quenched with the addition of 1 M HCl (1 mL) and H₂O (2 mL), and extracted with EtOAc/THF (2:1, 4×30 mL). The combined organic layers were dried (Na₂SO₄), and concentrated in vacuo to afford x (6 mg, 91%) as an off-white solid: mp 230° C. (dec.); MALDIFT-HRMS m/z 216.0656 (M+H⁺, C₁₂H₁₀NO₃ requires 216.0655).

[1,3]Dioxolo[e]indole-2-carboxylic acid (215): The azido ester product of 213 (71.2 mg, 0.288 mmol) in p-xylene (5 mL) was stirred for 16 h at 130° C., then concentrated in vacuo. Flash chromatography (2.5×20 cm, SiO₂, 17% EtOAc/hexane) afforded 214 (17.0 mg, 63.1 mg theoretical, 27%) as a yellow solid: ¹H NMR (CDCl₃, 600 MHz) d: 8.78, (1H, br s), 7.02 (1H, s), 6.93 (1H, d, J=8.2 Hz), 6.82 (1H, d, J=8.2 Hz), 5.97 (2H, s), 3.87 (3H, s).

[1,3]Dioxolo[f]indole-2-carboxylic acid (218): Using the same procedure as for 213 (Rodrigues, J. A. R.; et al. Tetrahedron Lett. 1995, 36, 59-62), piperonal was treated with methyl azidoacetate to afford methyl 2-azido-3-benzo[1,3]dioxol-5-ylacrylate (235 mg, 48%) as a yellow solid. Methyl 2-azido-3-benzo[1,3]dioxol-5-ylacrylate (230 mg, 0.93 mmol) in m-xylene (5 mL) was stirred for 1 h at 135° C., then concentrated in vacuo. Flash chromatography (dry loaded, 1.5×20 cm, SiO₂, 17% EtOAc/hexane) afforded 217 (142 mg, 70%) as a pale yellow solid: ¹H NMR (acetone-d₆, 500 MHz) δ 10.77 (1H, br s), 7.05 (1H, dd, J=2.1, 0.9 Hz), 7.03 (1H, m), 6.95 (1H, m), 5.98 (2H, s), 3.84 (3H, s).

217 (44 mg, 0.2 mmol) in dioxane/H₂O (4:1, 1 mL) was treated with 4 M LiOH (200 μL) for 15 h at 25° C., then quenched with the addition of 1 M HCl (2 mL) and ice, and extracted with EtOAc (2×20 mL). The combined organic layers were dried (Na₂SO₄), and concentrated in vacuo. Flash chromatography (dry loaded, 1.5×20 cm, SiO₂, 5% to 10% MeOH/CH₂Cl₂) afforded 218 (30.5 mg, 74%) as a white solid: mp >300° C. (dec) (lit. (Patent: PCT, U.S. 1998, 25829) 250-251° C.); ¹H NMR (DMSO-d₆, 500 MHz) δ 12.43 (1H, br s), 11.55 (1H, s), 7.04 (1H, s), 6.93 (1H, m), 6.85 (1H, s), 5.97 (2H, s).

Acid 223 (x): indan 219 (2.5 g, 21.19 mmol, 1.0 equiv.) was dissolved in CH₂Cl₂ (25 mL, 0.9 M) and cooled to 0° C. TiCl₄ (28.17 mL, 1 M solution, 28.18 mmol, 1.33 equiv.) was added and the reaction stirred for 30 minutes. α,α-Dichloromethyl methyl ether (2.53 mL, 27.97 mmol, 1.32 equiv.) was added with additional cooling. The reaction was warmed to 23° C. for 1 h, then solvent was removed in vacuo. The residue was treated to a short column (SiO₂, 2×30 mL, 50% EtOAc/hexanes) to remove baseline materials, then subjected to preparative HPLC (5 mL neat injection, isocratic, 98:2 hexanes/EtOAc, desired fraction retention time=9.292 min). This afforded pure 4-indanaldehyde 220 (1.0 g, 3.0 g theoretical, 33%) as a brown oil: R_{f}=0.30 (hexanes); IR (film) vₘₐₓ 2961, 2926, 2844, 1685, 1456 cm⁻¹.

Methyl 2-azido-3-indan-4-yl-acrylate (33.5 mg, 0.1379 mmol, 1.0 equiv.) was dissolved in p-xylenes (2.75 mL, 0.05 M) and heated to 130° C. for 16 h. The mixture was cooled and the solvent removed. PTLC (SiO₂, 20 cm×20 cm, 9:1 EtOAc/hexanes) afforded 222 (7.7 mg, 29.6 mg theoretical, 26%) as a white solid and a minor isomer. Data for both isomers: R_{f}=0.11 (15:1 hexanes/EtOAc).

222 (7.7 mg, 0.0358 mmol, 1.0 equiv.) was dissolved in a 3:1:1 mixture of THF, MeOH, and H₂O (0.36 mL, 0.2 M). LiOH (4.1 mg, 0.0985 mmol, 2.75 equiv.) was added and the mixture was stirred for 2.5 h. The solvent removed and then the mixture was treated to PTLC (SiO₂, 20 cm×20 cm, 75% EtOAc/hexanes) which afforded 223 (7.1 mg, 7.2 mg theoretical, 99%) as a white solid: R_{f}=0.11 (15:1 hexanes/EtOAc).

Benz[e]indole-2-carboxylic acid (226): A solution of KOH (87%, 52 mg, 0.8 mmol) and ethyl benz[e]indole-2-carboxylate 225 (48 mg, 0.2 mmol) in 600 μL of EtOH was stirred for 30 min at 80° C., then quenched with the addition of 1 M HCl (1 mL) and ice, and extracted with EtOAc (2×20 mL). The combined organic layers were dried (Na₂SO₄), and concentrated in vacuo. Crystallization (THF/hexane) afforded 226 (28 mg, 70%) as a white solid: mp 245° C. (dec.) (lit. (Patent: WO 9109849) mp 246° C.).

Benz[f]indole-2-carboxylic acid (232): Ethyl benz[f]indole-2-carboxylate 231 (48 mg, 0.2 mmol) (mp 180-181° C. (lit. (Boger, D. L.; et al. J. Am. Chem. Soc. 2000, 122, 6382-6394) mp 188-190° C.)) was hydrolyzed according to the procedure described for x to afford x (32 mg, 80%) as a yellow solid: mp 280° C. (dec.); IR (film) vₘₐₓ 3418, 2960, 1678, 866, 738 cm⁻¹.

### DETAILED DESCRIPTION OF FIGURES

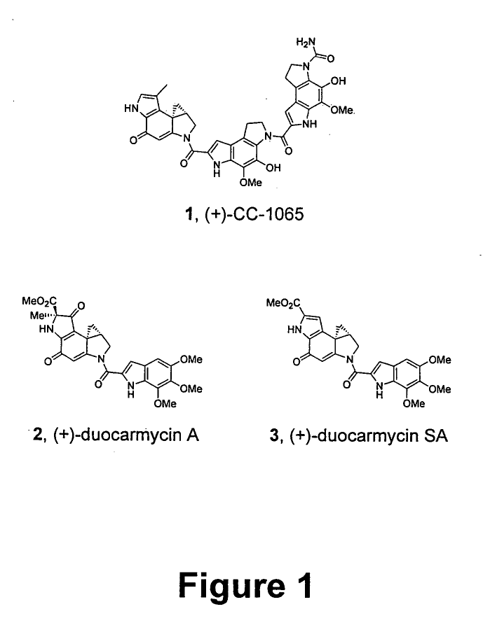

FIG. 1 shows the structures of CC-1065 (1), duocarmycin A (2), and duocarmycin SA (3) which constitute the parent members of a class of potent anti-tumor antibiotics that derive their properties through sequence-selective alkylation of duplex DNA.

FIG. 2 shows the front and groove view of the ¹H NMR derived solution structure of (+)-duocarmycin SA bound to a high affinity alkylation site within d(GACTAATTGAC)-d(GTCAATTAGTC) highlighting the minor groove embedded indole C5 methoxy group (Eis, P. S.; et al. J. Mol. Biol. 1997, 272, 237-252). Moreover, the C5 methoxy group of 3 is found deeply embedded in the minor groove with methyl group extending into, not away from, the minor groove floor, potentially benefiting from hydrophobic contacts.

FIG. 3 is a scheme that shows the preparation of the compounds 6B-78B. The non-commercially available indole-2-carboxylic acids were obtained from the corresponding azido cinnamates derived from condensation of substituted benzaldehydes with methyl α-azidoacetate. The azidocinnamates were subjected to the Hemetsberger reaction (Hemetsberger, H.; et al. Montash. Chem. 1969, 100, 1599-1603) followed by saponification of the esters to provide the substituted indole-2-carboxylic acids. Acid-catalyzed deprotection of seco-N-BOC-CBI (natural or unnatural enantiomer) followed by coupling of the resulting hydrochloride salt with the indole-2-carboxylic acids (3 equiv EDCl, DMF, 25° C., 14 h) in the absence of added base provided the seco agents (Boger, D. L.; et al. J. Am. Chem. Soc. 1989, 111, 6461-6463; Boger, D. L.; et al. J. Org. Chem. 1990, 55, 5823-5832; Boger, D. L.; Ishizaki, T. Tetrahedron Lett. 1990, 31, 793-796; Boger, D. L.; et al. Bioorg. Med. Chem. Lett. 1991, 1, 115-120). Spirocyclization was effected by treatment with DBU (Boger, D. L.; et al. J. Am. Chem. Soc. 1998, 120, 11554-11557; Boger, D. L.; et al. J. Org. Chem. 1999, 64, 5241-5244) or NaH (Boger, D. L.; et al. J. Am. Chem. Soc. 1989, 111, 6461-6463; Boger, D. L.; et al. J. Org. Chem. 1990, 55, 5823-5832; Boger, D. L.; Ishizaki, T. Tetrahedron Lett. 1990, 31, 793-796; Boger, D. L.; et al. Bioorg. Med. Chem. Lett. 1991, 1, 115-120) providing 6B-78B.

FIG. 4 is a table comparing the IC₅₀'s of the natural compounds to some analogs. The comparison compounds for examining the effects of the indole substituents of the CBI analogues of duocarmycin SA (3) are CBI-TMI (4) (Boger D. L.; Yun, W. J. Am. Chem. Soc. 1994, 116, 7996-8006) and CBI-indole (5, FIG. 4). The former contains the 5,6,7-trimethoxy substituents of duocarmycin SA (3) while the latter incorporates the parent unsubstituted indole. (+)-CBI-TMI (4) was found to be nearly 100-fold more potent than (+)-CBI-indole (5) with the 5,6,7-trimethoxyindole substitution increasing the L1210 cytotoxic potency 90 times. This effect of the 5,6,7-trimethoxy substitution is analogous, but more pronounced, than the 6-10 fold effect observed with duocarmycin SA (Boger, D. L.; et al. J. Am. Chem. Soc. 1997, 119, 4977-4986) and CPI-TMI (Boger, D. L.; et al. J. Org. Chem. 2000, 65, 4101-4111).

FIG. 5 is a table comparing the various compounds used in a systematic approach to finding the effects of the individual methoxy groups on the indole. Although the CBI-based agents proved to be more sensitive to the removal of the TMI subunit methoxy groups than the DSA- or CPI-based agents, analogous trends were observed. When the cytotoxic potency of CBI-TMI (4, IC₅₀=30 pM) (Warpehoski, M. A.; et al. Chem. Res. Toxicol. 1988, 1, 315-333; Boger D. L.; Yun, W. J. Am. Chem. Soc. 1994, 116, 7996-8006) was compared with the methoxy series 6 (C4 OMe), 7 (C5 OMe), 8 (C6 OMe), 9 (C7 OMe), decreases of 17×, 1.6×, 10×, and 10×, respectively, were observed. Thus, maintenance of the C5 methoxy group with removal of the C6 and C7 methoxy groups with analogue 7 maintained the cytotoxic potency, whereas its removal in 8 and 9 led to a ≧10× reduction in activity.

FIG. 6 is a table of compounds with a wider variety of C5 indole substituents. Addition of a single heavy atom substituent typically resulted in a modest 1.3-40× increase in potency which diminishes as the size of the substituent increased (potency: OH═NH₂>Cl>Br>Me), unsaturated C5 substituents proved more potent than the corresponding saturated counterparts (potency: C≡CH═CH═CH₂>Et) consistent with restrictions on the optimal size of the C5 substiuent, extension of the rigid length of the unsaturated C5 substiuents smoothly follows the trend 0<1<2>3 heavy atoms, and branching at the site of C5 attachment with a small hydrophobic group may increase potency (MeC═CH₂>HC═CH₂). In each case, a C5 substituent was more potent than the corresponding C7 substituent, and the impact of the C5 substituent on potency appears to be related simply to its presence and shape characteristics while being relatively independent of its electronic properties (e.g., OMe=CN).

FIG. 7 is a table showing a series of amine and amide derivatives of 5-aminoindole. Notably the amide derivatives 44-47 proved to be potent cytotoxic agents (IC₅₀=30 pM) being slightly more potent than 7 (C5 OMe, IC₅₀=50 pM) and equivalent with (+)-CBI-TMI (IC₅₀=30 pM) even though they contain a single C5 substituent. This activity was invariant within the range of the amides examined (44=45=46), and was unaffected by N-methylation (44=47) indicating H-bonding plays no role in their activity. Notably, this series did not exhibit the trends detailed by Lown for the CPI-pyrrole conjugates (NHCOPr>NHCOEt>NHCOMe).

FIG. 8 is a table showing a series of three nitro-substituted indole derivatives and their relative potencies. The smooth trend of 49>50>51 was observed. The distinction being that each was more potent than the corresponding methoxy derivative with 49 (C5 NO₂, IC₅₀=20 pM) not only surpassing the potency of 7 (C5 OMe, IC₅₀=50 pM) but also surpassing the potency of even (+)-CBI-TMI (4, IC₅₀=30 pM). Nonetheless, the relatively small distinction between the strongly electron-withdrawing NO₂ versus electron-donating OMe substituent (C5=2.5×, C6=5×, C7=1.5×) suggest electronic effects (Boger, D. L.; Yun, W. J. Am. Chem. Soc. 1994, 116, 5523-5524) modulated through the indole ring may attenuate activity but only to minor extent.

FIG. 9 is a table with carbonyl-containing substituents on the indole ring. Most significant of the observations was the behavior of the C5 acyl derivatives 54 and 56. A 1000-fold increase in potency over (+)-CBI-indole (5) was observed with 54 and 56 representing an additional ≧10-fold increase in potency beyond most C5 substituted derivatives described above. Analogues 54 and 56 are exceptionally potent cytotoxic compounds (IC₅₀=2-3 pM) exceeding the activity of CBI-TMI (4, 30 pM), CC-1065 (1, 20 pM), and duocarmycin SA (3, 6-10 pM). Interestingly, the analogue 57 containing a propyl chain reverted to the potency characteristic of the C5 substituted analogues (IC₅₀=20 pM), and did not show the further enhancement observed with 54 and 56.

FIG. 10 is a drawing showing the location of the antibiotic in the minor groove after alkylation.

FIG. 11 is a table with two sulfone substituted indole derivatives. Like 54, the C5 methanesulfonyl derivative 64 proved exceptionally potent (IC₅₀=3 pM) being 15-20 fold more active than the corresponding thiomethyl derivative 21 (IC₅₀=50 pM) or methoxy derivative 7 (IC₅₀=50 pM) and exceeding the activity of even 1-5. Unlike 56, the corresponding ethanesulfonyl derivative, while being a potent cytotoxic agent (IC₅₀=40 pM), did not exhibit this exceptional activity, and was only slightly more active than the corresponding thioethyl derivative 23 (IC₅₀=100 pM).

FIG. 12 is a table comparing the potencies of the tricyclic indole derivatives. The results of an examination of such alternative tricyclic systems that represent rigid or conformationally restricted analogues of the potent derivatives are summarized in FIG. 12. The cyclic structures representing substitutions at the 4,5 positions were expected to be superior to the 5,6 analogues due to their ability to adopt a conformation embodying the embedded minor groove substituent. Where examined, the 4,5-isomer was more potent than the corresponding 5,6-isomer (72 vs. 73 and 74 vs. 75), and introduction of unsaturation resulted in rather dramatic losses in activity (>10-fold, 66 vs. 69 and 71 vs. 70). Most importantly, the 4,5 constrained analogues (but not the 5,6 constrained analogues) typically matched or exceeded the potency of the corresponding unconstrained analogue.

FIG. 13 is a table showing the potency of the analogs which have a linear or angularly fused benzene ring on the indole. The linear extension with (+)-78 resulted in a modest 5-fold increase in potency whereas the angular extension with (+)-77 resulted in a modest 2-fold reduction when compared to (+)-CBI-indole (R═H) and a more significant 6-8 fold reduction relative to the fused pyrrole 69 or the fused furan 71. As expected, the corresponding unnatural enantiomers (−)-77 and (−)-78 were found to be approximately 100-1000× less potent.

FIG. 14 is a table with the relative alkylation efficiency and the relative rate of alkylation of selected derivatives. The CBI analogues displayed significant distinctions in both their efficiencies in DNA alkylation and their rates of DNA alkylation that proved to correlate with the relative and absolute trends observed in their cytotoxic potency. Thus, in the case of the simplified and exceptionally potent derivatives 54 and 64, their enhanced cytotoxic potency correlates with an enhanced rate and efficiency of DNA alkylation.

FIG. 15 is a gel that shows the w794 DNA alkylation after 24 h and at 23° C. with four of the analogs at the 3′-ACTGATTAA-5′.

FIG. 16 is a synthetic scheme for the synthesis of indole carboxylic acids 104, 105 and 108. (a) N₃CH₂CO₂Me, NaOMe, MeOH, 1 h, −20° C. to 25° C. then 2 h at 25° C. or 1 h at −5° C. and 2 h from −5° C. to 25° C. (b) p-xylene, 15 min, 140° C. or 3 h, 135° C. (c) LiOH, dioxane, 12 or 18 h, 25° C.

FIG. 17 is a scheme for the synthesis of indole carboxylic acids 112, 113, and 114. (a) Boc₂O, DMAP, CH₂Cl₂, 1 h, 25° C. (b) HSiEt₃, Pd(OAc)₂, CH₂Cl₂, 15 h, 25° C., then TBAF, 2 min, 25° C. (c) NaH, DMF, 10-15 min, 25° C., RBr, 18 h, 25° C. (d) KOH, EtOH, 30 min, 80° C.

FIG. 18 is a scheme for the 5- and 7-trifluoromethoxyindole-2-carboxylic acids (118, 119) were obtained by LiOH hydrolysis of the methyl esters (116, 117), obtained by the Hemetsberger reaction with 3-trifluoromethoxy-benzaldehyde (FIG. 18) (Murakami, Y.; et al. Bull. Chem. Soc. Jpn. 1995, 43, 1281-1286). (a) N₃CH₂CO₂Me, NaOMe, MeOH 1 h, −20° C. to 25° C. then 2 h at 25° C. (b) p-xylene, 30 min, 135° C. (c) LiOH, dioxane, 12 to 15 h, 25° C.

FIG. 19 is a scheme for the synthesis of thio and sulfonyl-substituted indole 2-carboxylic acids 25a, 25b, 26a, 26b and 27. (a) BuLi, THF, 10 min, −78° C., MeSSMe or EtSSEt, 1 h, −78° C., 1 h −78° C. to 0° C. (b) HCl, THF, 2 h, 25° C. (c) N₃CH₂CO₂Me, NaOMe, MeOH 1 h, −5° C. then 2 h, −5° C. to 25° C. (d)ₚ-xylene, 3 h, 130° C. (e) LiOH, dioxane, 12-18 h, 25° C. (f) m-CPBA, CH₂Cl₂, 3 h, −78° C. to 25° C.

FIG. 20 is a scheme for the synthesis of methyl methoxy-substituted indole 2-carboxylic acids 132 and 133. (a) NaBH₄, MeOH, 15 min, 0° C. (b) NaH, THF, 30 min, 0° C. then MeI, 14 h, 0° C. to 25° C. (c) HCl, THF, 30 min, 25° C.

(d) N₃CH₂CO₂Me, NaOMe, MeOH, 1 h, −20° C. to 25° C. then 2 h at 25° C.

(e) p-xylene, 3 h, 135° C. (f) LiOH, dioxane, 9 h, 25° C.

FIG. 21 is a scheme for the synthesis of 5-azidoindole-2-carboxylic acid 136. (a) H₂, Pd/C, EtOAc, 14 h, 25° C. (b) NaNO₂, HCl(aq), 30 min, 0° C. then NaN₃, NaOAc, H₂O, 1 h, 0° C. (c) LiOH(aq), dioxane, 48 h, 25° C.

FIG. 22 is a scheme for the synthesis of 5-cyanoindole-2-carboxylic acid 140. (a) H₂SO₄, EtOH, 18 h, reflux. (b) CuCN, DMF, 7 h, 155° C. (c) KOH, EtOH, 0.75 h, 80° C.

FIG. 23 is a scheme for the synthesis of 7-cyanoindole-2-carboxylic acid 144. (a) Ethyl pyruvate, EtOH, 1 h, 80° C. (b) PPA, 10 min, 150° C. (c) CuCN, DMF, 5 h, 155° C. (d) KOH, EtOH, 1 h, 80° C.

FIG. 24 is a scheme for the synthesis of 2-carboxylic acid indoles 47, 50 and 51. (a) CH₂N₂, THF/Et₂O, 25° C. (b) Ph₃PCH₃′, KHMDS, THF, 8 h, 0° C. to 25° C. (c) LiOH, dioxane, 12 h, 25° C. (d) HCl, THF, 30 min, 25° C. (e) H₂, Pd/C, THF, 1 h, 25° C. (f) KOH, EtOH, 30 min, 80° C.

FIG. 25 is a scheme for the synthesis of 5- and 7-isopropenylindole-2-carboxylic acids 153 and 155. (a) Ph₃PCH₃I, KHMDS, THF, 8 h, 0° C. to 25° C. (b) LiOH, dioxane, 15 h, 24 h, 25° C.

FIG. 26 is a scheme for the synthesis of 5-Ethynylindole-2-carboxylic acid 157. (a) TMS-acetylene, Pd(PPh₃)₄—CuI, Et₃N/CH₃CN, 5 h, 80° C. (b) NaOH, EtOH, 20 min, 25° C. (c) Boc₂O, DMAP, CH₂Cl₂, 1 h, 25° C. (d) LiOH, dioxane, 48 h, 25° C.

FIG. 27 is a scheme for the synthesis of 5-(1-Propynyl)-indole-2-carboxylic acid 159. (a) CH₃CCH, Pd(PhCN)₂Cl₂, CuI, ^{t}Bu₃P, ⁱPr₂NH, dioxane, 48 h, 25° C. (b) LiOH, dioxane, 36 h, 25° C.

FIG. 28 is a scheme for the synthesis of 4-Phenylindole-2-carboxylic acid 63. (a) MnO₂, CH₂Cl₂, 36 h, 25° C. (b) N₃CH₂CO₂Me, NaOMe, MeOH, 1 h, −5° C., then 3 h, 25° C. (c) p-xylene, 4 h, 140° C. (d) KOH, EtOH, 30 min, 80° C.

FIG. 29 is a scheme for the synthesis of 5-Dimethylaminoindole-2-carboxylic acid 165. (a) H₂ (60 psi), 10% Pd/C, 37% formaldehyde, THF, 16 h, 23° C. (b) LiOH, THF, H₂O, MeOH, 2.5 h, 23° C. (c) H₂ (60 psi), 10% Pd/C, CH₃CHO, THF, 16 h, 23° C.

FIG. 30 is a scheme for the synthesis of 5-acetylaminoindole-2-carboxylic acid 172, 5-propionylaminoindole-2-carboxylic acid 173, and 5-butyrylaminoindole-2-carboxylic acid 174. (a) (RCO)₂O, pyridine, 12 h, 0 to 25° C. (b) Cs₂CO₃, EtOH, 2.5 h, 80° C.

FIG. 31 is a scheme for the synthesis of 5-(N-acetyl-N-methylamino) indole-2-carboxylic acid 177 and 5-(N-acetyl-N-ethylamino)indole-2-carboxylic acid 179. (a) Boc₂O, DMAP, CH₂Cl₂, 1 h, 25° C. (b) NaH, DMF, 15 min, 0° C. then MeI, 15 h, 25° C. (c) LiOH, dioxane, 36 h, 25° C. (d) 16% HCl, NaNO₂, 23° C. (e) EtNH₂, H₂O, 23° C. (f) Ac₂O, py, 23° C. (g) Cs₂CO₃, EtOH.

FIG. 32 is a scheme for the synthesis of 5-formylindole-2-carboxylic acid 181 and 7-formylindole-2-carboxylic acid 180. (a) BuLi, THF, 1 h, −78° C. then N-formylpiperidine, 15 min, −78° C. and 6 h from −78° C. to 25° C. (b) N₃CH₂CO₂Me, NaOMe, MeOH, 2 h, −5° C. then 2 h from −5° C. to 25° C. (c) p-xylene, 3 h, 135° C. (d) LiOH, dioxane, 6 to 24 h, 25° C. then HCl, 10 min, 25° C.

FIG. 33 is a scheme for the synthesis of 5-acetylindole-2-carboxylic acid 183, 7-acetylindole-2-carboxylic acid 184, 5-propionylindole-2-carboxylic acid 187, and 5-butyrylindole-2-carboxylic acid 188. (a) RCOCl, AlCl₃, MeNO₂, 1 h, 25° C. (b) KOH, EtOH, 1 h, 80° C.

FIG. 34 is a scheme for the synthesis of 7-methoxycarbonylindole-2-carboxylic acid 190 and 5-methoxycarbonylindole-2-carboxylic acid 192. The syntheses of 5-ethoxycarbonylindole-2-carboxylic acid 193, 5-methylcarbamoyl indole-2-carboxylic acid 194, and 5-dimethylcarbamoylindole-2-carboxylic acid 195. (a) BnOH, EDCl, DMAP, CH₂Cl₂, 1 h, 0° C. and 1 h at 25° C. (b) PDC, DMF, 4 to 5 d, 25° C. (c) CH₂N₂, THF/Et₂O, 5 min, 25° C. (d) H₂, Pd/C, THF, 1 h, 25° C. (e) C₂O₂Cl₂, DMF, CH₂Cl₂, 40 min, 0° C. to 25° C. (f) EtOH, NH₂Me or NHMe₂, CH₂Cl₂, 1 h, 0° C. to 25° C. or 10 min, 0° C.

FIG. 35 is a scheme for the synthesis of 5-carbamoylindole-2-carboxylic acid 196. (a) H₂O₂, K₂CO₃, DMSO, 1 h, 0° C. (b) Cs₂CO₃, EtOH, 2.5 h, 80° C.

FIG. 36 is a scheme for the synthesis of 1,2-Dihydropyrrolo[3,2-e]benzofuran-7-carboxylic acid (99). (a) MnO₂, CH₂Cl₂, 36 h, 25° C. (b) N₃CH₂CO₂Me, NaOMe, MeOH, 1 h, −5° C., then 3 h, 25° C.; p-xylene, 4 h, 140° C. (c) KOH, EtOH, 30 min, 80° C.

FIG. 37 is a scheme for the synthesis of pyrrolo[3,2-e]benzofuran-7-carboxylic acid 102. (a) N₃CH₂CO₂Me, NaOMe, MeOH, 1 h, −5° C., then 3 h, 25° C. (b)ₚ-xylene, 4 h, 140° C. (c) LiOH, EtOH, THF, H₂O.

FIG. 38 is a scheme for the synthesis of 6-oxo-cyclopenten[e]indole-2-carboxylic acid 107. (a) ^{t}BuOK, DMF, 10 min, 0° C. then Me₂SO₄, 10 min, 0° C. (b) 03, CH₂Cl₂/pyridine, 30 min at −78° C. then Me₂S, 15 min, −78° C. to 0° C. (c) N₃CH₂CO₂Me, NaOMe, MeOH, 2 h, 0° C. to 25° C. (d) p-xylene, 3 h, 130° C. (e) NaOH, MeOH/THF, 24 h, 25° C. (f) C₂Cl₂O₂, DMF, CH₂Cl₂, 1 h, 0° C. to 25° C. (g) AlCl₃, CH₂ClCH₂Cl, 2 h, 0° C. to 25° C. (h) LiOH, dioxane, 14 h, 25° C.

FIG. 39 is a scheme for the synthesis of 6-oxo-cyclopenten[f]indole-2-carboxylic acid 112. (a) CH₂N₂, THF/Et₂O, 10 min, 25° C. (b) H₂/PtO₂, EtOAc, 40 min, 25° C. (c) N₃CH₂CO₂Me, NaOMe, MeOH, 2 h, 0° C. to 25° C. (d) p-xylene, 3 h, 130° C. (e) NaOH, MeOH/THF, 15 h, 25° C. (f) C₂Cl₂O₂, DMF, CH₂Cl₂, 1 h, 0° C. to 25° C. (g) AlCl₃, CH₂ClCH₂Cl, 2 h, 0° C. to 25° C. (h) LiOH, dioxane, 12 h, 25° C.

FIG. 40 is a scheme for the synthesis of [1,3]dioxolo[e]indole-2-carboxylic acid 115. (a) N₃CH₂CO₂Me, NaOMe, MeOH, 1 h, −5° C., then 3 h, 25° C. (b) p-xylene, 14 h, 130° C. (c) LiOH, THF, MeOH, H₂O, 2 h, 80° C.

FIG. 41 is a scheme for the synthesis of [1,3]Dioxolo[f]indole-2-carboxylic acid 118. (a) N₃CH₂CO₂Me, NaOMe, MeOH 1 h from −20° C. to 25° C. then 2 h at 25° C. (b) p-xylene, 1 h, 135° C. (c) LiOH, dioxane, 15 h, 25° C.

FIG. 42 is a scheme for the synthesis of cyclopenten[e]indole-2-carboxylic acid 223. (a) TiCl₄, α,α-dichloromethyl methyl ether, 0.5 h, 0-25° C.; prep HPLC. (b) N₃CH₂CO₂Me, NaOMe, MeOH, 1 h, −5° C., then 3 h, 25° C. (c) p-xylene, 4 h, 140° C. (d) LiOH, THF, MeOH, H₂O, 2 h, 80° C.

FIG. 43 is a scheme for the synthesis of benz[e]indole-2-carboxylic acid 226. (a) NaOMe in MeOH, 10 min, −25° C.; 2 h, 0° C. (b) p-xylene, 4 h, reflux. (c) KOH in EtOH, 0.5 h, 80° C.

FIG. 44 is a scheme for the synthesis of benz[f]indole-2-carboxylic acid 232. (a) NaOEt, EtOH, 12 h, reflux. (b) Phthalic anhydride, AlCl₃, DCE, 0.5 h, reflux. (c) HSiEt₃, TFA, 24 h, 25° C. (d) NaBH₄, BF₃.Et₂O, THF, 4.5 h, 25° C. (e) MnO₂, CH₂Cl₂, 2 h, reflux. (f) TFA, CH₂Cl₂, 40 min, 0° C. (g) KOH, EtOH, 0.5 h, 80° C.

FIG. 45 is a scheme showing the procedure for attaching the alkylation unit to the indol-2-carboxylic acids and for completing the spirocyclization to give the completed CBI alkylation subunit. (a) 4 N HCl/EtOAc, 2 h, 23° C. (b) EDCl, DMF, 14 h, 25° C. (c) DBU, CH₃CN or CH₃CN/THF.

1. A compound having a formula selected from the group consisting of the following structures: 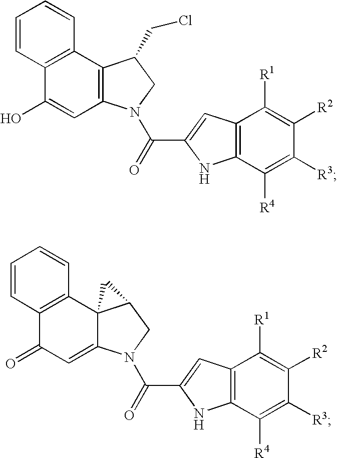
wherein:
R¹ is —H or forms a five membered ring with R²;
R² is selected from the group consisting of —COOR^{a}, —CONR^{b}R^{c}, S(O)NR^{d}, —OCF₃, —NO₂, —CHO, —CH═CH₂, —C(CH₃)═CH₂, and —CH₂OCH₃ or forms a five membered ring with either R¹ or R³;
R³ is selected from the group consisting of —H, —OCF₃, —CHO, —NO₂, —CH═CH₂, —C(CH₃)═CH₂, and —CH₂OCH₃, or forms a five membered ring with R²; and
R⁴ is selected from the group consisting of —H, —OCF₃, —CHO, —NO₂, —CH═CH₂, —C(CH₃)═CH₂, and —CH₂OCH₃;
wherein:
R^{a}, R^{b}, R^{c}, and R^{d} are independently —C₍₁₋₆₎ alkyl radicals;
n is 1 or 2;
the five membered ring being of a type selected from the group consisting of five membered rings having 5 ring carbons having a ketone substitution, five membered rings having 4 ring carbons and 1 ring oxygen, and five membered rings having 3 ring carbons and 2 nonadjacent ring oxygens, each of the five membered rings having 1 ring unsaturation;
with the following provisos:
at least one of R¹, R², R³, and R⁴ is not —H; and
if R² forms a five membered ring, then R² forms a five membered ring with only one of R¹ or R³.

2. A compound according to claim 1 having a formula selected from the group consisting of the following structures: 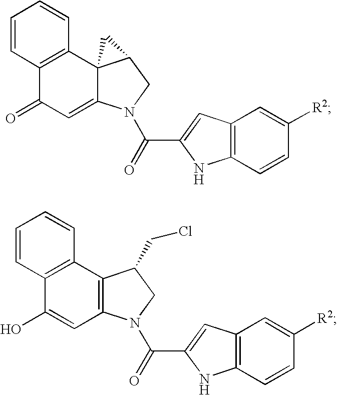
wherein R² is —COOR^{a}, wherein R^{a} is a —C₍₁₋₆₎ alkyl radical.

3. A compound according to claim 2 represented by the following structure:

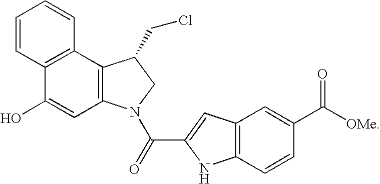

4. A compound according to claim 2 represented by the following structure:

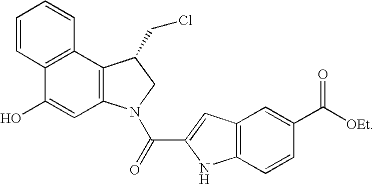

5. A compound according to claim 1 having a formula selected from the group consisting of the following structures: 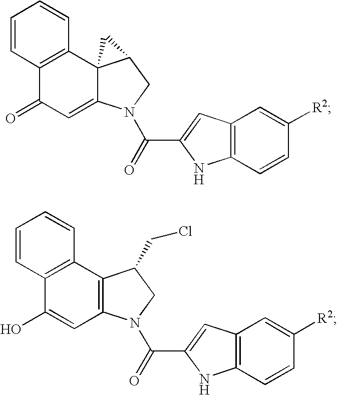
wherein R² is —CONR^{b}R^{c}; and
wherein R^{b} and R^{c} are independently —(C₁-C₆)alkyl radicals.

6. A compound according to claim 1 having the following structure:

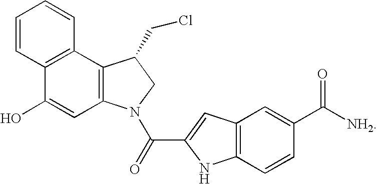

7. A compound according to claim 1 having the following structure:

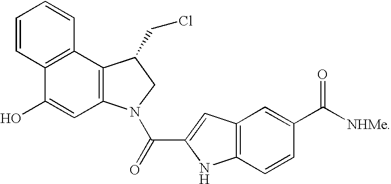

8. A compound according to claim 5 represented by the following structure:

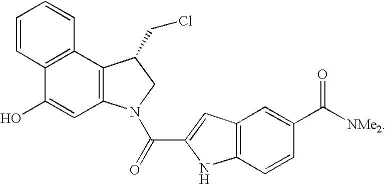

9. A compound according to claim 1 having a formula selected from the group consisting of the following structures: 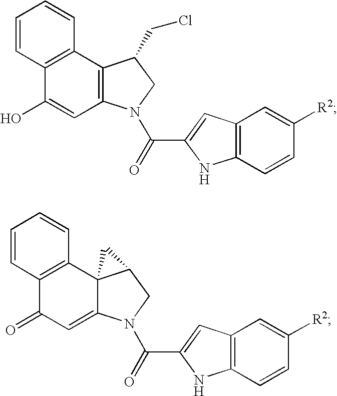
wherein R² is —S(O)ₙR^{d}; and
wherein n is 1 or 2; and R^{d} is a —(C₁-C₆) radical.

10. A compound according to claim 9 represented by the following structure:

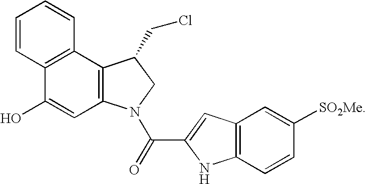

11. A compound according to claim 9 represented by the following structure:

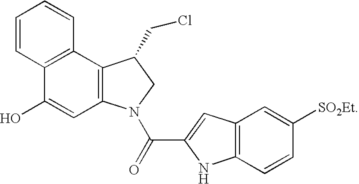

12. A compound according to claim 1 having a formula selected from the group consisting of the following structures: 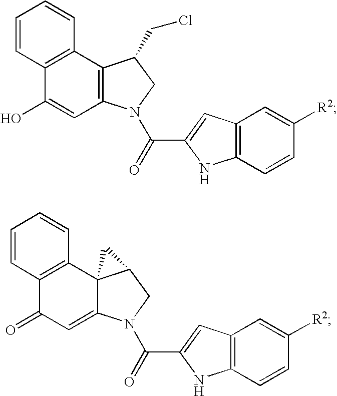
wherein R² is —SR^{e}; and
wherein R^{e} is a —(C₁-C₆)alkyl radical.

13. A compound according to claim 12 represented by the following structure:

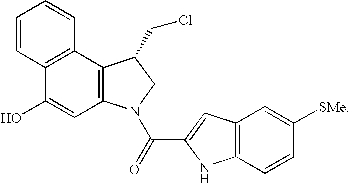

14. A compound according to claim 12 represented by the following structure:

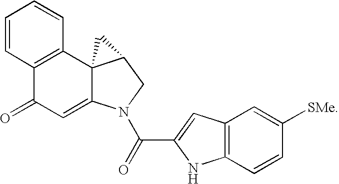

15. A compound according to claim 12 represented by the following structure:

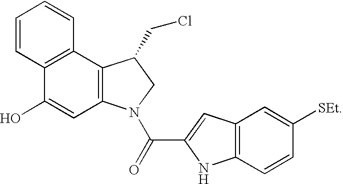

16. A compound according to claim 12 represented by the following structure:

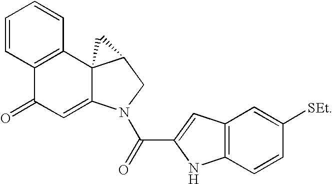

17. A compound according to claim 1 having a formula selected from the group consisting of the following structures: 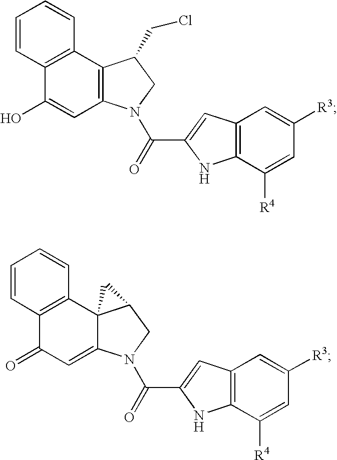
wherein R² is selected from the group consisting of —OCF₃ and —H; and R⁴ is selected from the group consisting of —OCF₃ and H;
with the following provisos:
if R² is —OCF₃, then R⁴ is —H; and
if R⁴ is —OCF₃, then R² is —H.

18. A compound according to claim 17 represented by the following structure:

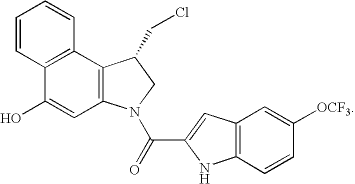

19. A compound according to claim 17 represented by the following structure:

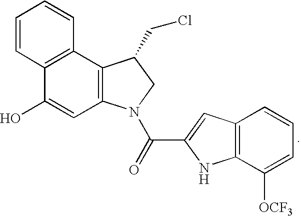

20. A compound according to claim 1 having a formula selected from the group consisting of the following structures: 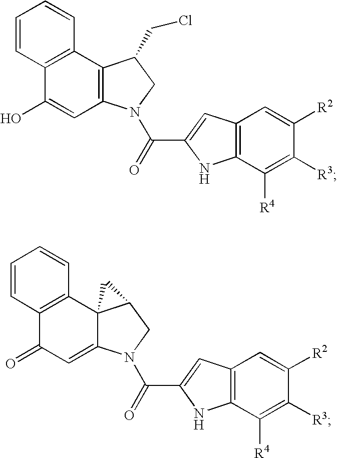
wherein:
R² is selected from the group consisting of —NO₂ and —H;
R³ is selected from the group consisting of —NO₂ and —H;
R⁴ is selected from the group consisting of —NO₂ and —H;
with the following provisos:
if R² is —NO₂, then R³ and R⁴ are —H;
if R³ is —NO₂, then R² and R⁴ are —H; and
if R⁴ is —NO₂, then R² and R³ are —H.

21. A compound according to claim 20 represented by the following structure:

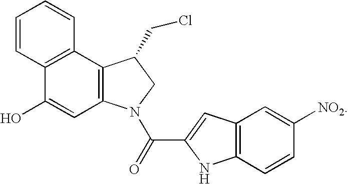

22. A compound according to claim 20 represented by the following structure:

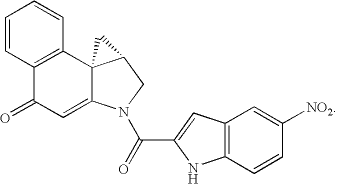

23. A compound according to claim 20 represented by the following structure:

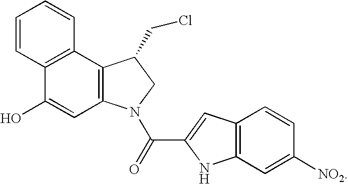

24. A compound according to claim 20 represented by the following structure:

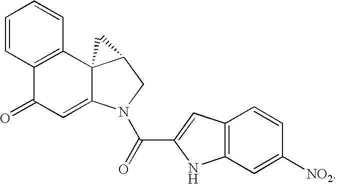

25. A compound according to claim 20 represented by the following structure:

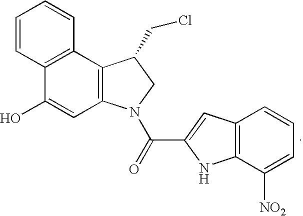

26. A compound according to claim 20 represented by the following structure:

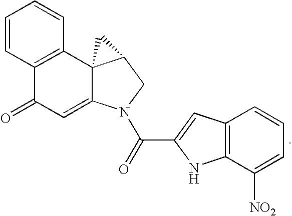

27. A compound according to claim 1 having a formula selected from the group consisting of the following structures: 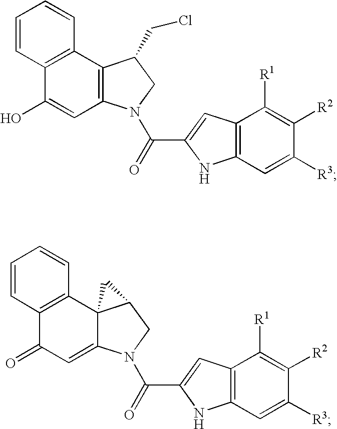
wherein:
R¹ is —H or forms a five membered ring with R²;
R³ is —H or forms a five membered ring with R²; and
R² forms a five membered ring with either R¹ or R³;
the five membered ring is of a type selected from the group consisting of five membered rings having 5 ring carbons having a ketone substitution, five membered rings having 4 ring carbons and 1 ring oxygen, and five membered rings having 3 ring carbons and 2 nonadjacent ring oxygens, each of the five membered rings having 1 ring unsaturation
with the following proviso:
R² forms a five membered ring with only one of R¹ or R³.

28. A compound according to claim 27 represented by the following structure:

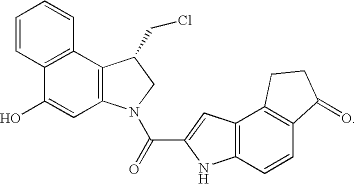

29. A compound according to claim 27 represented by the following structure:

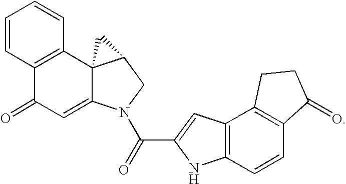

30. A compound according to claim 27 represented by the following structure:

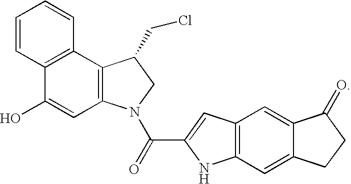

31. A compound according to claim 27 represented by the following structure:

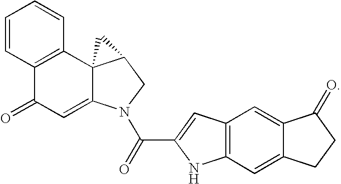

32. A compound according to claim 27 represented by the following structure:

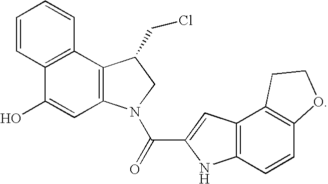

33. A compound according to claim 27 represented by the following structure:

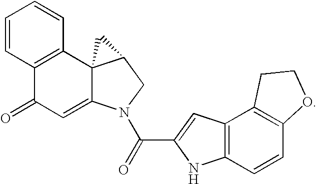

34. A compound according to claim 27 represented by the following structure:

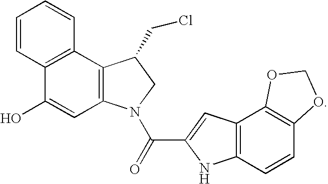

35. A compound according to claim 27 represented by the following structure:

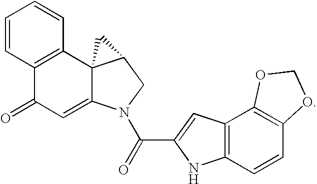

36. A compound according to claim 1 having a formula selected from the group consisting of the following structures 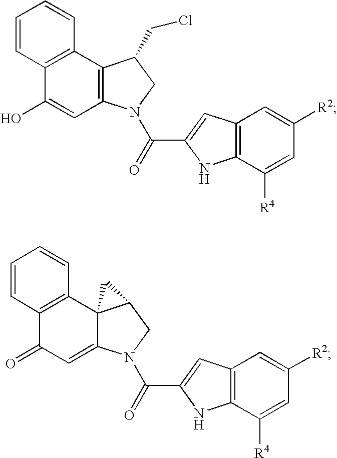
wherein:
R² is selected from the group consisting of —CHO and —H; and
R⁴ is selected from the group consisting of —CHO and —H;
with the following provisos:
if R² is —CHO, then R⁴ is —H; and
if R² is —H, then R⁴ is —CHO.

37. A compound according to claim 36 represented by the following structure:

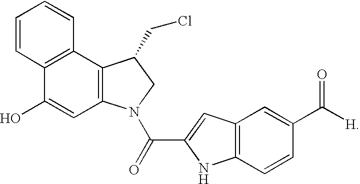

38. A compound according to claim 36 represented by the following structure:

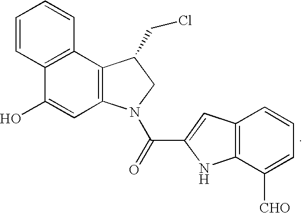

39. A compound according to claim 36 represented by the following structure:

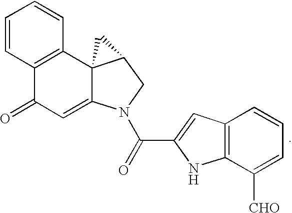

40. A compound according to claim 1 having a formula selected from the group consisting of the following structures: 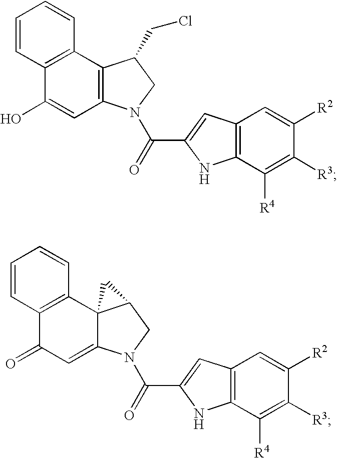
wherein:
R² is selected from the group consisting of —H, —CH═CH₂—C(CH₃)═CH₂, and —CH₂OCH₃;
R³ is selected from the group consisting of —H; —CH═CH₂, —C(CH₃)═CH₂, and —CH₂OCH₃; and
R⁴ is selected from the group consisting of —H; —CH═CH₂, —C(CH₃)═CH₂, and —CH₂OCH₃;
with the following proviso:
at least one of R², R³, and R⁴ is not —H.

41. A compound according to claim 40 represented by the following structure:

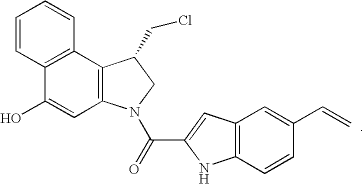

42. A compound according to claim 40 having the following structure:

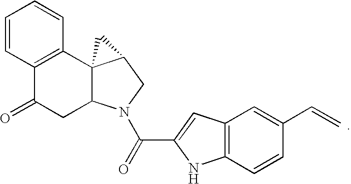

43. A compound according to claim 40 represented by the following structure:

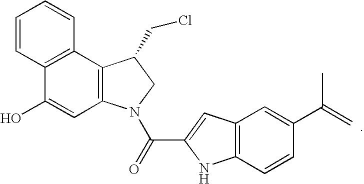

44. A compound according to claim 40 represented by the following structure:

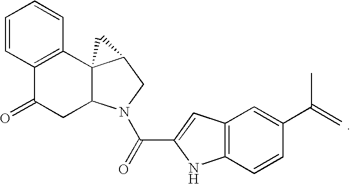

45. A compound according to claim 40 represented by the following structure:

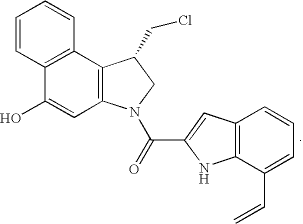

46. A compound according to claim 40 represented by the following structure:

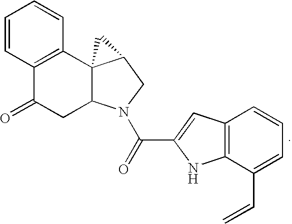

47. A compound according to claim 40 represented by the following structure:

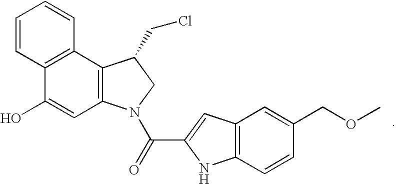

48. A compound according to claim 40 represented by the following structure: [chemical structure]


SEQUENCE LISTING (2 sequences)

SEQ ID NO: 1 - DNA, 11 bp, Artificial Sequence
  This artificial 11-mer is synthesized to form a specific AT-rich region for site-specific DNA duplex N-alkylation at the N3 position of adenine.
gactaattgac

SEQ ID NO: 2 - DNA, 11 bp, Artificial Sequence
  This artificial 11-mer is synthesized to form a specific AT-rich region for site-specific DNA duplex N-alkylation at the N3 position of adenine. Alkylation occurs on this strand at the 3rd adenine residue.
gtcaattagtc